SEQUENCE LISTING (91 sequences)

SEQ ID NO: 1 - protein, 111 aa, synthetic construct
  REGION 1..111: note = synthetic polypeptide
DIVLTQSPASLAVSLGQRATISCRASKSVSTSSYSYMHWYQQKPGQPPKLLIKYASYLESGVPARFSGSGSGTDFTLNIHPVEEEDAATYYCQHSREFPWTFGGGTKLEIK

SEQ ID NO: 2 - protein, 111 aa, synthetic construct
  REGION 1..111: note = synthetic polypeptide
DIVLTQSPASLAVSLGQRATISCRASKSVSTSSYSYMHWYQQKPGQPPKLLIKYASYLESGVPARFSGSGSGTDFHLNIHPVEEEDAATYYCQHSREFPWTFGGGTKLELK

SEQ ID NO: 3 - protein, 111 aa, synthetic construct
  REGION 1..111: note = synthetic polypeptide
DIQMTQSPSSLSASVGDRVTITCRASKSVSTSSYSYLAWYQQKPEKAPKLLIKYASYLQSGVPSRFSGSGSGTDFTLTISSLQPEDFATYYCQHSREFPWTFGAGTKLELK

SEQ ID NO: 4 - protein, 111 aa, synthetic construct
  REGION 1..111: note = synthetic polypeptide
DIQMTQSPSSLSASVGDRVTISCRASKSVSTSSYSYMHWYQQKPEKAPKLLIKYASYLQSGVPSRFSGSGSGTDFTLTISSLQPEDVATYYCQHSREFPWTFGAGTKLELK

SEQ ID NO: 5 - protein, 111 aa, synthetic construct
  REGION 1..111: note = synthetic polypeptide
DIVLTQSPASLAVSPGQRATITCRASKSVSTSSYSYMHWYQQKPGQPPKLLIYYASYLESGVPARFSGSGSGTDFTLTINPVEANDTANYYCQHSREFPWTFGQGTKVEIK

SEQ ID NO: 6 - protein, 116 aa, synthetic construct
  REGION 1..116: note = synthetic polypeptide
EVQLVESGGGLVKPGGSRKLSCAASGFTFSDYGMHWVRQAPEKGLEWVAYISSGSSTIYYADTVKGRFTISRDNAKNTLFLQMTSLRSEDTAMYYCARRGLLLDYWGQGTTLTVSS

SEQ ID NO: 7 - protein, 116 aa, synthetic construct
  REGION 1..116: note = synthetic polypeptide
EVQLVESGGGLVKPGGSRKLSCAASGFTFSNYGMHWVRQAPEKGLEWVAYISSGSSTIYYADTVKGRFTISRDNAKNTLFLQMTSLRSEDTAMYYCARRGLLLDYWGQGTTLTVSS

SEQ ID NO: 8 - protein, 5 aa, synthetic construct
  REGION 1..5: note = synthetic polypeptide
DYGMH

SEQ ID NO: 9 - protein, 116 aa, synthetic construct
  REGION 1..116: note = synthetic polypeptide
EVQLVQSGGGLIQPGGSLRLSCAASGFTFSNYGMHWVRQAPGKGLEWVSYISSGSSTIYYADSVKGRFTISRDNSKNTLYLQMNSLRAEDTAVYYCARRGLLLDYWGQGTTVTVSS

SEQ ID NO: 10 - protein, 17 aa, synthetic construct
  REGION 1..17: note = synthetic polypeptide
YISSGSSTIYYADTVKG

SEQ ID NO: 11 - protein, 116 aa, synthetic construct
  REGION 1..116: note = synthetic polypeptide
EVQLVESGGGLIQPGGSLRLSCAASGFTFSNYGMHWVRQAPGKGLEWVSYISSGSSTIYYADSVKGRFTISRDNSKNTLYLQMTSLRAEDTAVYYCARRGLLLDYWGQGTTLTVSS

SEQ ID NO: 12 - protein, 112 aa, synthetic construct
  REGION 1..112: note = synthetic polypeptide
DIVLTQSPASLAAVSLGERATISYRASKSVSTSGYSYMHWNQQKPGQAPRLLIYLVSNLESGVPARFSGSGSGTDFTLNIHPVEEEDAATYYCQHIRELDTFFGGGTKLEIK

SEQ ID NO: 13 - protein, 15 aa, synthetic construct
  REGION 1..15: note = synthetic polypeptide
RASKSVSTSGYSYMH

SEQ ID NO: 14 - protein, 7 aa, synthetic construct
  REGION 1..7: note = synthetic polypeptide
LVSNLES

SEQ ID NO: 15 - protein, 9 aa, synthetic construct
  REGION 1..9: note = synthetic polypeptide
QHIRELDTF

SEQ ID NO: 16 - protein, 120 aa, synthetic construct
  REGION 1..120: note = synthetic polypeptide
QLKLVESGGGLVKPGGSLKLSCAASGFTFSSYTMSWVRQTPAKRLEWVATISSGGGSTYYPDSVKGRFTISRDNARNTLYLQMSSLRSEDTAMYYCARRAYSKRGAMDYWGQGTSVTVSS

SEQ ID NO: 17 - protein, 5 aa, synthetic construct
  REGION 1..5: note = synthetic polypeptide
SYTMS

SEQ ID NO: 18 - protein, 17 aa, synthetic construct
  REGION 1..17: note = synthetic polypeptide
TISSGGGSTYYPDSVKG

SEQ ID NO: 19 - protein, 11 aa, synthetic construct
  REGION 1..11: note = synthetic polypeptide
RAYSKRGAMDY

SEQ ID NO: 20 - protein, 7 aa, synthetic construct
  REGION 1..7: note = synthetic polypeptide
GQSSRSS

SEQ ID NO: 21 - protein, 18 aa, synthetic construct
  REGION 1..18: note = synthetic polypeptide
GQSSRSSSGGGSSGGGGS

SEQ ID NO: 22 - protein, 4 aa, synthetic construct
  REGION 1..4: note = synthetic polypeptide
GSGS

SEQ ID NO: 23 - protein, 4 aa, synthetic construct
  REGION 1..4: note = synthetic polypeptide
GGGS

SEQ ID NO: 24 - protein, 10 aa, synthetic construct
  REGION 1..10: note = synthetic polypeptide
GGGGSGGGGS

SEQ ID NO: 25 - protein, 20 aa, synthetic construct
  REGION 1..20: note = synthetic polypeptide
GGGGSGGGGSGGGGSGGGGS

SEQ ID NO: 26 - protein, 15 aa, synthetic construct
  REGION 1..15: note = synthetic polypeptide
GGGGSGGGGSGGGGS

SEQ ID NO: 27 - protein, 274 aa, synthetic construct
  REGION 1..274: note = synthetic polypeptide
AGIHDIVLTQSPASLAVSLGQRATISCRASKSVSTSSYSYMHWYQQKPGQPPKLLIKYASYLESGVPARFSGSGSGTDFTLNIHPVEEEDAATYYCQHSREFPWTFGGGTKLEIKRADAAPGGGGSGGGGSGGGGSEVQLVESGGGLVKPGGSRKLSCAASGFTFSNYGMHWVRQAPEKGLEWVAYISSGSSTIYYADTVKGRFTISRDNAKNTLFLQMTSLRSEDTAMYYCARRGLLLDYWGQGTTLTVSSLEQKLISEEDLNSAVDHHHHHH

SEQ ID NO: 28 - protein, 541 aa, synthetic construct
  REGION 1..541: note = synthetic polypeptide
AGIHDIVLTQSPASLAVSLGQRATISCRASKSVSTSSYSYMHWYQQKPGQPPKLLIKYASYLESGVPARFSGSGSGTDFTLNIHPVEEEDAATYYCQHSREFPWTFGGGTKLEIKRADAAPGGGGSGGGGSGGGGSEVQLVESGGGLVKPGGSRKLSCAASGFTFSNYGMHWVRQAPEKGLEWVAYISSGSSTIYYADTVKGRFTISRDNAKNTLFLQMTSLRSEDTAMYYCARRGLLLDYWGQGTTLTVSSASTKGPSVFPLAPLESSGSDIVLTQSPASLAVSLGQRATISCRASKSVSTSSYSYMHWYQQKPGQPPKLLIKYASYLESGVPARFSGSGSGTDFTLNIHPVEEEDAATYYCQHSREFPWTFGGGTKLEIKRADAAPGGGGSGGGGSGGGGSEVQLVESGGGLVKPGGSRKLSCAASGFTFSNYGMHWVRQAPEKGLEWVAYISSGSSTIYYADTVKGRFTISRDNAKNTLFLQMTSLRSEDTAMYYCARRGLLLDYWGQGTTLTVSSLEQKLISEEDLNSAVDHHHHHH

SEQ ID NO: 29 - protein, 808 aa, synthetic construct
  REGION 1..808: note = synthetic polypeptide
AGIHDIVLTQSPASLAVSLGQRATISCRASKSVSTSSYSYMHWYQQKPGQPPKLLIKYASYLESGVPARFSGSGSGTDFTLNIHPVEEEDAATYYCQHSREFPWTFGGGTKLEIKRADAAPGGGGSGGGGSGGGGSEVQLVESGGGLVKPGGSRKLSCAASGFTFSNYGMHWVRQAPEKGLEWVAYISSGSSTIYYADTVKGRFTISRDNAKNTLFLQMTSLRSEDTAMYYCARRGLLLDYWGQGTTLTVSSASTKGPSVFPLAPLESSGSDIVLTQSPASLAVSLGQRATISCRASKSVSTSSYSYMHWYQQKPGQPPKLLIKYASYLESGVPARFSGSGSGTDFTLNIHPVEEEDAATYYCQHSREFPWTFGGGTKLEIKRADAAPGGGGSGGGGSGGGGSEVQLVESGGGLVKPGGSRKLSCAASGFTFSNYGMHWVRQAPEKGLEWVAYISSGSSTIYYADTVKGRFTISRDNAKNTLFLQMTSLRSEDTAMYYCARRGLLLDYWGQGTTLTVSSASTKGPSVFPLAPLESSGSDIVLTQSPASLAVSLGQRATISCRASKSVSTSSYSYMHWYQQKPGQPPKLLIKYASYLESGVPARFSGSGSGTDFTLNIHPVEEEDAATYYCQHSREFPWTFGGGTKLEIKRADAAPGGGGSGGGGSGGGGSEVQLVESGGGLVKPGGSRKLSCAASGFTFSNYGMHWVRQAPEKGLEWVAYISSGSSTIYYADTVKGRFTISRDNAKNTLFLQMTSLRSEDTAMYYCARRGLLLDYWGQGTTLTVSSLEQKLISEEDLNSAVDHHHHHH

SEQ ID NO: 30 - protein, 5 aa, synthetic construct
  REGION 1..5: note = synthetic polypeptide
NYGMH

SEQ ID NO: 31 - protein, 17 aa, synthetic construct
  REGION 1..17: note = synthetic polypeptide
YISSSSSTIYYADSVKG

SEQ ID NO: 32 - protein, 17 aa, synthetic construct
  REGION 1..17: note = synthetic polypeptide
YISSGSSTIYYADSVKG

SEQ ID NO: 33 - protein, 7 aa, synthetic construct
  REGION 1..7: note = synthetic polypeptide
RGLLLDY

SEQ ID NO: 34 - protein, 15 aa, synthetic construct
  REGION 1..15: note = synthetic polypeptide
RASKSVSTSSYSYMH

SEQ ID NO: 35 - protein, 15 aa, synthetic construct
  REGION 1..15: note = synthetic polypeptide
RASKTVSTSSYSYMH

SEQ ID NO: 36 - protein, 7 aa, synthetic construct
  REGION 1..7: note = synthetic polypeptide
YASYLES

SEQ ID NO: 37 - protein, 9 aa, synthetic construct
  REGION 1..9: note = synthetic polypeptide
QHSREFPWT

SEQ ID NO: 38 - protein, 8 aa, synthetic construct
  REGION 1..8: note = synthetic polypeptide
GFTFSNYG

SEQ ID NO: 39 - protein, 8 aa, synthetic construct
  REGION 1..8: note = synthetic polypeptide
ISSSSSTI

SEQ ID NO: 40 - protein, 8 aa, synthetic construct
  REGION 1..8: note = synthetic polypeptide
ISSGSSTI

SEQ ID NO: 41 - protein, 9 aa, synthetic construct
  REGION 1..9: note = synthetic polypeptide
ARRGLLLDY

SEQ ID NO: 42 - protein, 10 aa, synthetic construct
  REGION 1..10: note = synthetic polypeptide
KSVSTSSYSY

SEQ ID NO: 43 - protein, 10 aa, synthetic construct
  REGION 1..10: note = synthetic polypeptide
KTVSTSSYSY

SEQ ID NO: 44 - skipped

SEQ ID NO: 45 - protein, 116 aa, synthetic construct
  REGION 1..116: note = synthetic polypeptide
EVQLQESGGGVVQPGGSLRLSCAASGFTFSNYGMHWIRQAPGKGLEWVSYISSGSSTIYYADSVKGRFTISRDNSKNTLYLQMNSLRSEDTAVYYCARRGLLLDYWGQGTLVTVSS

SEQ ID NO: 46 - protein, 116 aa, synthetic construct
  REGION 1..116: note = synthetic polypeptide
EVQLVESGGGLVQPGGSLRLSCAASGFTFSNYGMHWVRQAPGKGLEWVSYISSSSSTIYYADSVKGRFTISRDNAKNSLYLQMNSLRAEDTAVYYCARRGLLLDYWGQGTTVTVSS

SEQ ID NO: 47 - protein, 116 aa, synthetic construct
  REGION 1..116: note = synthetic polypeptide
EVQLVESGGGVVQPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSS

SEQ ID NO: 48 - protein, 116 aa, synthetic construct
  REGION 1..116: note = synthetic polypeptide
EVQLVESGGGDVKPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSS

SEQ ID NO: 49 - protein, 116 aa, synthetic construct
  REGION 1..116: note = synthetic polypeptide
EVQLVESGGGLVQPGGSLRLSCSASGFTFSNYGMHWVRQAPGKGLEYVSYISSGSSTIYYADTVKGRFTISRDNSKNTLYLQMSSLRAEDTAVYYCVKRGLLLDYWGQGTLVTVSS

SEQ ID NO: 50 - protein, 116 aa, synthetic construct
  REGION 1..116: note = synthetic polypeptide
EVQLVESGGGLVQPGGSLRLSCAASGFTFSNYGMHWVRQAPGKGLEWVSYISSGSSTIYYADSVKGRFTISRDNAKNSLYLQMNSLRAEDTAVYYCARRGLLLDYWGQGTTVTVSS

SEQ ID NO: 51 - protein, 116 aa, synthetic construct
  REGION 1..116: note = synthetic polypeptide
EVQLVESGGGVVQPGGSLRLSCAASGFTFSNYGMHWVRQAPEKGLEWVSYISSGSSTIYYADSVKGRFTISRDNSKNTLYLQMNSLRAEDTAVYYCARRGLLLDYWGQGTTVTVSS

SEQ ID NO: 52 - protein, 116 aa, synthetic construct
  REGION 1..116: note = synthetic polypeptide
EVQLVESGGGDVKPGGSLRLSCAASGFTFSNYGMHWVRQAPEKGLEWVSYISSGSSTIYYADSVKGRFTISRDNSKNTLYLQMNSLRAEDTAVYYCARRGLLLDYWGQGTTVTVSS

SEQ ID NO: 53 - protein, 111 aa, synthetic construct
  REGION 1..111: note = synthetic polypeptide
DIQMTQSPSSLSASLGDRATITCRASKSVSTSSYSYMHWYQQKPGQPPKLLIKYASYLESGVPSRFSGSGSGTDFTLTISSLQPEDAATYYCQHSREFPWTFGGGTKVEIK

SEQ ID NO: 54 - protein, 111 aa, synthetic construct
  REGION 1..111: note = synthetic polypeptide
DIQMTQSPSSLSASLGDRATITCRASKSVSTSSYSYMHWYQQKPGQAPKLLIKYASYLESGVPSRFSGSGSGTDFTLTISSLQPEDFATYYCQHSREFPWTFGQGTKVEIK

SEQ ID NO: 55 - protein, 111 aa, synthetic construct
  REGION 1..111: note = synthetic polypeptide
DIQMTQSPSSLSASVGDRVTITCRASKSVSTSSYSYMHWYQQKPGKAPKLLIKYASYLESGVPSRFSGSGSGTDFTLTISSLQPEDFATYYCQHSREFPWTFGQGTKVEIK

SEQ ID NO: 56 - protein, 111 aa, synthetic construct
  REGION 1..111: note = synthetic polypeptide
DIQMTQSPSSLSASLGDRATITCRASKTVSTSSYSYMHWYQQKPGQPPKLLIKYASYLESGVPSRFSGSGSGTDFTLTISSLQPEDAATYYCQHSREFPWTFGGGTKVEIK

SEQ ID NO: 57 - protein, 111 aa, synthetic construct
  REGION 1..111: note = synthetic polypeptide
DIQMTQSPSSLSASVGDRVTITCRASKTVSTSSYSYMHWYQQKPGKAPKLLIKYASYLESGVPSRFSGSGSGTDFTLTISSLQPEDFATYYCQHSREFPWTFGQGTKVEIK

SEQ ID NO: 58 - protein, 111 aa, synthetic construct
  REGION 1..111: note = synthetic polypeptide
DIQMTQSPSSLSASLGDRATITCRASKTVSTSSYSYMHWYQQKPGQAPKLLIKYASYLESGVPSRFSGSGSGTDFTLTISSLQPEDFATYYCQHSREFPWTFGQGTKVEIK

SEQ ID NO: 59 - protein, 21 aa, synthetic construct
  REGION 1..21: note = synthetic polypeptide
RADAAPGGGGSGGGGSGGGGS

SEQ ID NO: 60 - protein, 19 aa, synthetic construct
  REGION 1..19: note = synthetic polypeptide
ASTKGPSVFPLAPLESSGS

SEQ ID NO: 61 - protein, 515 aa, synthetic construct
  REGION 1..515: note = synthetic polypeptide
DIQMTQSPSSLSASLGDRATITCRASKSVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGLVQPGGSLRLSCAASGFTFSNYGMHWVRQAPGKGLEWVSYISSSSSTIYYADSVKGRFTISRDNAKNSLYLQMNSLRAEDTAVYYCARRGLLLDYWGQGTTVTVSSASTKGPSVFPLAPLESSGSDIQMTQSPSSLSASLGDRATITCRASKSVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGLVQPGGSLRLSCAASGFTFSNYGMHWVRQAPGKGLEWVSYISSSSSTIYYADSVKGRFTISRDNAKNSLYLQMNSLRAEDTAVYYCARRGLLLDYWGQGTTVTVSS

SEQ ID NO: 62 - protein, 515 aa, synthetic construct
  REGION 1..515: note = synthetic polypeptide
DIQMTQSPSSLSASLGDRATITCRASKSVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGVVQPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSSASTKGPSVFPLAPLESSGSDIQMTQSPSSLSASLGDRATITCRASKSVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGVVQPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSS

SEQ ID NO: 63 - protein, 515 aa, synthetic construct
  REGION 1..515: note = synthetic polypeptide
DIQMTQSPSSLSASLGDRATITCRASKSVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGDVKPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSSASTKGPSVFPLAPLESSGSDIQMTQSPSSLSASLGDRATITCRASKSVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGDVKPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSS

SEQ ID NO: 64 - protein, 515 aa, synthetic construct
  REGION 1..515: note = synthetic polypeptide
DIQMTQSPSSLSASLGDRATITCRASKSVSTSSYSYMHWYQQKPGQAPKLLIKYASYLESGVPSRFSGSGSGTDFTLTISSLQPEDFATYYCQHSREFPWTFGQGTKVEIKRADAAPGGGGSGGGGSGGGGSEVQLVESGGGLVQPGGSLRLSCAASGFTFSNYGMHWVRQAPGKGLEWVSYISSSSSTIYYADSVKGRFTISRDNAKNSLYLQMNSLRAEDTAVYYCARRGLLLDYWGQGTTVTVSSASTKGPSVFPLAPLESSGSDIQMTQSPSSLSASLGDRATITCRASKSVSTSSYSYMHWYQQKPGQAPKLLIKYASYLESGVPSRFSGSGSGTDFTLTISSLQPEDFATYYCQHSREFPWTFGQGTKVEIKRADAAPGGGGSGGGGSGGGGSEVQLVESGGGLVQPGGSLRLSCAASGFTFSNYGMHWVRQAPGKGLEWVSYISSSSSTIYYADSVKGRFTISRDNAKNSLYLQMNSLRAEDTAVYYCARRGLLLDYWGQGTTVTVSS

SEQ ID NO: 65 - protein, 515 aa, synthetic construct
  REGION 1..515: note = synthetic polypeptide
DIQMTQSPSSLSASLGDRATITCRASKSVSTSSYSYMHWYQQKPGQAPKLLIKYASYLESGVPSRFSGSGSGTDFTLTISSLQPEDFATYYCQHSREFPWTFGQGTKVEIKRADAAPGGGGSGGGGSGGGGSEVQLVESGGGVVQPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSSASTKGPSVFPLAPLESSGSDIQMTQSPSSLSASLGDRATITCRASKSVSTSSYSYMHWYQQKPGQAPKLLIKYASYLESGVPSRFSGSGSGTDFTLTISSLQPEDFATYYCQHSREFPWTFGQGTKVEIKRADAAPGGGGSGGGGSGGGGSEVQLVESGGGVVQPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSS

SEQ ID NO: 66 - protein, 515 aa, synthetic construct
  REGION 1..515: note = synthetic polypeptide
DIQMTQSPSSLSASLGDRATITCRASKSVSTSSYSYMHWYQQKPGQAPKLLIKYASYLESGVPSRFSGSGSGTDFTLTISSLQPEDFATYYCQHSREFPWTFGQGTKVEIKRADAAPGGGGSGGGGSGGGGSEVQLVESGGGDVKPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSSASTKGPSVFPLAPLESSGSDIQMTQSPSSLSASLGDRATITCRASKSVSTSSYSYMHWYQQKPGQAPKLLIKYASYLESGVPSRFSGSGSGTDFTLTISSLQPEDFATYYCQHSREFPWTFGQGTKVEIKRADAAPGGGGSGGGGSGGGGSEVQLVESGGGDVKPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSS

SEQ ID NO: 67 - protein, 515 aa, synthetic construct
  REGION 1..515: note = synthetic polypeptide
DIQMTQSPSSLSASVGDRVTITCRASKSVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGLVQPGGSLRLSCAASGFTFSNYGMHWVRQAPGKGLEWVSYISSSSSTIYYADSVKGRFTISRDNAKNSLYLQMNSLRAEDTAVYYCARRGLLLDYWGQGTTVTVSSASTKGPSVFPLAPLESSGSDIQMTQSPSSLSASVGDRVTITCRASKSVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGLVQPGGSLRLSCAASGFTFSNYGMHWVRQAPGKGLEWVSYISSSSSTIYYADSVKGRFTISRDNAKNSLYLQMNSLRAEDTAVYYCARRGLLLDYWGQGTTVTVSS

SEQ ID NO: 68 - protein, 515 aa, synthetic construct
  REGION 1..515: note = synthetic polypeptide
DIQMTQSPSSLSASVGDRVTITCRASKSVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGVVQPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSSASTKGPSVFPLAPLESSGSDIQMTQSPSSLSASVGDRVTITCRASKSVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGVVQPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSS

SEQ ID NO: 69 - protein, 515 aa, synthetic construct
  REGION 1..515: note = synthetic polypeptide
DIQMTQSPSSLSASVGDRVTITCRASKSVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGDVKPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSSASTKGPSVFPLAPLESSGSDIQMTQSPSSLSASVGDRVTITCRASKSVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGDVKPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSS

SEQ ID NO: 70 - protein, 515 aa, synthetic construct
  REGION 1..515: note = synthetic polypeptide
DIQMTQSPSSLSASLGDRATITCRASKTVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGLVQPGGSLRLSCAASGFTFSNYGMHWVRQAPGKGLEWVSYISSGSSTIYYADSVKGRFTISRDNAKNSLYLQMNSLRAEDTAVYYCARRGLLLDYWGQGTTVTVSSASTKGPSVFPLAPLESSGSDIQMTQSPSSLSASLGDRATITCRASKTVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGLVQPGGSLRLSCAASGFTFSNYGMHWVRQAPGKGLEWVSYISSGSSTIYYADSVKGRFTISRDNAKNSLYLQMNSLRAEDTAVYYCARRGLLLDYWGQGTTVTVSS

SEQ ID NO: 71 - protein, 515 aa, synthetic construct
  REGION 1..515: note = synthetic polypeptide
DIQMTQSPSSLSASLGDRATITCRASKTVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGVVQPGGSLRLSCAASGFTFSNYGMHWVRQAPEKGLEWVSYISSGSSTIYYADSVKGRFTISRDNSKNTLYLQMNSLRAEDTAVYYCARRGLLLDYWGQGTTVTVSSASTKGPSVFPLAPLESSGSDIQMTQSPSSLSASLGDRATITCRASKTVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGVVQPGGSLRLSCAASGFTFSNYGMHWVRQAPEKGLEWVSYISSGSSTIYYADSVKGRFTISRDNSKNTLYLQMNSLRAEDTAVYYCARRGLLLDYWGQGTTVTVSS

SEQ ID NO: 72 - protein, 515 aa, synthetic construct
  REGION 1..515: note = synthetic polypeptide
DIQMTQSPSSLSASLGDRATITCRASKTVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGDVKPGGSLRLSCAASGFTFSNYGMHWVRQAPEKGLEWVSYISSGSSTIYYADSVKGRFTISRDNSKNTLYLQMNSLRAEDTAVYYCARRGLLLDYWGQGTTVTVSSASTKGPSVFPLAPLESSGSDIQMTQSPSSLSASLGDRATITCRASKTVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGDVKPGGSLRLSCAASGFTFSNYGMHWVRQAPEKGLEWVSYISSGSSTIYYADSVKGRFTISRDNSKNTLYLQMNSLRAEDTAVYYCARRGLLLDYWGQGTTVTVSS

SEQ ID NO: 73 - protein, 515 aa, synthetic construct
  REGION 1..515: note = synthetic polypeptide
DIQMTQSPSSLSASVGDRVTITCRASKTVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGLVQPGGSLRLSCAASGFTFSNYGMHWVRQAPGKGLEWVSYISSGSSTIYYADSVKGRFTISRDNAKNSLYLQMNSLRAEDTAVYYCARRGLLLDYWGQGTTVTVSSASTKGPSVFPLAPLESSGSDIQMTQSPSSLSASVGDRVTITCRASKTVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGLVQPGGSLRLSCAASGFTFSNYGMHWVRQAPGKGLEWVSYISSGSSTIYYADSVKGRFTISRDNAKNSLYLQMNSLRAEDTAVYYCARRGLLLDYWGQGTTVTVSS

SEQ ID NO: 74 - protein, 515 aa, synthetic construct
  REGION 1..515: note = synthetic polypeptide
DIQMTQSPSSLSASVGDRVTITCRASKTVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGVVQPGGSLRLSCAASGFTFSNYGMHWVRQAPEKGLEWVSYISSGSSTIYYADSVKGRFTISRDNSKNTLYLQMNSLRAEDTAVYYCARRGLLLDYWGQGTTVTVSSASTKGPSVFPLAPLESSGSDIQMTQSPSSLSASVGDRVTITCRASKTVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGVVQPGGSLRLSCAASGFTFSNYGMHWVRQAPEKGLEWVSYISSGSSTIYYADSVKGRFTISRDNSKNTLYLQMNSLRAEDTAVYYCARRGLLLDYWGQGTTVTVSS

SEQ ID NO: 75 - protein, 515 aa, synthetic construct
  REGION 1..515: note = synthetic polypeptide
DIQMTQSPSSLSASVGDRVTITCRASKTVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGDVKPGGSLRLSCAASGFTFSNYGMHWVRQAPEKGLEWVSYISSGSSTIYYADSVKGRFTISRDNSKNTLYLQMNSLRAEDTAVYYCARRGLLLDYWGQGTTVTVSSASTKGPSVFPLAPLESSGSDIQMTQSPSSLSASVGDRVTITCRASKTVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGDVKPGGSLRLSCAASGFTFSNYGMHWVRQAPEKGLEWVSYISSGSSTIYYADSVKGRFTISRDNSKNTLYLQMNSLRAEDTAVYYCARRGLLLDYWGQGTTVTVSS

SEQ ID NO: 76 - protein, 515 aa, synthetic construct
  REGION 1..515: note = synthetic polypeptide
DIQMTQSPSSLSASLGDRATITCRASKTVSTSSYSYMHWYQQKPGQAPKLLIKYASYLESGVPSRFSGSGSGTDFTLTISSLQPEDFATYYCQHSREFPWTFGQGTKVEIKRADAAPGGGGSGGGGSGGGGSEVQLVESGGGLVQPGGSLRLSCAASGFTFSNYGMHWVRQAPGKGLEWVSYISSGSSTIYYADSVKGRFTISRDNAKNSLYLQMNSLRAEDTAVYYCARRGLLLDYWGQGTTVTVSSASTKGPSVFPLAPLESSGSDIQMTQSPSSLSASLGDRATITCRASKTVSTSSYSYMHWYQQKPGQAPKLLIKYASYLESGVPSRFSGSGSGTDFTLTISSLQPEDFATYYCQHSREFPWTFGQGTKVEIKRADAAPGGGGSGGGGSGGGGSEVQLVESGGGLVQPGGSLRLSCAASGFTFSNYGMHWVRQAPGKGLEWVSYISSGSSTIYYADSVKGRFTISRDNAKNSLYLQMNSLRAEDTAVYYCARRGLLLDYWGQGTTVTVSS

SEQ ID NO: 77 - protein, 446 aa, synthetic construct
  REGION 1..446: note = synthetic polypeptide
EVQLVESGGGLVQPGGSLRLSCAASGFTFSNYGMHWVRQAPGKGLEWVSYISSGSSTIYYADSVKGRFTISRDNAKNSLYLQMNSLRAEDTAVYYCARRGLLLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 78 - protein, 98 aa, synthetic construct
  REGION 1..98: note = synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 79 - protein, 12 aa, synthetic construct
  REGION 1..12: note = synthetic polypeptide
EPKSCDKTHTCP

SEQ ID NO: 80 - protein, 113 aa, synthetic construct
  REGION 1..113: note = synthetic polypeptide
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 81 - protein, 107 aa, synthetic construct
  REGION 1..107: note = synthetic polypeptide
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 82 - protein, 446 aa, synthetic construct
  REGION 1..446: note = synthetic polypeptide
EVQLVESGGGLVQPGGSLRLSCAASGFTFSNYGMHWVRQAPGKGLEWVSYISSGSSTIYYADSVKGRFTISRDNAKNSLYLQMNSLRAEDTAVYYCARRGLLLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYDSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 83 - protein, 113 aa, synthetic construct
  REGION 1..113: note = synthetic polypeptide
PCPAPELLGGPSVFLFPPKPKDTLMISRTPEVTCVVVDVSHEDPEVKFNWYVDGVEVHNAKTKPREEQYDSTYRVVSVLTVLHQDWLNGKEYKCKVSNKALPAPIEKTISKAK

SEQ ID NO: 84 - protein, 446 aa, synthetic construct
  REGION 1..446: note = synthetic polypeptide
EVQLVESGGGLVQPGGSLRLSCAASGFTFSNYGMHWVRQAPGKGLEWVSYISSGSSTIYYADSVKGRFTISRDNAKNSLYLQMNSLRAEDTAVYYCARRGLLLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYDSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 85 - protein, 113 aa, synthetic construct
  REGION 1..113: note = synthetic polypeptide
PCPAPEAAGGPSVFLFPPKPKDTLMISRTPEVTCVVVDVSHEDPEVKFNWYVDGVEVHNAKTKPREEQYDSTYRVVSVLTVLHQDWLNGKEYKCKVSNKALPAPIEKTISKAK

SEQ ID NO: 86 - protein, 113 aa, synthetic construct
  REGION 1..113: note = synthetic polypeptide
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 87 - protein, 218 aa, synthetic construct
  REGION 1..218: note = synthetic polypeptide
DIQMTQSPSSLSASLGDRATITCRASKTVSTSSYSYMHWYQQKPGQPPKLLIKYASYLESGVPSRFSGSGSGTDFTLTISSLQPEDAATYYCQHSREFPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 88 - protein, 242 aa, synthetic construct
  REGION 1..242: note = synthetic polypeptide
DIVLTQSPASLAVSPGQRATITCRASKSVSTSSYSYMHWYQQKPGQPPKLLIYYASYLESGVPARFSGSGSGTDFTLTINPVEANDTANYYCQHSREFPWTFGQGTKVEIKGGGGSGGGGSGGGGSEVQLVESGGGLVQPGGSLRLSCSASGFTFSNYGMHWVRQAPGKGLEYVSYISSGSSTIYYADTVKGRFTISRDNSKNTLYLQMSSLRAEDTAVYYCVKRGLLLDYWGQGTLVTVSS

SEQ ID NO: 89 - protein, 8 aa, synthetic construct
  REGION 1..8: note = synthetic polypeptide
GFTFSDYG

SEQ ID NO: 90 - protein, 7 aa, synthetic construct
  REGION 1..7: note = synthetic polypeptide
YASYLQS

SEQ ID NO: 91 - protein, 15 aa, synthetic construct
  REGION 1..15: note = synthetic polypeptide
RASKSVSTSSYSYLA

(19) United States
(12) Patent Application Publication
(10) Pub. No.: US 2026/0265414 A1
(43) Pub. Date: Sep. 10, 2026

(54) COMPOSITIONS AND METHODS OF USING CELL-PENETRATING ANTIBODIES IN COMBINATION WITH IMMUNE CHECKPOINT MODULATORS

(71) Applicant: Yale University, New Haven, CT (US)

(72) Inventors: Peter Glazer, Guilford, CT (US); Audrey Turchick, Chelmsford, MA (US)

(21) Appl. No.: 19/313,360

(22) Filed: Aug. 28, 2025

Related U.S. Application Data
(63) Continuation of application No. 17/272,124, filed on Aug. 30, 2019.
(60) Provisional application No. 62/725,937, filed on Aug. 31, 2018.

(51) Int. Cl. C07K 16/44; A61K 45/06; C07K 16/28

(52) U.S. Cl. CPC C07K 16/44; A61K 45/06; C07K 16/2818; C07K 16/2827; C07K 2317/10; C07K 2317/24; C07K 2317/33; C07K 2317/565; C07K 2317/72; C07K 2317/82

(57) ABSTRACT

Combination therapies including administering a subject in need thereof a cell-penetrating binding protein, such as an antibody, and an immune checkpoint modulator are provided. Typically, the cell-penetrating binding protein can induce DNA damage or reduce DNA damage repair in an effective amount to activate the innate immunity inflammatory pathway in target cells such as cancer cells or infected cells. For example, in some embodiments, the cell-penetrating binding protein increases induced p21 and/or p27 protein expression, increases accumulation of single-strand DNA in the cytosol, increases phosphorylation of STAT1, or a combination thereof in target cells. The subject can have cancer or an infection and the combination of the cell-penetrating binding protein and the immune checkpoint modulator reduce one or more symptoms of cancer or infection, preferably to a greater degree than administering the subject the same amount of cell-penetrating binding protein alone or the same amount of immune checkpoint modulator alone.

### CROSS-REFERENCE TO RELATED APPLICATIONS

This application is a continuation of U.S. Ser. No. 17/272,124, filed Feb. 26, 2021, which is a National Stage Entry of PCT/US2019/048954 filed Aug. 30, 2019, which claims the benefit of and priority to U.S. Ser. No. 62/725,937, filed Aug. 31, 2018, each of which are incorporated by reference herein in their entireties.

### STATEMENT REGARDING FEDERALLY SPONSORED RESEARCH

This invention was made with government support under Grant No. CA197574 awarded by National Institutes of Health. The government has certain rights in the invention.

### REFERENCE TO THE SEQUENCE LISTING

The Sequence Listing submitted as a XML file named “YU_7450_CON_US” created on May 18, 2026, and having a size of 128,331 bytes is hereby incorporated by reference pursuant to 37 C.F.R. § 1.834 (c) (1).

### FIELD OF THE INVENTION

The invention is generally directed to combination therapies including a cell-penetrating binding protein and an immune checkpoint modulator, and method of use thereof particularly for the treatment of cancer.

### BACKGROUND OF THE INVENTION

GMP-AMP (cGAMP) synthase (cGAS) is a cytosolic DNA sensor that activates innate immune responses through production of the second messenger cGAMP. In turn, cGAMP activates the adaptor STING (Chen, et al., Nat Immunol (2016) 17 (10): 1142-9.10.1038/ni.3558). The cGAS-STING pathway not only mediates protective immune defense against infection by a large variety of DNA-containing pathogens (e.g., microbial DNA) but also detects tumor-derived DNA and generates intrinsic antitumor immunity. The STING pathway, and its role immune modulation and cancer develop are reviewed in, for example, Corrales, et al., Cell Res (2017) 27 (1): 96-108.10.1038/cr.2016; Corrales, et al., J Clin Invest (2016) 126 (7): 2404-11.10.1172/JCI86892; Rivera Vargas, et al., Eur J Cancer (2017) 75:86-97.10.1016/j.ejca.2016.1; Qiao, et al., Curr Opin Immunol (2017) 45:16-20.10.1016/j.coi.2016.12.005; He, et al., Cancer Lett (2017) 402:203-12.10.1016/j.canlet.2017.05.026

In the tumor microenvironment, T cells, endothelial cells, and fibroblasts, stimulated with STING agonists ex vivo produce type-I IFNs (Corrales, et al., Cell Rep (2015) 11 (7): 1018-30.10.1016/j.celrep.2015.04.031). By contrast, most studies indicated that tumor cells can inhibit STING pathway activation, potentially leading to immune evasion during carcinogenesis (He, et al., Cancer Lett (2017) 402:203-12.10.1016/j.canlet.2017.05.026; Xia, et al., Cancer Res (2016) 76 (22): 6747-59.10.1158/0008-5472.CAN-16-1404). For example, evidence shows that activation of the STING pathway correlates with the induction of a spontaneous antitumor T-cell response involving the expression of type-I IFN genes (Chen, et al., Nat Immunol (2016) 17 (10): 1142-9.10.1038/ni.3558; Barber, et al., Nat Rev Immunol (2015) 15 (12): 760-70.10.1038/nri3921; Woo, et al., Immunity (2014) 41 (5): 830-42.10.1016/j.immuni.2014.10.017). Furthermore, host STING pathway is required for efficient cross-priming of tumor-Ag specific CD8+ T cells mediated by DCs (Woo, et al., Immunity (2014) 41 (5): 830-42.10.1016/j.immuni.2014.10.017; Deng, et al., Immunity (2014) 41 (5): 843-52.10.1016/j.immuni.2014.10.019). Based on these results, direct pharmacologic stimulation of the STING pathway has been explored as a cancer therapy.

Additionally, strategies that combine STING immunotherapy with other immunomodulatory agents are being explored. The antitumor efficacy of cGAMP administered by i.t. injection into B16.F10 tumors was enhanced when combined with anti-programmed death-1 (PD-1) and anti-cytotoxic T-lymphocyte associated-4 (CTLA-4) antibodies (Demaria, et al., Proc Natl Acad Sci USA (2015) 112 (50): 15408-13.10.1073/pnas.1512832112). In other studies, CDNs together with anti-PD-1 incited much stronger antitumor effects than monotherapy in a mouse model of squamous cell carcinoma model as well as of melanoma (Gadkaree, et al., Head Neck (2017) 39 (6): 1086-94.10.1002/hed.24704; Wang, et al., Proc Natl Acad Sci USA (2017) 114 (7): 1637-42.10.1073/pnas.1621363114). Luo et al. showed encouraging results by combining a STING-activating nanovaccine and an anti-PD1 antibody, which lead to generation of long-term antitumor memory in TC-1 tumor model (Luo, et al., Nat Nanotechnol (2017) 12 (7): 648-54.10.1038/nnano.2017.52).

STING agonists can also enhance antitumor responses when combined with tumor vaccines. CDN ligands formulated with granulocyte-macrophage colony-stimulating factor-producing cellular cancer vaccines, termed STINGVAX, showed strong in vivo therapeutic efficacy in several established cancer models (Fu, et al., Sci Transl Med (2015) 7 (283): 283ra52.10.1126/scitranslmed.aaa4306), and STING agonists in combination with traditional chemotherapeutic agents or radiotherapy can trigger an antitumor response (Xia, et al., Cancer Res (2016) 76 (22): 6747-59.10.1158/0008-5472.CAN-16-1404; Baird, et al., Cancer Res (2016) 76 (1): 50-61.10.1158/0008-5472.CAN-14-3619).

Despite these gains, improved compositions and methods for treating cancer remain desirable.

Thus, it is an object of the present disclosure of the invention to provide improved compositions and methods of use thereof for treating cancer.

### SUMMARY OF THE INVENTION

It has been discovered that cell-penetrating binding proteins disclosed herein can activate innate immunity. For example, in some embodiments, the cell-penetrating binding proteins act as cGAS/STING pathway agonists. Such binding proteins may therefore be particularly useful when used in combination with immune checkpoint modulators. Accordingly, combination therapies that including administering a subject in need thereof a cell-penetrating binding protein and an immune checkpoint modulator, and compositions for use therein are provided. In some embodiments, the cell-penetrating binding protein can induce DNA damage or reduce DNA damage repair in an effective amount to activate the cGAS/STING inflammatory pathway in target cells such as cancer cells or infected cells. For example, in some embodiments, the cell-penetrating binding protein increases induced p21 and/or p27 protein expression, increases accumulation of single-strand DNA in the cytosol, increases phosphorylation of STAT1, or a combination thereof in target cells. Typically, the binding protein increases the presence of phosphorylated STAT1 in the cells. In some embodiments, this increase in phosphorylated STAT1 is not cGAS-dependent. In some embodiments, cGAS protein level is the same or similar to untreated cells. In some embodiments, the cell-penetrating binding protein is a cell-penetrating antibody.

In preferred embodiments, the subject has cancer or an infection, and the combination of the cell-penetrating binding protein and the immune checkpoint modulator reduce one or more symptoms of cancer or infection, preferably to a greater degree than administering to the subject the same amount of cell-penetrating binding protein alone or the same amount of immune checkpoint modulator alone. In some embodiments, the reduction in the one or more symptoms is a more than the additive reduction compared to the reduction achieved by administering the cell-penetrating binding protein and/or the immune checkpoint modulator individually and in the absence of the other. In some embodiments, cells associated with the cancer (e.g., cancer cells) or infection (e.g., infected cells) are DNA damage repair deficient.

The cell-penetrating binding protein and the immune checkpoint inhibitor can be administered in the same or different pharmaceutical compositions, and at the same or different times. Thus, pharmaceutical compositions including a cell-penetrating binding protein, an immune checkpoint modulator, and a combination thereof are also provided. In some embodiments, the cell-penetrating binding protein is administered to the subject 1, 2, 3, 4, 5, 6, 8, 10, 12, 18, or 24 hours, 1, 2, 3, 4, 5, 6, or 7 days, 1, 2, 3, or 4 weeks, or any combination thereof prior to administration of the immune checkpoint modulator to the subject. In some embodiments, the immune checkpoint modulator is administered to the subject 1, 2, 3, 4, 5, 6, 8, 10, 12, 18, or 24 hours, 1, 2, 3, 4, 5, 6, or 7 days, 1, 2, 3, or 4 weeks, or any combination thereof prior to administration of the cell-penetrating binding protein to the subject.

In some embodiments, the subject is administered one or more additional active agents, for example, a chemotherapeutic agent, an anti-infective agent, and combinations thereof; treated with radiation; operated upon (e.g., surgery); or any combination thereof. Exemplary cell-penetrating binding proteins are also provided. In some embodiments, the binding protein can penetrate the cell, penetrate the nucleus, or a combination thereof without the aid of a cell-penetrating conjugate or carrier. The cell-penetrating binding protein can be an anti-DNA antibody, for example, an anti-DNA antibody derived from a subject with, or an animal model of, an autoimmune disease such as systemic lupus erythematous. In some embodiments, the cell-penetrating binding protein inhibits RAD51. In some embodiments, the cell-penetrating binding protein hydrolyzes DNA.

Preferred binding proteins include 3E10 monoclonal antibody and cell-penetrating fragments and fusion proteins thereof, as well as humanized forms and other variants thereof. The binding proteins and be antibodies. For example, in some embodiments, the cell-penetrating binding protein, such as an antibody, includes (i) the CDRs of SEQ ID NO:6 or 7 and SEQ ID NO:1 or 2, or a humanized form thereof; (ii) a heavy chain having an amino acid sequence having at least 85% sequence identity to SEQ ID NO:6 or 7; and a light chain having an amino acid sequence having at least 85% sequence identity to SEQ ID NO:1 or 2; (iii) the CDRs of SEQ ID NO:6 or 7 and SEQ ID NO:1 or 2, or a humanized forms thereof; or (iv) a heavy chain having an amino acid sequence including at least 85% sequence identity to SEQ ID NO:6 or 7; and a light chain having an amino acid sequence including at least 85% sequence identity to SEQ ID NO:1 or 2. The cell-penetrating binding protein, such as an antibody, can have the same or different epitope specificity as monoclonal antibody 3E10, produced by ATCC Accession No. PTA 2439 hybridoma. The antibody can be a recombinant antibody having the paratope of monoclonal antibody 3E10.

Other binding proteins include 5C6 monoclonal antibody and cell-penetrating fragments and fusion proteins thereof, as well as humanized forms and other variants thereof. For example, in some embodiments, the cell-penetrating binding protein, such as an antibody, include (i) the CDRs of SEQ ID NO:16 and SEQ ID NO:12, or a humanized form thereof; (ii) a heavy chain including an amino acid sequence including at least 85% sequence identity to SEQ ID NO:16; and a light chain including an amino acid sequence including at least 85% sequence identity to SEQ ID NO:12; (iii) the CDRs of SEQ ID NO:16 and SEQ ID NO:12, or a humanized forms thereof; or (iv) a heavy chain including an amino acid sequence including at least 85% sequence identity to SEQ ID NO:16; and a light chain including an amino acid sequence including at least 85% sequence identity to SEQ ID NO:12.

Fragments and fusion proteins are also provided. For example, in some embodiments, the antibody is a monovalent, divalent, or multivalent single chain variable fragment (scFv), diabody; or humanized form or variant thereof.

Exemplary immune checkpoint modulators are also provided. Typically, the immune checkpoint modulator induces an immune response against the cancer or infection. In some embodiments, the immune checkpoint modulator reduces an immune inhibitory pathway. In some embodiments, the immune checkpoint modulator increases an immune stimulatory pathway.

A preferred immune inhibitory pathway is the PD-1 pathway. Thus, in some embodiments, the immune checkpoint modulator is a PD-1 antagonist or a PD-1 ligand antagonist. In other embodiments, the immune checkpoint inhibitor is a CTLA4 antagonist. In some embodiments, the immune checkpoint modulator is an antibody, for example an inhibitory or blocking antibody.

In other embodiments, the immune checkpoint modulator is a CAR-T cell. In other embodiments, the immune checkpoint modulator is an oncolytic virus.

In an embodiment, the present disclosure encompasses a method of treating cancer or an infection including administering to a subject in need thereof an effective amount of the combination of a cell-penetrating binding protein that induces or increase DNA damage or reduces or impairs DNA damage repair, or a combination thereof; and an immune checkpoint modulator that induces, increases, or enhances an immune response. In some embodiments, administration of the combination to a subject in need thereof results in a more than additive reduction in one or more symptoms of cancer or infection compared to the reduction achieved by administering the cell-penetrating binding protein, such as an antibody, or the immune checkpoint modulator individually and in the absence of the other. In some embodiments, the cells associated with the cancer or infection are DNA damage repair deficient. In another embodiment, the cell-penetrating binding protein, such as an antibody, is administered to the subject 1, 2, 3, 4, 5, 6, 8, 10, 12, 18, or 24 hours, 1, 2, 3, 4, 5, 6, or 7 days, 1, 2, 3, or 4 weeks, or any combination thereof prior to administration of the immune checkpoint modulator to the subject.

In another embodiment, the immune checkpoint modulator is administered to the subject 1, 2, 3, 4, 5, 6, 8, 10, 12, 18, or 24 hours, 1, 2, 3, 4, 5, 6, or 7 days, 1, 2, 3, or 4 weeks, or any combination thereof prior to administration of the cell-penetrating binding protein, such as an antibody, to the subject. In another embodiment, the cell-penetrating binding protein and the immune checkpoint modulator are administered sequentially.

In another embodiment, the method further includes administering to the subject one or more additional active agents selected from the group consisting of a chemotherapeutic agent, an anti-infective agent, and combinations thereof. In another embodiment, the method may further include surgery or radiation therapy.

In another embodiment, the cell-penetrating binding protein can penetrate the cell, penetrate the nucleus, or a combination thereof without the aid of a conjugate or carrier. For example, the cell-penetrating binding protein can be naked.

In another embodiment, the cell-penetrating binding protein is an anti-DNA antibody.

In some embodiments, the anti-DNA antibody is derived from a subject with or an animal model of an autoimmune disease. In some embodiments, the autoimmune disease is systemic lupus erythematous. In some embodiments, the cell-penetrating binding protein inhibits RAD51. In some embodiments, the cell-penetrating binding protein includes a 3E10 monoclonal antibody or a cell-penetrating fragment thereof; a monovalent, divalent, or multivalent single chain variable fragment (scFv); or a diabody; or humanized form or variant thereof. In some embodiments, the cell-penetrating binding protein includes (i) the CDRs of SEQ ID NO:6 or 7 and SEQ ID NO:1 or 2, or a humanized form thereof; (ii) a heavy chain including an amino acid sequence including at least 85% sequence identity to SEQ ID NO:6 or 7; and a light chain including an amino acid sequence including at least 85% sequence identity to SEQ ID NO:1 or 2; (iii) the CDRs of SEQ ID NO:6 or 7 and SEQ ID NO:1 or 2, or a humanized forms thereof; or (iv) a heavy chain including an amino acid sequence including at least 85% sequence identity to SEQ ID NO:6 or 7; and a light chain including an amino acid sequence including at least 85% sequence identity to SEQ ID NO:1 or 2. In some embodiments, the cell-penetrating binding protein includes the same or different epitope specificity as monoclonal antibody 3E10, produced by ATCC Accession No. PTA 2439 hybridoma. In another embodiment, the cell-penetrating binding protein includes one of the following combinations of CDRs:
SEQ ID NO:30, SEQ ID NO:31, SEQ ID NO:33 and SEQ ID NO:34, SEQ ID NO: 36, SEQ ID NO:37;
SEQ ID NO:30, SEQ ID NO:32, SEQ ID NO:33 and SEQ ID NO:34, SEQ ID NO: 36, SEQ ID NO:37;
SEQ ID NO:30, SEQ ID NO:32, SEQ ID NO:33 and SEQ ID NO:34, SEQ ID NO: 36, SEQ ID NO:37; or,
SEQ ID NO:30, SEQ ID NO:32, SEQ ID NO:33 and SEQ ID NO:35, SEQ ID NO: 36, SEQ ID NO:37.

In another embodiment, the cell-penetrating binding protein is a recombinant antibody having the paratope of monoclonal antibody 3E10.

In some embodiments, the cell-penetrating antibody hydrolyzes DNA. In another embodiment, the cell-penetrating binding protein includes a 5C6 monoclonal antibody or a cell-penetrating fragment thereof; a monovalent, divalent, or multivalent single chain variable fragment (scFv); or a diabody; or humanized form or variant thereof. In another embodiment, the cell-penetrating binding protein includes (i) the CDRs of SEQ ID NO:16 and SEQ ID NO:12, or a humanized form thereof; (ii) a heavy chain including an amino acid sequence including at least 85% sequence identity to SEQ ID NO:16; and a light chain including an amino acid sequence including at least 85% sequence identity to SEQ ID NO: 12; (iii) the CDRs of SEQ ID NO:16 and SEQ ID NO:12, or a humanized forms thereof; or (iv) a heavy chain including an amino acid sequence including at least 85% sequence identity to SEQ ID NO:16; and a light chain including an amino acid sequence including at least 85% sequence identity to SEQ ID NO:12.

In another embodiments, the methods of treating cancer include administering to a subject in need thereof an effective amount of the combination of a cell-penetrating anti-DNA binding protein which includes:
a V_{H} including an amino acid sequence as shown in any one of SEQ ID NOs: 9, 11, or 45 to 52 and a V_{L} including an amino acid sequence as shown in any one of SEQ ID NOs: 3 to 5, or 53 to 58; or, an amino acid sequence as shown in any one of SEQ ID NOs: 61-76; and,
an immune checkpoint modulator which is an anti-PD1 an anti-PDL1, or an anti-CTLA4 antibody, wherein administration of the combination to a subject in need thereof reduces one or more symptoms of cancer to a greater degree than administering to the subject the same amount of the cell-penetrating anti-DNA binding protein alone or the same amount of the immune checkpoint modulator alone.

In a particular embodiment, the cell-penetrating anti-DNA binding protein includes a V_{H} including an amino acid sequence as shown in SEQ ID NO:50 and a V_{L} including an amino acid sequence as shown in SEQ ID NO:56. In another embodiment, the cell-penetrating anti-DNA binding protein includes an amino acid sequence as shown in SEQ ID NO: 70. In another embodiment, the cell-penetrating anti-DNA binding protein is an antibody.

### BRIEF DESCRIPTION OF THE DRAWING

FIG. 1A is a bar graph showing normalized quantification of p21 and p27 protein expression, as determined via western blot, upon treatment of cancer cells with 1 μM 3E10.

FIG. 1B is a bar graph showing normalized quantification of phosphorylated STAT1 protein expression, as determined via western blot, upon treatment of cancer cells with 1 μM 3E10.

FIGS. 2A-2C are plots illustrating quantification of western blots analysis of phosphorylation status of STAT1 (pSTAT1) and cGAS protein levels in B16 (2A), MC38 (2B), and MB231 (2C) cells following treatment with full-length 3E10 and cGAS targeting siRNA.

FIGS. 3A-3B are plots illustrating quantification of western blots analysis of phosphorylation status of STAT1 (pSTAT1) and cGAS protein levels in cGas-deficient U251 cell (3A) and MCF10A cGAS-knockout (KO) cells (3B) following treatment with full-length 3E10.

### DETAILED DESCRIPTION OF THE INVENTION

### I. Definitions

The term “anti-DNA binding protein” is used in the context of the present disclosure to refer to proteins capable of binding DNA. In some embodiments, anti-DNA binding proteins bind DNA and impair DNA repair. Exemplary binding proteins include immunoglobulin, antibodies and antigenic binding fragments such as scFv and di-scFv. Other examples of binding proteins are discussed below.

As used herein, the term “single chain Fv” or “scFv” as used herein means a single chain variable fragment that includes a light chain variable region (V_{L}) and a heavy chain variable region (V_{H}) in a single polypeptide chain joined by a linker which enables the scFv to form the desired structure for antigen binding (i.e., for the V_{H} and V_{L} of the single polypeptide chain to associate with one another to form a Fv). The V_{L} and V_{H} regions may be derived from the parent antibody or may be chemically or recombinantly synthesized.

As used herein, the term “variable region” is intended to distinguish such domain of the immunoglobulin from domains that are broadly shared by antibodies (such as an antibody Fc domain). The variable region includes a “hypervariable region” whose residues are responsible for antigen binding. The hypervariable region includes amino acid residues from a “Complementarity Determining Region” or “CDR” (i.e., typically at approximately residues 24-34 (L1), 50-56 (L2) and 89-97 (L3) in the light chain variable domain and at approximately residues 27-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, MD. (1991)) and/or those residues from a “hypervariable loop” (i.e., residues 26-32 (L1), 50-52 (L2) and 91-96 (L3) in the light chain variable domain and 26-32 (H1), 53-55 (H2) and 96-101 (H3) in the heavy chain variable domain; Chothia and Lesk, 1987, J. Mol. Biol. 196:901-917).

As used herein, the term “Framework Region” or “FR” residues are those variable domain residues other than the hypervariable region residues as herein defined.

As used herein, the term “antibody” refers to natural or synthetic antibodies that bind a target antigen. The term includes polyclonal and monoclonal antibodies. In addition to intact immunoglobulin molecules, also included in the term “antibodies” are fragments or polymers of those immunoglobulin molecules, and human or humanized versions of immunoglobulin molecules that bind the target antigen.

As used herein, an “antigen binding fragment” of an antibody includes one or more variable regions of an intact antibody. Examples of antibody fragments include Fab, Fab′, F(ab′)2 and Fv fragments; diabodies; linear antibodies; single-chain antibody molecules and multispecific antibodies formed from antibody fragments. For example, the term antigen binding fragment may be used to refer to recombinant single chain Fv fragments (scFv) as well as divalent (di-scFv) and trivalent (tri-scFV) forms thereof.

As used herein, the term “cell-penetrating antibody” refers to an immunoglobulin protein, antigen binding fragment, or molecule thereof that is transported into the cytoplasm and/or nucleus of living mammalian cells. Accordingly, the term “cell-penetrating binding protein” can be used in the context of the present disclosure to encompass these molecules. In some embodiments the cell-penetrating binding protein binds DNA (i.e. it is an “anti-DNA binding protein). The term “cell-penetrating anti-DNA antibody” refers to an antibody, or antigen binding fragment or molecule thereof that is transported into the cytoplasm and/or nucleus of living mammalian cells and binds DNA (e.g., single-stranded and/or double-stranded DNA). Again, the term “cell-penetrating anti-DNA binding protein” can be used in the context of the present disclosure to encompass these molecules. In some embodiments, a cell-penetrating anti-DNA antibody is transported into the cytoplasm and/or nucleus of a cell without the aid of a carrier or conjugate. In another embodiment, a cell-penetrating anti-DNA antibody is conjugated to a cell-penetrating moiety, such as a cell-penetrating peptide. One of skill in the art will appreciate that the term “cell-penetrating” can be used in the context of the present disclosure to refer to other compounds that penetrate cells. For example, the term can be used to refer more specifically to a scFv that is transported into the nucleus of a cell without the aid of a carrier or conjugate and binds DNA (e.g., single-stranded and/or double-stranded DNA).

As used herein, an “antigen binding fragment” of an antibody includes one or more variable regions of an intact antibody. Examples of antibody fragments include Fab, Fab′, F(ab′)2 and Fv fragments; diabodies; linear antibodies; single-chain antibody molecules and multispecific antibodies formed from antibody fragments. For example, the term antigen binding fragment may be used to refer to recombinant single chain Fv fragments (scFv) as well as divalent (di-scFv) and trivalent (tri-scFV) forms thereof.

Such fragments can be produced via various methods known in the art. For example, di-scFv encompassed by the present disclosure can be produced and purified.

As used herein, the terms “full-length antibody”, “intact antibody” or “whole antibody” are used interchangeably to refer to an antibody in its substantially intact form, as opposed to an antigen binding fragment of an antibody. Specifically, whole antibodies include those with heavy and light chains including an Fc region. The constant domains may be wild-type sequence constant domains (e.g., human wild-type sequence constant domains) or amino acid sequence variants thereof.

As used herein, “variable region” refers to the portions of the light and/or heavy chains of an antibody as defined herein that specifically binds to an antigen and, for example, includes amino acid sequences of CDRs; i.e., CDR1, CDR2, and CDR3, and framework regions (FRs). For example, the variable region includes three or four FRs (e.g., FR1, FR2, FR3 and optionally FR4) together with three CDRs. V_{H} refers to the variable region of the heavy chain. V_{L} refers to the variable region of the light chain.

As used herein, the term “complementarity determining regions” (syn. CDRs; i.e., CDR1, CDR2, and CDR3) refers to the amino acid residues of an antibody variable region the presence of which are major contributors to specific antigen binding. Each variable region typically has three CDR regions identified as CDR1, CDR2 and CDR3. In one example, the amino acid positions assigned to CDRs and FRs are defined according to Kabat Sequences of Proteins of Immunological Interest, National Institutes of Health, Bethesda, Md., 1987 and 1991 (also referred to herein as “the Kabat numbering system” or “Kabat”.

Other conventions that include corrections or alternate numbering systems for variable domains include IMGT (Lefranc, et al. (2003), Dev Comp Immunol 27:55-77), Chothia (Chothia C, Lesk A M (1987), J Mal Biol 196:901-917; Chothia, et al. (1989), Nature 342:877-883) and AHo (Honegger A, Plückthun A (2001) J Mol Biol 309:657-670). For convenience, examples of binding proteins of the present disclosure may also be labelled according to IMGT. These examples are expressly indicated as such. For example, see SEQ ID NO: 37-44

As used herein “framework regions” (Syn. FR) are those variable domain residues other than the CDR residues.

As used herein, the term “constant region” as used herein, refers to a portion of heavy chain or light chain of an antibody other than the variable region. In a heavy chain, the constant region generally includes a plurality of constant domains and a hinge region, e.g., a IgG constant region includes the following linked components, a constant heavy CH1, a linker, a CH2 and a CH3. In a heavy chain, a constant region includes a Fc. In a light chain, a constant region generally include one constant domain (a CL1).

As used herein, the term “fragment crystalizable” or “Fc” or “Fc region” or “Fc portion” (which can be used interchangeably herein) refers to a region of an antibody including at least one constant domain and which is generally (though not necessarily) glycosylated and which is capable of binding to one or more Fc receptors and/or components of the complement cascade. The heavy chain constant region can be selected from any of the five isotypes: α, δ, ε, γ, or μ. Exemplary heavy chain constant regions are gamma 1 (IgG1), gamma 2 (IgG2) and gamma 3 (IgG3), or hybrids thereof.

As used herein, a “constant domain” is a domain in an antibody the sequence of which is highly similar in antibodies/antibodies of the same type, e.g., IgG or IgM or IgE. A constant region of an antibody generally includes a plurality of constant domains, e.g., the constant region of γ, α or δ heavy chain includes two constant domains.

As used herein, the term “binds” in reference to the interaction of a binding protein and an antigen means that the interaction is dependent upon the presence of a particular structure (e.g., an antigenic determinant or epitope) on the antigen. For example, a binding protein recognizes and binds to a specific antigen structure rather than to antigens generally. For example, if a binding protein binds to epitope “A”, the presence of a molecule containing epitope “A” (or free, unlabeled “A”), in a reaction containing labeled “A” and the binding protein, will reduce the amount of labeled “A” bound to the binding protein. In some embodiments anti-DNA binding proteins disclosed herein bind to DNA.

As used herein, the term “specifically binds” refers to the binding of a binding protein disclosed herein such as an antibody to its cognate antigen (for example DNA) while not significantly binding to other antigens. Preferably, an antibody “specifically binds” to an antigen with an affinity constant (Ka) greater than about 10⁵ mol⁻¹ (e.g., 10⁶ mol⁻¹, 10⁷ mol⁻¹, 10⁸ mol⁻¹, 10⁹ mol⁻¹, 10¹⁰ mol⁻¹, 10¹¹ mol⁻¹, and 10¹² mol⁻¹ or more) with that second molecule. In some embodiments anti-DNA binding proteins disclosed herein specifically bind to DNA.

As used herein, the term “monoclonal antibody” or “MAb” refers to an antibody obtained from a substantially homogeneous population of antibodies, i.e., the individual antibodies within the population are identical except for possible naturally occurring mutations that may be present in a small subset of the antibody molecules.

As used herein, the term “DNA repair” refers to a collection of processes by which a cell identifies and corrects damage to DNA molecules. Single-strand defects are repaired by base excision repair (BER), nucleotide excision repair (NER), or mismatch repair (MMR).

Double-strand breaks are repaired by non-homologous end joining (NHEJ), microhomology-mediated end joining (MMEJ), or homologous recombination (HR). After DNA damage, cell cycle checkpoints are activated, which pause the cell cycle to give the cell time to repair the damage before continuing to divide. Checkpoint mediator proteins include BRCA1, MDC1, 53BP1, p53, ATM, ATR, CHK1, CHK2, and p21.

As used herein, the term “impaired DNA repair” refers to a state in which a mutated cell or a cell with altered gene expression is incapable of DNA repair or has reduced activity or efficiency of one or more DNA repair pathways or takes longer to repair damage to its DNA as compared to a wild type cell.

As used herein, the term “chemosensitivity” refers to the relative susceptibility of cancer cells to the effects of anticancer drugs. The more chemosensitive a cancer cell is, the less anticancer drug is required to kill that cell.

As used herein, the term “radiosensitivity” refers to the relative susceptibility of cells to the harmful effect of ionizing radiation. The more radiosensitive a cell is, the less radiation that is required to kill that cell. In general, it has been found that cell radiosensitivity is directly proportional to the rate of cell division and inversely proportional to the cell's capacity for DNA repair.

As used herein, the term “radioresistant” refers to a cell that does not die when exposed to clinically suitable dosages of radiation.

As used herein, the term “neoplastic cell” refers to a cell undergoing abnormal cell proliferation (“neoplasia”). The growth of neoplastic cells exceeds and is not coordinated with that of the normal tissues around it. The growth typically persists in the same excessive manner even after cessation of the stimuli, and typically causes formation of a tumor.

As used herein, the term “tumor” or “neoplasm” refers to an abnormal mass of tissue containing neoplastic cells. Neoplasms and tumors may be benign, premalignant, or malignant.

As used herein, the term “cancer” or “malignant neoplasm” refers to a cell that displays uncontrolled growth and division, invasion of adjacent tissues, and often metastasizes to other locations of the body.

As used herein, the term “antineoplastic” refers to a composition, such as a drug or biologic, that can inhibit or prevent cancer growth, invasion, and/or metastasis.

As used herein, the term “anti-cancer moiety” refers to any agent, such as a peptide, protein, nucleic acid, or small molecule, which can be combined with the disclosed anti-DNA antibodies to enhance the anti-cancer properties of the antibodies. The term includes antineoplastic drugs, antibodies that bind and inhibit other therapeutic targets in cancer cells, and substances having an affinity for cancer cells for directed targeting of cancer cells.

As used herein, the term “virally transformed cell” refers to a cell that has been infected with a virus or that has incorporated viral DNA or RNA into its genome. The virus can be an acutely-transforming or slowly-transforming oncogenic virus. In acutely transforming viruses, the viral particles carry a gene that encodes for an overactive oncogene called viral-oncogene (v-one), and the infected cell is transformed as soon as v-one is expressed. In contrast, in slowly-transforming viruses, the virus genome is inserted near a proto-oncogene in the host genome. Exemplary oncoviruses include Human papillomaviruses (HPV), Hepatitis B (HBV), Hepatitis C (HCV), Human T-lymphotropic virus (HTLV), Kaposi's sarcoma-associated herpesvirus (HHV-8), Merkel cell polyomavirus, Epstein-Barr virus (EBV), Human immunodeficiency virus (HIV), and Human cytomegalovirus (CMV).

As used herein, the “virally infected cell” refers to a cell that has been exposed to or infected with a virus or carries viral genetic material, either RNA or DNA. The virus can be an oncogenic virus or a lytic virus or a latent virus and can cause cancer, immunodeficiency, hepatitis, encephalitis, pneumonitis, respiratory illness, or other disease condition. It has previously been shown that retroviruses, specifically HIV, rely in part upon the base excision repair (BER) pathway for integration into host DNA. For example, the ability of 3E10 to impair DNA repair provides a mechanism whereby 3E10 and other anti-DNA antibodies may ameliorate virally caused diseases, in particular, by interfering with DNA repair and thereby by blocking the DNA or RNA metabolism that is part of virus life cycles as well as part of viral infection of a cell.

As used herein, the term “inhibit” means to decrease an activity, response, condition, disease, or other biological parameter. This can include, but is not limited to, the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

As used herein, the term “fusion protein” refers to a polypeptide formed by the joining of two or more polypeptides through a peptide bond formed between the amino terminus of one polypeptide and the carboxyl terminus of another polypeptide or through linking of one polypeptide to another through reactions between amino acid side chains (for example disulfide bonds between cysteine residues on each polypeptide). The fusion protein can be formed by the chemical coupling of the constituent polypeptides or it can be expressed as a single polypeptide from a nucleic acid sequence encoding the single contiguous fusion protein. Fusion proteins can be prepared using conventional techniques in molecular biology to join the two genes in frame into a single nucleic acid sequence, and then expressing the nucleic acid in an appropriate host cell under conditions in which the fusion protein is produced.

As used herein, the term “variant” refers to a polypeptide or polynucleotide that differs from a reference polypeptide or polynucleotide, but retains essential properties. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more modifications (e.g., substitutions, additions, and/or deletions). A substituted or inserted amino acid residue may or may not be one encoded by the genetic code. A variant of a polypeptide may be naturally occurring such as an allelic variant, or it may be a variant that is not known to occur naturally.

Modifications and changes can be made in the structure of the polypeptides of in disclosure and still obtain a molecule having similar characteristics as the polypeptide (e.g., a conservative amino acid substitution). For example, certain amino acids can be substituted for other amino acids in a sequence without appreciable loss of activity. Because it is the interactive capacity and nature of a polypeptide that defines that polypeptide's biological functional activity, certain amino acid sequence substitutions can be made in a polypeptide sequence and nevertheless obtain a polypeptide with like properties.

In making such changes, the hydropathic index of amino acids can be considered. The importance of the hydropathic amino acid index in conferring interactive biologic function on a polypeptide is generally understood in the art. It is known that certain amino acids can be substituted for other amino acids having a similar hydropathic index or score and still result in a polypeptide with similar biological activity. Each amino acid has been assigned a hydropathic index on the basis of its hydrophobicity and charge characteristics. Those indices are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is believed that the relative hydropathic character of the amino acid determines the secondary structure of the resultant polypeptide, which in turn defines the interaction of the polypeptide with other molecules, such as enzymes, substrates, receptors, antibodies, antigens, and cofactors. It is known in the art that an amino acid can be substituted by another amino acid having a similar hydropathic index and still obtain a functionally equivalent polypeptide. In such changes, the substitution of amino acids whose hydropathic indices are within +2 is preferred, those within +1 are particularly preferred, and those within +0.5 are even more particularly preferred.

Substitution of like amino acids can also be made on the basis of hydrophilicity, particularly where the biological functional equivalent polypeptide or peptide thereby created is intended for use in immunological embodiments. The following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); proline (−0.5±1); threonine (−0.4); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent polypeptide. In such changes, the substitution of amino acids whose hydrophilicity values are within +2 is preferred, those within +1 are particularly preferred, and those within +0.5 are even more particularly preferred.

As outlined above, amino acid substitutions are generally based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include (original residue: exemplary substitution): (Ala: Gly, Ser), (Arg: Lys), (Asn: Gln, His), (Asp: Glu, Cys, Ser), (Gln: Asn), (Glu: Asp), (Gly: Ala), (His: Asn, Gln), (Ile: Leu, Val), (Leu: Ile, Val), (Lys: Arg), (Met: Leu, Tyr), (Ser: Thr), (Thr: Ser), (Tip: Tyr), (Tyr: Trp, Phe), and (Val: Ile, Leu). Embodiments of this disclosure thus contemplate functional or biological equivalents of a polypeptide as set forth above. In particular, embodiments of the polypeptides can include variants having about 50%, 60%, 70%, 80%, 90%, 95%, 96%, 97%, 98%, 99%, or more sequence identity to the polypeptide of interest.

As used herein, the term “percent (%) sequence identity” is defined as the percentage of nucleotides or amino acids in a candidate sequence that are identical with the nucleotides or amino acids in a reference nucleic acid sequence, after aligning the sequences and introducing gaps, if necessary, to achieve the maximum percent sequence identity.

Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

For purposes herein, the % sequence identity of a given nucleotides or amino acids sequence C to, with, or against a given nucleic acid sequence D (which can alternatively be phrased as a given sequence C that has or includes a certain % sequence identity to, with, or against a given sequence D) is calculated as follows:

$$100 times the fraction W / Z ,$$

where W is the number of nucleotides or amino acids scored as identical matches by the sequence alignment program in that program's alignment of C and D, and where Z is the total number of nucleotides or amino acids in D. It will be appreciated that where the length of sequence C is not equal to the length of sequence D, the % sequence identity of C to D will not equal the % sequence identity of D to C.

As used herein, the phrase “pharmaceutically acceptable” refers to compositions, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the phrase “pharmaceutically acceptable carrier” refers to pharmaceutically acceptable materials, compositions or vehicles, such as a liquid or solid filler, diluent, solvent or encapsulating material involved in carrying or transporting any subject composition, from one organ, or portion of the body, to another organ, or portion of the body. Each carrier must be “acceptable” in the sense of being compatible with the other ingredients of a subject composition and not injurious to the patient.

As used herein, the phrase “pharmaceutically acceptable salts” is art-recognized, and includes relatively non-toxic, inorganic and organic acid addition salts of compounds. Examples of pharmaceutically acceptable salts include those derived from mineral acids, such as hydrochloric acid and sulfuric acid, and those derived from organic acids, such as ethanesulfonic acid, benzenesulfonic acid, and p-toluenesulfonic acid. Examples of suitable inorganic bases for the formation of salts include the hydroxides, carbonates, and bicarbonates of ammonia, sodium, lithium, potassium, calcium, magnesium, aluminum, and zinc. Salts may also be formed with suitable organic bases, including those that are non-toxic and strong enough to form such salts.

As used herein, the term “individual,” “host,” “subject,” and “patient” are used interchangeably to refer to any individual who is the target of administration or treatment. The subject can be a vertebrate, for example, a mammal. Thus, the subject can be a human or veterinary patient.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

As used herein, the term “therapeutically effective amount” refers to an amount of the composition (e.g., therapeutic agent) that produces some desired effect at a reasonable benefit/risk ratio applicable to any medical treatment. The effective amount may vary depending on such factors as the disease or condition being treated, the particular targeted constructs being administered, the size of the subject, or the severity of the disease or condition. One of ordinary skill in the art may empirically determine the effective amount of a particular compound without necessitating undue experimentation. In some embodiments, the term “effective amount” refers to an amount of a therapeutic agent or prophylactic agent to reduce or diminish the symptoms of one or more diseases or disorders of the brain, such as reducing tumor size (e.g., tumor volume) or reducing or diminishing one or more symptoms of a neurological disorder, such as memory or learning deficit, tremors or shakes, etc. In still other embodiments, an “effective amount” refers to the amount of a therapeutic agent necessary to repair damaged neurons and/or induce regeneration of neurons.

As used herein, “active agent” refers to a physiologically or pharmacologically active substance that acts locally and/or systemically in the body. An active agent is a substance that is administered to a patient for the treatment (e.g., therapeutic agent), prevention (e.g., prophylactic agent), or diagnosis (e.g., diagnostic agent) of a disease or disorder.

As used herein, the term “naked” refers to binding proteins of the present disclosure that are not conjugated to another compound, e.g., a toxic compound or radiolabel. For example, the term “naked” can be used to refer to binding proteins such as di-scFv that are not conjugated to another compound. Accordingly, in some embodiments, binding proteins disclosed herein are “naked”. Put another way, the binding proteins of the present disclosure can be un-conjugated.

In contrast, the term “conjugated” is used in the context of the present disclosure to refer to binding proteins of the present disclosure that are conjugated to another compound, e.g., a toxic compound such as a cytotoxic agent or radiolabel. Accordingly, in some embodiments, the binding proteins of the present disclosure are “conjugated”.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other embodiments the values may range in value either above or below the stated value in a range of approx. +/−5%; in other embodiments the values may range in value either above or below the stated value in a range of approx. +/−2%; in other embodiments the values may range in value either above or below the stated value in a range of approx. +/−1%. The preceding ranges are intended to be made clear by context, and no further limitation is implied. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

### II. Compositions

Autoantibodies, such as those produced by Systemic Lupus Erythematosus (SLE) patients, are known to bind DNA and/or other nuclear components. Characterization of these anti-nuclear antibodies, as well as their derived single-chain variable fragments, has identified a subset that have cell-penetrating abilities and selective uptake in niches with high extracellular DNA, such as the tumor microenvironment (Weisbart, et al., Sci Rep. 2015 Jul. 9; 5:12022. doi: 10.1038/srep12022.). Several of these autoantibodies also have negative impacts on genomic integrity, either through inhibition of DNA repair (Hansen, et al., Sci Transl Med, 4 (157): 157ra142 (2012), Turchick, et al., Nucleic Acids Res. 2017 Nov. 16; 45 (20): 11782-11799, or through nucleolytic properties (Noble, et al., Noble, et al., Sci Rep-Uk, 4 (2014)). Both of these pathways can enhance the occurrence of ssDNA fragments (Wolf, et al., Nat Commun. 2016 May 27; 7:11752. doi: 10.1038/ncomms11752.) which can leak into the cytosol and activate innate immunity-mediated autoinflammation. In one pathway, the cGAS enzyme has been shown to be important for the antitumor effect of immune checkpoint inhibitors, and activation of the innate immunity pathway is synergistic with anti-PD-1/PD-L1 therapy.

It has been discovered that antibody 3E10 physically interacts with RAD51, defining a new molecular basis for HDR inhibition by 3E10 (Turchick, et al., Nucleic Acids Res. 2017 Nov. 16; 45 (20): 11782-11799, which is specifically incorporated by reference herein in its entirety). Utilizing purified 3E10 scFv protein and purified fragments of RAD51, 3E10 was shown to bind to the N-terminal domain of RAD51, a region important for homo-oligomerization and crucial for RAD51 filament formation, and can inhibit RAD51 accumulation on ssDNA and RAD51-dependent DNA strand exchange. Further, in keeping with this mechanism of action, 3E10 inhibits RAD51 foci formation in response to ionizing radiation or etoposide, a measurement of a cell's ability to form RAD51 nucleoprotein filaments at sites of DNA damage. Mutational analysis of the 3E10 variable region reveals separation-of-function linking RAD51 binding to inhibition of HDR and DNA binding to cell penetration.

The Examples below show that cells treated with the cell-penetrating antibody 3E10 also induce STAT1 activation (e.g., phosphorylated STAT1). Although phosphorylated STAT1 is a well-established marker of cGAS/STING inflammatory pathway activation, the results also indicate that this phosphorylation occurs in a cGAS independent manner in some cells.

Compositions and methods of using cell-penetrating antibodies in combination with an immune checkpoint modulator are provided.

Although the cell-penetrating molecules are generally referred to herein as “cell-penetrating binding proteins” or “cell-penetrating antibodies,” it will be appreciated that fragments, including antigen-binding fragments, variants, binding proteins and fusion proteins such as scFv, di-scFv, tri-scFv, and other single chain variable fragments, and other cell-penetrating molecules disclosed herein are also expressly provided for use in compositions and methods disclosed herein. The methods typically include administering to a subject in need thereof an effective amount of an immune checkpoint modulator and a cell-penetrating binding protein, such as an antibody, that (1) induces DNA damage, (2) impairs DNA damage repair, or (3) a combination thereof. The immune checkpoint modulator and the cell-penetrating binding protein, such as an antibody, can be administered to the subject together or separately.

Compositions include an effective amount of the immune checkpoint modulator and cell-penetrating binding protein, such as an antibody, are also provided.

A. Cell-penetrating anti-DNA Binding Proteins

The disclosed methods typically include administering a subject an effective amount of a cell-penetrating anti-DNA binding protein such as an antibody that induces DNA damage, reduces or impairs DNA damage repair, or a combination thereof. In some embodiments, the cell-penetrating anti-DNA binding protein impairs DNA damage repair. Examples of anti-DNA binding proteins that impair DNA repair are discussed below. In some embodiments, the cell-penetrating anti-DNA binding protein is administered in an effective amount to induce the formation or increase the presence of ssDNA fragments in the cytosol of cells, for example cancer or infected cells, of the subject. In some embodiments, the anti-DNA binding protein is administered in an effective amount to induce the cGAS/STING inflammatory pathway. In some embodiments, the anti-DNA binding protein is administered in an effective amount to induce STAT1 activation (e.g., STAT1 phosphorylation), or another marker or indicator of GAS/STING activation. In some embodiments, STAT1 phosphorylation and/or the level of phosphorylated STAT1 in the cells is cGAS independent.

Cytosolic DNA, either endogenous self-DNA or DNA from pathogens, can activate the cGAS/STING pathway. Briefly, cytosolic DNA activates cGAS which leads to the production of cyclic GMP-AMP (cGAMP) from cellular ATP and GTP. cGAMP then acts as a ligand for the STING protein. The STING protein recruits and activates TBK1. Activated TBK1 then phosphorylates IRF3, which leads to the dimerization of phospho-IRF3. The IRF3 dimer then translocates to the nucleus and acts as a transcription factor to induce the expression of type I interferons and inflammatory cytokines. Autocrine signaling by the type I interferons leads to cytoplasmic activation of STAT1 and STAT2, which translocate to the nucleus to induce the expression of interferon-stimulated genes (Chen, et al., Nat Immunol (2016) 17 (10): 1142-9.10.1038/ni.3558).

Multiple steps in the cGAS/STING pathway can be monitored to track activation of the innate immune response using methods known in the art. For example, phosphorylation of TBK1, IRF3, STAT1 and STAT2 can be monitored by western blot or immunofluorescence. cGAS stimulation can also be monitored by western blot or immunofluorescence. For example, excessive DNA damage that accumulates in cycling cells is sequestered into micronuclei; these micronuclei are often cGAS positive and can be identified by microscopy and immunofluorescence. Induction of expression of type I interferons and inflammatory cytokines can be monitored by RT-PCR and western blot.

Cell-penetrating antibodies and binding proteins that can induce DNA damage and/or reduce or impair DNA damage repair are known in the art. For example, select lupus anti-DNA autoantibodies can penetrate into live cell nuclei and impair DNA repair or directly damage DNA, and efforts to use these antibodies against tumors that are sensitive to DNA damage are underway (Hansen, et al., Sci Transl Med, 4 (157): 157ra142 (2012), Noble, et al., Cancer Research, 2015; 75 (11): 2285-2291, Noble, et al., Sci Rep-Uk, 4 (2014), Noble, et al., Nat Rev Rheumatol (2016)). Therefore, in some embodiments, anti-DNA antibodies can be derived or isolated from patients with SLE. In some embodiments, the anti-DNA antibodies are monoclonal antibodies, or fragments or variants thereof.

Exemplary antibodies that can be used include whole immunoglobulin (i.e., an intact antibody) of any class, fragments thereof, and synthetic proteins containing at least the antigen binding variable domain of an antibody. The variable domains differ in sequence among antibodies and are used in the binding and specificity of each particular antibody for its particular antigen. However, the variability is not usually evenly distributed through the variable domains of antibodies. It is typically concentrated in three segments called complementarity determining regions (CDRs) or hypervariable regions both in the light chain and the heavy chain variable domains. The more highly conserved portions of the variable domains are called the framework (FR). The variable domains of native heavy and light chains each include four FR regions, largely adopting a beta-sheet configuration, connected by three CDRs, which form loops connecting, and in some cases forming part of, the beta-sheet structure. The CDRs in each chain are held together in close proximity by the FR regions and, with the CDRs from the other chain, contribute to the formation of the antigen binding site of antibodies. Therefore, the antibodies can contain the components of the CDRs necessary to penetrate cells, maintain DNA binding and/or interfere with DNA repair.

Also disclosed are variants and fragments of antibodies which have bioactivity. The fragments, whether attached to other sequences or not, include insertions, deletions, substitutions, or other selected modifications of particular regions or specific amino acids residues, provided the activity of the fragment is not significantly altered or impaired compared to the non-modified antibody or antibody fragment.

Techniques can also be adapted for the production of single-chain antibodies specific to an antigenic protein. Methods for the production of single-chain antibodies are well known to those of skill in the art. A single chain antibody can be created by fusing together the variable domains of the heavy and light chains using a short peptide linker, thereby reconstituting an antigen binding site on a single molecule. Single-chain antibody variable fragments (scFvs) in which the C-terminus of one variable domain is tethered to the N-terminus of the other variable domain via a 15 to 25 amino acid peptide or linker have been developed without significantly disrupting antigen binding or specificity of the binding. The linker is chosen to permit the heavy chain and light chain to bind together in their proper conformational orientation.

The anti-DNA antibodies can be modified to improve their therapeutic potential. For example, in some embodiments, the anti-DNA antibody is conjugated to another antibody specific for a second therapeutic target, for example, on or near a cancer cell or in a tumor microenvironment. For example, the anti-DNA antibody can be a fusion protein containing single chain variable fragment that binds DNA or nucleosomes and a single chain variable fragment of a monoclonal antibody that specifically binds the second therapeutic target. In other embodiments, the anti-DNA antibody is a bispecific antibody having a first heavy chain and a first light chain from an anti-DNA antibody and a second heavy chain and a second light chain from a monoclonal antibody that specifically binds the second therapeutic target.

Divalent single-chain variable fragments (di-scFvs) can be engineered by linking two scFvs. This can be done by producing a single peptide chain with two V_{H} and two V_{L} regions, yielding tandem scFvs. ScFvs can also be designed with linker peptides that are too short for the two variable regions to fold together (about five amino acids), forcing scFvs to dimerize. This type is known as diabodies. Diabodies have been shown to have dissociation constants up to 40-fold lower than corresponding scFvs, meaning that they have a much higher affinity to their target. Still shorter linkers (one or two amino acids) lead to the formation of trimers (triabodies or tribodies). Tetrabodies have also been produced. They exhibit an even higher affinity to their targets than diabodies.

The antibody can be a humanized or chimeric antibody, or a fragment, variant, or fusion protein thereof. Methods for humanizing non-human antibodies are well known in the art. Generally, a humanized antibody has one or more amino acid residues introduced into it from a source that is non-human. These non-human amino acid residues are often referred to as “import” residues, which are typically taken from an “import” variable domain. Antibody humanization techniques generally involve the use of recombinant DNA technology to manipulate the DNA sequence encoding one or more polypeptide chains of an antibody molecule.

In some embodiments, the antibody is modified to alter its half-life. In some embodiments, it is desirable to increase the half-life of the antibody so that it is present in the circulation or at the site of treatment for longer periods of time. In other embodiments, the half-life of the anti-DNA antibody is decreased to reduce potential side effects. Antibody fragments are expected to have a shorter half-life than full size antibodies. Other methods of altering half-life are known and can be used in the described methods. For example, antibodies can be engineered with Fc variants that extend half-life, e.g., using Xtend™ antibody half-life prolongation technology (Xencor, Monrovia, CA).

In some embodiments, the antibody is conjugated to a cell-penetrating moiety, such as a cell-penetrating peptide, to facilitate entry into the cell and transport to the nucleus. Examples of cell-penetrating peptides include, but are not limited to, Polyarginine (e.g., R9), Antennapedia sequences, TAT, HIV-Tat, Penetratin, Antp-3A (Antp mutant), Buforin II, Transportan, MAP (model amphipathic peptide), K-FGF, Ku70, Prion, pVEC, Pep-1, SynB1, Pep-7, HN-1, BGSC (Bis-Guanidinium-Spermidine-Cholesterol, and BGTC (Bis-Guanidinium-Tren-Cholesterol). In other embodiments, the antibody is modified using TransMabs™ technology (InNexus Biotech., Inc., Vancouver, BC).

In some embodiments, the anti-DNA antibody is 3E10, 5C6, or a variant, functional fragment, or fusion protein derived therefrom. For example, the anti-DNA antibody can have a V_{H} having an amino acid sequence as shown in SEQ ID NO:6 or 7 and a V_{L} having an amino acid sequence as shown in SEQ ID NO:1 and 2 (3E10). Exemplary variants include antibodies having a V_{H} including an amino acid sequence at least 90% identical to the amino acid sequence shown in SEQ ID NO:6 or 7 and a V_{L} including an amino acid sequence at least 90% identical to the sequence as shown in SEQ ID NO:1 or 2. Other exemplary variants include antibodies having a V_{H} including an amino acid sequence at least 95%, at least 98%, at least 99% identical to the amino acid sequence shown in SEQ ID NO:6 or 7 and a V_{L} including an amino acid sequence at least 95%, at least 98%, at least 99% identical to the sequence as shown in SEQ ID NO:1 or 2. Other exemplary variants include humanized forms of 3E10 such as those described in WO 2015/106290 and WO 2016/033324, and those provided below.

In another example, the anti-DNA antibody can have a V_{H} having an amino acid sequence as shown in SEQ ID NO:16 and a V_{L} having an amino acid sequence as shown in SEQ ID NO:12 (5C6). Exemplary variants include antibodies having a V_{H} having an amino acid sequence at least 90% identical to the amino acid sequence shown in SEQ ID NO:16 and a V_{L} having an amino acid sequence at least 90% identical to the sequence as shown in SEQ ID NO: 12. Other exemplary variants include antibodies having a V_{H} having an amino acid sequence at least 95%, at least 98%, at least 99% identical to the amino acid sequence shown in SEQ ID NO:16 and a V_{L} having an amino acid sequence at least 95%, at least 98%, at least 99% identical to the sequence as shown in SEQ ID NO:12.

### 1. Exemplary Binding Proteins

A panel of hybridomas, including the 3E10 and 5C6 hybridomas was previously generated from the MRLmpj/lpr lupus mouse model and DNA binding activity was evaluated (Zack, et al., J. Immunol. 154:1987-1994 (1995); Gu, et al., J. Immunol., 161:6999-7006 (1998)). Murine 3E10 can refer to the monoclonal antibody produced by ATCC Accession No. PTA 2439 hybridoma. 5C6 can refer to the monoclonal anti-DNA antibody with nucleolytic activity produced by a hybridoma from MRL/lpr lupus mouse model as described in Noble et al., 2014, Sci Rep 4:5958 doi: 10.1038/srep05958.

Thus in some embodiments, the cell-penetrating antibody is 3E10 or 5C6 antibody or a variant, fragment, and fusion protein thereof, or a humanized form thereof. Each can be used, alone or in combination, in the disclosed methods.

### a. 3E10

In the early 1990s a murine lupus anti-DNA antibody, 3E10, was tested in experimental vaccine therapy for SLE. These efforts were aimed at developing anti-idiotype antibodies that would specifically bind anti-DNA antibody in SLE patients. However, 3E10 was serendipitously found to penetrate into living cells and nuclei without causing any observed cytotoxicity (Weisbart R H, et al. J Immunol. 1990 144 (7): 2653-2658; Zack D J, et al. J Immunol. 1996 157 (5): 2082-2088). Studies on 3E10 in SLE vaccine therapy were then supplanted by efforts focused on development of 3E10 as a molecular delivery vehicle for transport of therapeutic molecules into cells and nuclei. 3E10 preferentially binds DNA single-strand tails, inhibits key steps in DNA single-strand and double-strand break repair (Hansen, et al., Science Translational Medicine, 4: 157ra142 (2012)). The 3E10 antibody and its single chain variable fragment which includes a D31N mutation in CDR1 of the V_{H} (3E10 (D31N) scFv) and di- and tri-valent fusions thereof penetrate into cells and nuclei and have proven capable of transporting therapeutic protein cargoes attached to the antibody either through chemical conjugation or recombinant fusion. Protein cargoes delivered to cells by 3E10 or 3E10 (D31N) scFv include catalase, p53, and Hsp70 (Weisbart R H, et al. J Immunol. 2000 164:6020-6026; Hansen J E, et al. Cancer Res. 2007 Feb. 15; 67 (4): 1769-74; Hansen J E, et al. Brain Res. 2006 May 9; 1088 (1): 187-96). 3E10 (D31N) scFv effectively mediated delivery of Hsp70 to neurons in vivo and this resulted in decreased cerebral infarct volumes and improved neurologic function in a rat stroke model (Zhan X, et al. Stroke. 2010 41 (3): 538-43).

3E10 and 3E10 (D31N) scFv and di- and tri-valent fusions thereof, without being conjugated to any therapeutic protein, enhance cancer cell radiosensitivity and chemosensitivity and that this effect is potentiated in cells deficient in DNA repair.

Moreover, 3E10 and 3E10 scFv and di- and tri-valent fusions thereof are selectively lethal to cancer cells deficient in DNA repair even in the absence of radiation or chemotherapy. The Food and Drug Administration (FDA) has established a pathway for the development of monoclonal antibodies into human therapies, and 3E10 has already been approved by the FDA for use in a Phase I human clinical trial designed to test the efficacy of 3E10 in experimental vaccine therapy for SLE (Spertini F, et al. J Rheumatol. 1999 26 (12): 2602-8). Experiments indicate that 3E10 (D31N) scFv penetrates cell nuclei by first binding to extracellular DNA or its degradation products and then following them into cell nuclei through the ENT2 nucleoside salvage pathway (Weisbart, Scientific Reports, 5: Article number: 12022 (2015) doi: 10.1038/srep12022). When administered to mice and rats 3E10 is preferentially attracted to tissues in which extracellular DNA is enriched, including tumors, regions of ischemic brain in stroke models, and skeletal muscle subject to contractile injury (Weisbart, et al., Sci Rep., 5:12022 (2015), Hansen, et al., J Biol Chem, 282 (29): 20790-20793 (2007), Weisbart, et al., Mol Immunol, 39 (13): 783-789 (2003), Zhan, et al., Stroke: A Journal of Cerebral Circulation, 41 (3): 538-543 (2010)). Thus the presence of extracellular DNA enhances the nuclear uptake of 3E10 (D31N) scFv. Furthermore, 3E10 (D31N) scFv preferentially localizes into tumor cell nuclei in vivo, likely due to increased DNA in the local environment released from ischemic and necrotic regions of tumor.

### b. 5C6

5C6 induces γH2AX in BRCA2⁽⁻⁾ but not BRCA2⁽⁺⁾ cells and selectively suppresses the growth of the BRCA2° cells. Mechanistically, 5C6 appears to induce senescence in the BRCA2⁽⁻⁾ cells. Senescence is a well-known response to DNA damage, and DNA damaging agents, including many chemotherapeutics, induce senescence after prolonged exposure (Sliwinska, et al., Mech. Ageing Dev., 130:24-32 (2009); the Poele, et al., Cancer Res. 62:1876-1883 (2002); Achuthan, et al., J. Biol. Chem., 286:37813-37829 (2011)). These observations establish that 5C6 penetrates cell nuclei and damages DNA, and that cells with preexisting defects in DNA repair due to BRCA2 deficiency are more sensitive to this damage than cells with intact DNA repair. See U.S. Published Application No. 2015/0376279.

### 2. Fragments and Fusion Proteins

In some embodiments, the antibody is one or more antigen binding antibody fragments and/or antigen binding fusion proteins of the antibody 3E10 or 5C6, or a variant thereof. The antigen binding molecules typically bind to the epitope of 3E10 or 5C6, and can, for example, maintain a function or activity of the full antibody.

Exemplary fragments and fusions include, but are not limited to, single chain antibodies, single chain variable fragments (scFv), di-scFv, tri-scFv, diabody, triabody, tetrabody, disulfide-linked Fvs (sdFv), Fab′, F(ab′)₂, Fv, and single domain antibody fragments (sdAb).

In some embodiments, the antibody includes two or more scFv. For example, the targeting moiety can be a scFv or a di-scFv. In some embodiments, each scFv can include one, two, or all three complementarity determining regions (CDRs) of the heavy chain variable region (V_{L}) of 3E10 or 5C6, or a variant thereof. The scFv can include one, two, or all three CDRs of the light chain variable region (V_{L}) of 3E10 or 5C6, or a variant thereof. The molecule can include the heavy chain variable region and/or light chain variable region of 3E10 or 5C6, or a variant thereof.

A single chain variable fragment can be created by fusing together the variable domains of the heavy and light chains using a short peptide linker, thereby reconstituting an antigen binding site on a single molecule. Single-chain antibody variable fragments (scFvs) in which the C-terminus of one variable domain is tethered to the N-terminus of the other variable domain via a linker have been developed without significantly disrupting antigen binding or specificity of the binding. The linker is chosen to permit the heavy chain and light chain to bind together in their proper conformational orientation. The linker is usually rich in glycine for flexibility, and typically also includes serine or threonine for solubility. The linker can link, for example, the N-terminus of the V_{H} with the C-terminus of the V_{L}, or vice versa. scFv can also be created directly from subcloned heavy and light chains derived from a hybridoma. In some embodiments, the scFv retains, or improves or increases the specificity of the original immunoglobulin, while removing of the constant regions and introducing the linker.

Exemplary molecules that include two or more single chain variable fragments (scFv) including the light chain variable region (V_{L}) of 3E10 or 5C6, or a variant thereof, and the heavy chain variable region (V_{H}) of 3E10 or 5C6, or a variant thereof of the antibody 3E10 or 5C6 include, but are not limited to, divalent-scFv (di-scFv), trivalent-scFv (tri-scFv), multivalent-scFv (multi-scFv), diabodies, triabodies, tetrabodies, etc., of scFvs. Divalent single chain variable fragments can be engineered by linking two scFvs. This can be done by producing a single peptide chain with two V_{H} and two V_{L} regions, yielding a di-scFvs referred to as a tandem di-scFv. ScFvs can also be designed with linker peptides that are too short for the two variable regions to fold together (about five amino acids), forcing scFvs to dimerize and form a divalent single chain variable fragment referred to as a diabody. Diabodies have been shown to have dissociation constants up to 40-fold lower than corresponding scFvs, indicating that they have a much higher affinity to their target. Even shorter linkers (one or two amino acids) lead to the formation of trimers (triabodies or tribodies). Tetrabodies have also been produced and have been shown to exhibit an even higher affinity to their targets than diabodies.

The disclosed antibodies include antigen binding antibody fragments and fusion proteins of 3E10 or 5C6 and variants thereof that can bind to the same epitope as the parent antibody 3E10 or 5C6. In some embodiments, the antigen binding molecule is a di-, tri-, or multivalent scFv. Although the antigen binding antibody fragment or fusion protein of the antigen binding molecule can include additional antibody domains (e.g., constant domains, hinge domains, etc.,), in some embodiments it does not. For example, 3E10 binds DNA and impairs DNA repair, which is synthetically lethal to DNA repair-deficient cells. This function is independent of any 3E10 constant regions. By contrast, non-penetrating antibodies such as cetuximab that target extracellular receptors depend in part on Fc-mediated activation of ADCC and complement to exert an effect on tumors. Elimination of the Fc from non-penetrating antibodies could therefore diminish the magnitude of their effect on tumors, but Fc is not required for 3E10 to have an effect on cancer cells. Therefore, 3E10 fragments or fusions that lack an Fc region should be unable to activate ADCC and complement and therefore carry a lower risk of nonspecific side effects.

a. Single Chain Variable Fragments

The single chain variable fragments disclosed herein can include antigen binding fragments of 3E10 or 5C6, or a variant thereof. The monoclonal antibody 3E10 and active fragments and exemplary variants thereof that are transported in vivo to the nucleus of mammalian cells without cytotoxic effect are discussed in U.S. Pat. Nos. 4,812,397 and 7,189,396, and U.S. Published Application No. 2014/0050723. Other 3E10 antibody compositions, including fragments and fusions thereof, suitable for use with the disclosed compositions and methods are discussed in, for example, WO 2012/135831, WO 2016/033321, WO 2015/106290, and WO 2016/033324. 5C6 is described in U.S. Published Application No. 2015/0376279. Sequences for single and two or more linked single chain variable fragments of 3E10 are provided in WO 2017/218825 and WO 2016/033321. Exemplary 3E10 humanized sequences are discussed in WO 2015/106290 and WO 2016/033324.

An scFv includes a light chain variable region (V_{L}) and a heavy chain variable region (V_{H}) joined by a linker. For example, the linker can include in excess of 12 amino acid residues with (Gly4Ser) 3 (SEQ ID NO:26) being one of the more favored linkers for a scFv. The scFv can be a disulfide stabilized Fv (or diFv or dsFv), in which a single cysteine residue is introduced into a FR of V_{H} and a FR of V_{L} and the cysteine residues linked by a disulfide bond to yield a stable Fv. The scFv can be a dimeric scFv (di-scFV), i.e., a protein including two scFv molecules linked by a non-covalent or covalent linkage, e.g., by a leucine zipper domain (e.g., derived from Fos or Jun) or trimeric scFV (tri-scFv). In another example, two scFv's are linked by a peptide linker of sufficient length to permit both scFv's to form and to bind to an antigen, e.g., as described in U.S. Published Application No. 2006/0263367.

The variable domains differ in sequence among antibodies and are used in the binding and specificity of each particular antibody for its particular antigen. However, the variability is not usually evenly distributed through the variable domains of antibodies. It is typically concentrated in three segments called complementarity determining regions (CDRs) or hypervariable regions both in the light chain and the heavy chain variable domains. The more highly conserved portions of the variable domains are called the framework (FR). The variable domains of native heavy and light chains each include four FR regions, largely adopting a beta-sheet configuration, connected by three CDRs, which form loops connecting, and in some cases forming part of, the beta-sheet structure. The CDRs in each chain are held together in close proximity by the FR regions and, with the CDRs from the other chain, contribute to the formation of the antigen binding site of antibodies.

The fragments and fusions of antibodies disclosed herein can have bioactivity. For example, the fragments and fusions, whether attached to other sequences or not, can include insertions, deletions, substitutions, or other selected modifications of particular regions or specific amino acids residues. In some embodiments, the activity of the fragment or fusion is not significantly reduced or impaired compared to the nonmodified antibody or antibody fragment.

b. Sequences

### i. 3E10 Light Chain Variable Region

An amino acid sequence for the light chain variable region of 3E10 is:

(SEQ ID NO: 1)
DIVLTQSPASLAVSLGQRATISCRASKSVSTSSYSYMHWYQQKPGQPPKL
LIKYASYLESGVPARFSGSGSGTDFTLNIHPVEEEDAATYYCQHSREFPW
TFGGGTKLEIK.

The complementarity determining regions (CDRs) as defined by Kabat are shown with underlining. Other 3E10 light chain sequences are known in the art. See, for example, Zack, et al., J. Immunol., 15;154 (4): 1987-94 (1995); GenBank: L16981.1-Mouse Ig rearranged L-chain gene, partial cds; GenBank: AAA65681.1-immunoglobulin light chain, partial [Mus musculus]).

An amino acid sequence for the light chain variable region of 3E10 can also be:

(SEQ ID NO: 2)
DIVLTQSPASLAVSLGQRATISCRASKSVSTSSYSYMHWYQQKPGQPPKL
LIKYASYLESGVPARFSGSGSGTDFHLNIHPVEEEDAATYYCQHSREFPW
TFGGGTKLELK.

The complementarity determining regions (CDRs) as defined by Kabat are shown with underlining, including CDR L1.1:

Variants of Kabat CDR L1.1 include

CDR L1.1:
(SEQ ID NO: 34)
RASKSVSTSSYSYMH;
CDR L2.1:
(SEQ ID NO: 36)
YASYLES;
CDR L3.1:
(SEQ ID NO: 37)
QHSREFPWT.
(SEQ ID NO: 91)
RASKSVSTSSYSYLA
and
(SEQ ID NO: 35)
RASKTVSTSSYSYMH.
(SEQ ID NO: 90)
YASYLQS.

A variant of Kabat CDR L2.1 is

Additionally, or alternatively, the heavy chain complementarity determining regions (CDRs) can be defined according to the IMGT system. The complementarity determining regions (CDRs) as defined by the IMGT system include CDR L1.2 KSVSTSSYSY (SEQ ID NO: 42); CDR L2.2: YAS (SEQ ID NO:44); CDR L3.2: QHSREFPWT (SEQ ID NO:37).

A variant of CDR L1.2 is KTVSTSSYSY (SEQ ID NO:43).

In some embodiments, the C-terminal end of sequence of SEQ ID NOS: 1 or 2 further includes an arginine in the 3E10 light chain variable region.

### ii. 3E10 Heavy Chain Variable Region

An amino acid sequence for the heavy chain variable region of 3E10 is:

(SEQ ID NO: 6
EVQLVESGGGLVKPGGSRKLSCAASGFTFSDYGMHWVRQAPEKGLEWVAY
ISSGSSTIYYADTVKGRFTISRDNAKNTLFLQMTSLRSEDTAMYYCARRG
LLLDYWGQGTTLTVSS;

Zack, et al., Immunology and Cell Biology, 72:513-520 (1994); GenBank: L16981.1-Mouse Ig rearranged L-chain gene, partial cds; and GenBank: AAA65679.1-immunoglobulin heavy chain, partial [Mus musculus]). The complementarity determining regions (CDRs) as defined by Kabat are shown with underlining.

An amino acid sequence for a preferred variant of the heavy chain variable region of 3E10 is:

(SEQ ID NO: 7)
EVQLVESGGGLVKPGGSRKLSCAASGFTFSNYGMHWVRQAPEKGLEWVAY
ISSGSSTIYYADTVKGRFTISRDNAKNTLFLQMTSLRSEDTAMYYCARRG
LLLDYWGQGTTLTVSS.

The complementarity determining regions (CDRs) as defined by Kabat are shown with underlining.

In some embodiments, the C-terminal serine of SEQ ID NOS: 6 or 7 is absent or substituted, with, for example, an alanine, in 3E10 heavy chain variable region.

Amino acid position 31 of the heavy chain variable region of 3E10 has been determined to be influential in the ability of the antibody and fragments thereof to penetrate nuclei and bind to DNA. For example, D31N mutation (bolded and italicized in SEQ ID NOS: 1 and 2) in CDR1 penetrates nuclei and binds DNA with much greater efficiency than the original antibody (Zack, et al., Immunology and Cell Biology, 72:513-520 (1994), Weisbart, et al., J. Autoimmun., 11, 539-546 (1998); Weisbart, Int. J. Oncol., 25, 1867-1873 (2004)).

The complementarity determining regions (CDRs) as defined by Kabat are shown with underlining, including CDR H1.1 (original sequence): DYGMH (SEQ ID NO:8); CDR H1.2 (with D31N mutation): NYGMH (SEQ ID NO:30); CDR H2.1: YISSGSSTIYYADTVKG (SEQ ID NO:10); CDR H3.1: RGLLLDY (SEQ ID NO:33).

Variants of Kabat CDR H2.1 include YISSGSSTIYYADSVKG (SEQ ID NO:32) and YISSSSSTIYYADSVKG (SEQ ID NO:31).

Additionally, or alternatively, the heavy chain complementarity determining regions (CDRs) can be defined according to the IMGT system. The complementarity determining regions (CDRs) as defined by the IMGT system include CDR H1.3 (original sequence): GFTFSDYG (SEQ ID NO:89); CDR H1.4 (with D31N mutation): GFTFSNYG (SEQ ID NO: 38); CDR H2.2: ISSGSSTI (SEQ ID NO:40); CDR H3.2: ARRGLLLDY (SEQ ID NO: 41).

A variant of CDR H2.2 is ISSSSSTI (SEQ ID NO:39).

In addition to 3E10 and its fragments described above, additional anti-DNA antibodies may be used in the disclosed compositions and methods. These include the nuclear-penetrating anti-DNA antibody 5C6 as specified below.

iii. 5C6 Light Chain Variable Region

An amino acid sequence for the kappa light chain variable region (VL) of mAb 5C6 is:

(SEQ ID NO: 12)
DIVLTQSPASLAAVSLGERATISYRASKSVSTSGYSYMHWNQQKPGQAPR
LLIYLVSNLESGVPARFSGSGSGTDFTLNIHPVEEEDAATYYCQHIRELD
TFFGGGTKLEIK.

The complementarity determining regions (CDRs) are shown with underlining, including CDR L1: RASKSVSTSGYSYMH (SEQ ID NO:13); CDR L2: LVSNLES (SEQ ID NO: 14); CDR L3: QHIRELDTF (SEQ ID NO:15).

### iv. 5C6 Heavy Chain Variable Region

An amino acid sequence for the heavy chain variable region (VH) of mAb 5C6 is:

(SEQ ID NO: 16)
QLKLVESGGGLVKPGGSLKLSCAASGFTFSSYTMSWVRQTPAKRLEWVAT
ISSGGGSTYYPDSVKGRFTISRDNARNTLYLQMSSLRSEDTAMYYCARRA
YSKRGAMDYWGQGTSVTVSS.

The complementarity determining regions (CDRs) are shown with underlining, including CDR H1: SYTMS (SEQ ID NO:17); CDR H2: TISSGGGSTYYPDSVKG (SEQ ID NO: 18); CDR H3: RAYSKRGAMDY (SEQ ID NO:19).

c. Linkers

The term “linker” as used herein includes, without limitation, peptide linkers. The peptide linker can be any size provided it does not interfere with the binding of the epitope by the variable regions. In some embodiments, the linker includes one or more glycine and/or serine amino acid residues. Monovalent single-chain antibody variable fragments (scFvs) in which the C-terminus of one variable domain are typically tethered to the N-terminus of the other variable domain via a 15 to 25 amino acid peptide or linker. The linker is chosen to permit the heavy chain and light chain to bind together in their proper conformational orientation. Linkers in diabodies, triabodies, etc., typically include a shorter linker than that of a monovalent scFv as discussed above. Di-, tri-, and other multivalent scFvs typically include three or more linkers. The linkers can be the same, or different, in length and/or amino acid composition. Therefore, the number of linkers, composition of the linker(s), and length of the linker(s) can be determined based on the desired valency of the scFv as is known in the art. The linker(s) can allow for or drive formation of a di-, tri-, and other multivalent scFv.

For example, a linker can include 4-8 amino acids. In a particular embodiment, a linker includes the amino acid sequence GQSSRSS (SEQ ID NO:20). In another embodiment, a linker includes 15-20 amino acids, for example, 18 amino acids. In a particular embodiment, the linker includes the amino acid sequence GQSSRSSSGGGSSGGGGS (SEQ ID NO:21). Other flexible linkers include, but are not limited to, the amino acid sequences Gly-Ser, Gly-Ser-Gly-Ser (SEQ ID NO:22), Ala-Ser, Gly-Gly-Gly-Ser (SEQ ID NO:23), (Gly4-Ser)₂ (SEQ ID NO:24) and (Gly4-Ser) 4 (SEQ ID NO: 25), and (Gly-Gly-Gly-Gly-Ser) 3 (SEQ ID NO:26).

d. Variants

The antibody can be composed of or include an antibody fragment or fusion protein including an amino acid sequence of a variable heavy chain and/or variable light chain that is at least 45%, at least 50%, at least 55%, at least 60%, at least 65%, at least 70%, at least 75%, at least 80%, at least 85%, at least 90%, at least 95%, or at least 99% identical to the amino acid sequence of the variable heavy chain and/or light chain of 3E10 or 5C6 or a humanized form thereof, including to any of the exemplary sequences provided herein. In some embodiments, the antibody binds to the epitope of 3E10 or 5C6, is selectively lethal to or selectively increases the radiosensitivity and/or chemosensitivity of cells deficient in DNA repair, or a combination thereof.

The antibody can be composed of or include an antibody fragment or fusion protein that includes a CDR that is at least 45%, at least 50%, at least 55%, at least 60%, at least 65%, at least 70%, at least 75%, at least 80%, at least 85%, at least 90%, at least 95%, or at least 99% identical to the amino acid sequence of a CDR of the variable heavy chain and/or light chain of 3E10 or 5C6 and/or a humanized form thereof, including to any of the exemplary sequences provided herein. In some embodiments, the antibody binds to the epitope of 3E10 or 5C6, is selectively lethal to or selectively increases the radiosensitivity and/or chemosensitivity of cells deficient in DNA repair, or a combination thereof.

The determination of percent identity of two amino acid sequences can be determined by BLAST protein comparison. In some embodiments, scFv includes one, two, three, four, five, or all six of the CDRs of the above-described preferred variable domains and which binds to the epitope of 3E10 or 5C6, is selectively lethal to or selectively increases the radiosensitivity and/or chemosensitivity of cells deficient in DNA repair, or a combination thereof.

Predicted complementarity determining regions (CDRs) of the light chain variable sequence for 3E10 or 5C6 are provided above. See also GenBank: AAA65681.1-immunoglobulin light chain, partial [Mus musculus]. Predicted complementarity determining regions (CDRs) of the heavy chain variable sequence for 3E10 and 5C6 are provide above. See, for example, Zack, et al., Immunology and Cell Biology, 72:513-520 (1994) and GenBank Accession number AAA65679.1. Exemplary humanized 3E10 sequences and scFv are provided below.

### e. Exemplary Humanized anti-DNA Binding Proteins

Exemplary anti-DNA binding proteins, and exemplary human IgG1 hinge and constant regions are disclosed in International Patent Application PCT/US2018/042532, and International Patent Application PCT/US2018/042534, and provided below. Cell-penetrating antibodies for use in the disclosed combination therapies include those having the exemplary humanized CDR, the exemplary humanized heavy chain variable regions, and/or the exemplary humanized light chain variable regions, and fragments and variants thereof.

The binding proteins and antibodies herein can have, for example, any combination of light and heavy chain CDR1-3 sequences provided herein. The binding protein and antibodies herein can have, for example any combination of light and heavy chain region sequences provided herein.

In some embodiments, the anti-DNA binding proteins include a V_{H} having a CDR1 as shown in SEQ ID NO:30, a CDR2 as shown in SEQ ID NO:31 or SEQ ID NO:32 and a CDR3 as shown in SEQ ID NO:33 and a V_{L} having a CDR1 as shown in SEQ ID NO:34 or SEQ ID NO:35, a CDR2 as shown in SEQ ID NO:36 and a CDR3 as shown in SEQ ID NO: 37. For example, an anti-DNA binding protein can include a V_{H} having a CDR1 as shown in SEQ ID NO:30, a CDR2 as shown in SEQ ID NO:31 and a CDR3 as shown in SEQ ID NO: 33 and a V_{L} having a CDR1 as shown in SEQ ID NO:34, a CDR2 as shown in SEQ ID NO: 36 and a CDR3 as shown in SEQ ID NO:37. In another embodiment, an anti-DNA binding protein can include a V_{H} having a CDR1 as shown in SEQ ID NO:30, a CDR2 as shown in SEQ ID NO:31 and a CDR3 as shown in SEQ ID NO:33 and a V_{L} having a CDR1 as shown in SEQ ID NO:35, a CDR2 as shown in SEQ ID NO:36 and a CDR3 as shown in SEQ ID NO:37. In another embodiment, an anti-DNA binding protein can include a V_{H} having a CDR1 as shown in SEQ ID NO:30, a CDR2 as shown in SEQ ID NO:32 and a CDR3 as shown in SEQ ID NO:33 and a V_{L} having a CDR1 as shown in SEQ ID NO:34, a CDR2 as shown in SEQ ID NO:36 and a CDR3 as shown in SEQ ID NO:37. In another embodiment, an anti-DNA binding protein can include a V_{H} having a CDR1 as shown in SEQ ID NO: 30, a CDR2 as shown in SEQ ID NO:32 and a CDR3 as shown in SEQ ID NO:33 and a V_{L} having a CDR1 as shown in SEQ ID NO:35, a CDR2 as shown in SEQ ID NO:36 and a CDR3 as shown in SEQ ID NO:37.

Above exemplified binding proteins may also have CDRs assigned using the IMGT system. Appropriate sequences from this system are referenced below.

In another embodiment, the anti-DNA binding proteins include a V_{H} including a sequence at least 95% identical to the sequence as shown in any one of SEQ ID NOs: 9, 11, or 45 to 52 and a V_{L} including a sequence at least 95% identical to the sequence as shown in any one of SEQ ID NOs: 3 to 5, or 53 to 58. For example, an anti-DNA binding protein can include a V_{H} including a sequence at least 95% identical to the sequence as shown in SEQ ID NO: 47 and a V_{L} including a sequence at least 95% identical to the sequence as shown in SEQ ID NO: 54. In another embodiment, an anti-DNA binding protein can include a V_{H} including a sequence at least 95% identical to the sequence as shown in SEQ ID NO:52 and a V_{L} including a sequence at least 95% identical to the sequence as shown in SEQ ID NO:56. In these embodiments, the V_{H} and/or V_{L} can be at least 96%, at least 97%, at least 98% or at least 99% identical to the recited SEQ ID NO.

In some embodiments, the anti-DNA binding proteins include a V_{H} including a sequence as shown in any one of SEQ ID NOs: 9, 11, or 45 to 52 and a V_{L} including a sequence as shown in any one of SEQ ID NOs: 3 to 5 or 53 to 58. For example, an anti-DNA binding protein can include a V_{H} including a sequence as shown in SEQ ID NO:47 and a V_{L} including a sequence as shown in SEQ ID NO:54. In another embodiment, an anti-DNA binding protein can include a V_{H} including a sequence as shown in SEQ ID NO:52 and a V_{L} including a sequence as shown in SEQ ID NO:56.

In some embodiments, the anti-DNA binding protein can be a cell-penetrating anti-DNA Fv fragment having an antigen binding domain, wherein the antigen binding domain binds to or specifically binds to DNA. For example, the Fv can bind the same epitope as a binding protein having a V_{H} including an amino acid sequence as shown in SEQ ID NO:7 and a V_{L} including an amino acid sequence as shown in SEQ ID NO:2. In another embodiment, the Fv can bind the same epitope as a di-scFv having an amino acid sequence as shown in SEQ ID NO:28. In some embodiments, the Fv includes a V_{H}having a CDR1 as shown in SEQ ID NO:30, a CDR2 as shown in SEQ ID NO:31 or SEQ ID NO:32 and a CDR3 as shown in SEQ ID NO:33 and a V_{L} having a CDR1 as shown in SEQ ID NO:34 or SEQ ID NO:35, a CDR2 as shown in SEQ ID NO:36 and a CDR3 as shown in SEQ ID NO: 37. For example, an Fv can include a V_{H}having a CDR1 as shown in SEQ ID NO:30, a CDR2 as shown in SEQ ID NO:31 and a CDR3 as shown in SEQ ID NO:33 and a V_{L} having a CDR1 as shown in SEQ ID NO:34, a CDR2 as shown in SEQ ID NO:36 and a CDR3 as shown in SEQ ID NO:37. In another embodiment, an Fv can include a V_{H} having a CDR1 as shown in SEQ ID NO:30, a CDR2 as shown in SEQ ID NO:31 and a CDR3 as shown in SEQ ID NO: 33 and a V_{L} having a CDR1 as shown in SEQ ID NO:35, a CDR2 as shown in SEQ ID NO: 36 and a CDR3 as shown in SEQ ID NO:37. In another embodiment, an Fv can include a V_{H}having a CDR1 as shown in SEQ ID NO:30, a CDR2 as shown in SEQ ID NO: 32 and a CDR3 as shown in SEQ ID NO:33 and a V_{L} having a CDR1 as shown in SEQ ID NO: 34, a CDR2 as shown in SEQ ID NO:36 and a CDR3 as shown in SEQ ID NO:37. In another embodiment, an Fv can include a V_{H}having a CDR1 as shown in SEQ ID NO:30, a CDR2 as shown in SEQ ID NO:32 and a CDR3 as shown in SEQ ID NO:33 and a V_{L} having a CDR1 as shown in SEQ ID NO:35, a CDR2 as shown in SEQ ID NO:36 and a CDR3 as shown in SEQ ID NO:37.

Above exemplified Fv may also have CDRs assigned using the IMGT system. Appropriate sequences from this system are referenced below.

In another embodiment, the Fv includes a V_{H} including a sequence at least 95% identical to the sequence as shown in any one of SEQ ID NOs: 9, 1, or 45 to 52 and a V_{L} including a sequence at least 95% identical to the sequence as shown in any one of SEQ ID NOs: 3 to 5, or 53 to 58. For example, an Fv can include a V_{H} including a sequence at least 95% identical to the sequence as shown in SEQ ID NO:47 and a V_{L} including a sequence at least 95% identical to the sequence as shown in SEQ ID NO:54. In another embodiment, an Fv can include a V_{H} including a sequence at least 95% identical to the sequence as shown in SEQ ID NO:50 and a V_{L} including a sequence at least 95% identical to the sequence as shown in SEQ ID NO:56. In another embodiment, an Fv can include a V_{H} including a sequence at least 95% identical to the sequence as shown in SEQ ID NO:52 and a V_{L} including a sequence at least 95% identical to the sequence as shown in SEQ ID NO:56. In these embodiments, the V_{H} and/or V_{L} can be at least 96%, at least 97%, at least 98% or at least 99% identical to the recited SEQ ID NO. In these embodiments, the Fv can have an above referenced combination of CDRs. For example, an Fv can include a V_{H} including a sequence at least 95% identical to the sequence as shown in SEQ ID NO:50 and a V_{L} including a sequence at least 95% identical to the sequence as shown in SEQ ID NO:56, wherein the V_{H} has a CDR1 as shown in SEQ ID NO:30, a CDR2 as shown in SEQ ID NO: 32 and a CDR3 as shown in SEQ ID NO:33 and the V_{L} has a CDR1 as shown in SEQ ID NO: 35, a CDR2 as shown in SEQ ID NO:36 and a CDR3 as shown in SEQ ID NO:37.

In another embodiment, the Fv includes a V_{H} including a sequence as shown in any one of SEQ ID NOs: 9, 11, or 45 to 52 and a V_{L} including a sequence as shown in any one of SEQ ID NOs: 3 to 5 or 53 to 58. For example, an Fv can include a V_{H} including a sequence as shown in SEQ ID NO:50 and a V_{L} including a sequence as shown in SEQ ID NO:56. In another embodiment, an Fv can include a V_{H} including a sequence as shown in SEQ ID NO: 52 and a V_{L} including a sequence as shown in SEQ ID NO:56.

In some embodiments, the V_{H} and V_{L} of the Fv can be in a single polypeptide chain. In another embodiment, the Fv lacks an Fc region. For example, the Fv can be a single chain Fv fragment (scFv), a dimeric scFv (di-scFv), a trimeric scFv (tri-scFv). In some embodiments, the Fv is an scFv. In another embodiment, the Fv is a di-scFv. In another embodiment, the Fv is a tri-scFv.

In another embodiment, the scFv, di-scFv or tri-scFv can be linked to a constant region of an antibody, Fc or a heavy chain constant domain CH2 and/or CH3.

In some embodiments, the present disclosure encompasses a cell-penetrating di-scFv having an antigen binding domain, wherein the antigen binding domain binds to or specifically binds to DNA.

In some embodiments, a di-scFv according to the present disclosure includes an amino acid sequence at least 95% identical to the sequence as shown in any one of SEQ ID NOs: 61 to 76. For example, the di-scFv includes an amino acid sequence at least 95% identical to the amino acid sequence shown in any one of SEQ ID NOs: 61, 65, 70 or 72. In these embodiments, amino acid sequences can be at least 96%, at least 97%, at least 98% or at least 99% identical to the recited SEQ ID NO. In some embodiments, a di-scFv according to the present disclosure includes an amino acid sequence as shown in any one of SEQ ID NOs: 61 to 76. For example, the di-scFv can include an amino acid sequence as shown in any one of SEQ ID NOs: 61, 65, 70 or 72.

In another embodiment, the V_{H} and V_{L} of the binding protein are in a separate polypeptide chain. For example, the binding protein can be a diabody, triabody, tetrabody, Fab, F(ab′) 2. In another embodiment, the binding protein can be an Fv which includes a V_{H} and V_{L} in separate polypeptide chains. In these embodiments, the binding proteins may be linked to a constant region of an antibody, Fc or a heavy chain constant domain CH2 and/or CH3.

In another embodiment, the binding protein can be an intact antibody. Accordingly, in some embodiments, the present disclosure encompasses an antibody having an antigen binding domain, wherein the antigen binding domain binds to or specifically binds to DNA. For example, the antibody can bind the same epitope as a binding protein having a V_{H} including an amino acid sequence as shown in SEQ ID NO:7 and a V_{L} including an amino acid sequence as shown in SEQ ID NO:2. In another embodiment, the antibody can bind the same epitope as a di-scFv having an amino acid sequence as shown in SEQ ID NO:28.

In another embodiment, the antibody includes a V_{H} having a CDR1 as shown in SEQ ID NO: 30, a CDR2 as shown in SEQ ID NO:31 or SEQ ID NO:32 and a CDR3 as shown in SEQ ID NO:33 and a V_{L} having a CDR1 as shown in SEQ ID NO:34 or SEQ ID NO:35, a CDR2 as shown in SEQ ID NO:36 and a CDR3 as shown in SEQ ID NO:37. For example, an antibody can include a V_{H}having a CDR1 as shown in SEQ ID NO:30, a CDR2 as shown in SEQ ID NO:31 and a CDR3 as shown in SEQ ID NO:33 and a V_{L} having a CDR1 as shown in SEQ ID NO:34, a CDR2 as shown in SEQ ID NO:36 and a CDR3 as shown in SEQ ID NO: 37. In another embodiment, an antibody can include a V_{H}having a CDR1 as shown in SEQ ID NO:30, a CDR2 as shown in SEQ ID NO:31 and a CDR3 as shown in SEQ ID NO: 33 and a V_{L} having a CDR1 as shown in SEQ ID NO:35, a CDR2 as shown in SEQ ID NO: 36 and a CDR3 as shown in SEQ ID NO:37. In another embodiment, an antibody can include a V_{H}having a CDR1 as shown in SEQ ID NO:30, a CDR2 as shown in SEQ ID NO: 32 and a CDR3 as shown in SEQ ID NO:33 and a V_{L} having a CDR1 as shown in SEQ ID NO: 34, a CDR2 as shown in SEQ ID NO:36 and a CDR3 as shown in SEQ ID NO:37. In another embodiment, an antibody can include a V_{H}having a CDR1 as shown in SEQ ID NO: 30, a CDR2 as shown in SEQ ID NO:32 and a CDR3 as shown in SEQ ID NO:33 and a V_{L} having a CDR1 as shown in SEQ ID NO:35, a CDR2 as shown in SEQ ID NO:36 and a CDR3 as shown in SEQ ID NO:37.

Above exemplified antibodies may also have CDRs assigned using the IMGT system. Appropriate sequences from this system are referenced below.

In another embodiment, the antibody includes a V_{H} including a sequence at least 95% identical to the sequence as shown in any one of SEQ ID NOs: 9, 11, or 45 to 52 and a V_{L} including a sequence at least 95% identical to the sequence as shown in any one of SEQ ID NOs: 3 to 5, or 53 to 58. For example, an antibody can include a V_{H} including a sequence at least 95% identical to the sequence as shown in SEQ ID NO:47 and a V_{L} including a sequence at least 95% identical to the sequence as shown in SEQ ID NO:54. In another embodiment, an antibody can include a V_{H} including a sequence at least 95% identical to the sequence as shown in SEQ ID NO:50 and a V_{L} including a sequence at least 95% identical to the sequence as shown in SEQ ID NO:56. In another embodiment, an antibody can include a V_{H} including a sequence at least 95% identical to the sequence as shown in SEQ ID NO:52 and a V_{L} including a sequence at least 95% identical to the sequence as shown in SEQ ID NO: 56. In these embodiments, the V_{H} and/or V_{L} can be at least 96%, at least 97%, at least 98% or at least 99% identical to the recited SEQ ID NO. In these embodiments, the antibody can have an above referenced combination of CDRs. For example, an antibody can include a V_{H} including a sequence at least 95% identical to the sequence as shown in SEQ ID NO:50 and a V_{L} including a sequence at least 95% identical to the sequence as shown in SEQ ID NO: 56, wherein the V_{H} has a CDR1 as shown in SEQ ID NO:30, a CDR2 as shown in SEQ ID NO: 32 and a CDR3 as shown in SEQ ID NO:33 and the V_{L} has a CDR1 as shown in SEQ ID NO: 35, a CDR2 as shown in SEQ ID NO:36 and a CDR3 as shown in SEQ ID NO:37.

In another embodiment, the antibody includes a V_{H} including a sequence as shown in any one of SEQ ID NOs: 9, 11, or 45 to 52 and a V_{L} including a sequence as shown in any one of SEQ ID NOs: 3 to 5, or 53 to 58. For example, an antibody can include a V_{H} including a sequence as shown in SEQ ID NO:47 and a V_{L} including a sequence as shown in SEQ ID NO: 54. In another embodiment, an antibody can include a V_{H} including a sequence as shown in SEQ ID NO:50 and a V_{L} including a sequence as shown in SEQ ID NO:56. In another embodiment, an antibody can include a V_{H} including a sequence as shown in SEQ ID NO:52 and a V_{L} including a sequence as shown in SEQ ID NO:56.

In another embodiment, the antibody has an amino acid sequence shown in any one of SEQ ID NOs: 77, 82 or 84 and an amino acid sequence shown in SEQ ID NO:87.

Exemplary sequences from anti-DNA binding protein sequences encompassed by the present disclosure follow:

Heavy Chain CDR1 KABAT
SEQ ID NO: 30
NYGMH
Heavy Chain CDR2 (variants 2-4, 6-8, 10-12)
KABAT
SEQ ID NO: 31
YISSSSSTIYYADSVKG
Heavy Chain CDR2 (variants 13-19) KABAT
SEQ ID NO: 32
YISSGSSTIYYADSVKG
Heavy Chain CDR3 KABAT
SEQ ID NO: 33
RGLLLDY
Light Chain CDR1 (variants 2-4, 6-8, 10-12)
KABAT
SEQ ID NO: 34
RASKSVSTSSYSYMH
Light Chain CDR1 (variants 13-19) KABAT
SEQ ID NO: 35
RASKTVSTSSYSYMH
Light Chain CDR2 KABAT
SEQ ID NO: 36
YASYLES
Light Chain CDR3 KABAT
SEQ ID NO: 37
QHSREFPWT
Heavy Chain CDR1 IMGT
SEQ ID NO: 38
GFTFSNYG
Heavy Chain CDR2 (variants 2-4, 6-8, 10-12)
IMGT
SEQ ID NO: 39
ISSSSSTI
Heavy Chain CDR2 (variants 13-19) IMGT
SEQ ID NO: 40
ISSGSSTI
Heavy Chain CDR3 IMGT
SEQ ID NO: 41
ARRGLLLDY
Light Chain CDR1 (variants 2-4, 6-8, 10-12)
IMGT
SEQ ID NO: 42
KSVSTSSYSY
Light Chain CDR1 (variants 13-19) IMGT
SEQ ID NO: 43
KTVSTSSYSY
Light Chain CDR2 IMGT
SEQ ID NO: 44
YAS
Light Chain CDR3 IMGT
SEQ ID NO: 37
QHSREFPWT
Heavy Chain variable region (variants 2, 6
and 10)
SEQ ID NO: 46
EVQLVESGGGLVQPGGSLRLSCAASGFTFSNYGMHWVRQAPGKGLEWVSY
ISSSSSTIYYADSVKGRFTISRDNAKNSLYLQMNSLRAEDTAVYYCARRG
LLLDYWGQGTTVTVSS
Heavy Chain variable region (variants 3, 7
and 11)
SEQ ID NO: 47
EVQLVESGGGVVQPGGSLRLSCAASGFTFSNYGMHWVRQAPEKGLEWVSY
ISSSSSTIYYADSVKGRFTISRDNSKNTLYLQMNSLRAEDTAVYYCARRG
LLLDYWGQGTTVTVSS
Heavy Chain variable region (variants 4, 8
and 12)
SEQ ID NO: 48
EVQLVESGGGDVKPGGSLRLSCAASGFTFSNYGMHWVRQAPEKGLEWVSY
ISSSSSTIYYADSVKGRFTISRDNSKNTLYLQMNSLRAEDTAVYYCARRG
LLLDYWGQGTTVTVSS
Heavy Chain variable region (variants 13, 16
and 19)
SEQ ID NO: 50
EVQLVESGGGLVQPGGSLRLSCAASGFTFSNYGMHWVRQAPGKGLEWVSY
ISSGSSTIYYADSVKGRFTISRDNAKNSLYLQMNSLRAEDTAVYYCARRG
LLLDYWGQGTTVTVSS
Heavy Chain variable region (variants 14
and 17)
SEQ ID NO: 51
EVQLVESGGGVVQPGGSLRLSCAASGFTFSNYGMHWVRQAPEKGLEWVSY
ISSGSSTIYYADSVKGRFTISRDNSKNTLYLQMNSLRAEDTAVYYCARRG
LLLDYWGQGTTVTVSS
Heavy Chain variable region (variants 15
and 18)
SEQ ID NO: 52
EVQLVESGGGDVKPGGSLRLSCAASGFTFSNYGMHWVRQAPEKGLEWVSY
ISSGSSTIYYADSVKGRFTISRDNSKNTLYLQMNSLRAEDTAVYYCARRG
LLLDYWGQGTTVTVSS
Heavy Chain variable region (hVH1,
WO2016/033324)
SEQ ID NO: 9
EVQLVQSGGGLIQPGGSLRLSCAASGFTFSNYGMHWVRQAPGKGLEWVSY
ISSGSSTIYYADSVKGRFTISRDNSKNTLYLQMNSLRAEDTAVYYCARRG
LLLDYWGQGTTVTVSS
Heavy Chain variable region (hVH2,
WO2016/033324)
SEQ ID NO: 11
EVQLVESGGGLIQPGGSLRLSCAASGFTFSNYGMHWVRQAPGKGLEWVSY
ISSGSSTIYYADSVKGRFTISRDNSKNTLYLQMTSLRAEDTAVYYCARRG
LLLDYWGQGTTLTVSS
Heavy Chain variable region (hVH3,
WO2016/033324)
SEQ ID NO: 45
EVQLQESGGGVVQPGGSLRLSCAASGFTFSNYGMHWIRQAPGKGLEWVSY
ISSGSSTIYYADSVKGRFTISRDNSKNTLYLQMNSLRSEDTAVYYCARRG
LLLDYWGQGTLVTVSS
Heavy Chain variable region (hVH4,
WO2016/033324)
SEQ ID NO: 49
EVQLVESGGGLVQPGGSLRLSCSASGFTFSNYGMHWVRQAPGKGLEYVSY
ISSGSSTIYYADTVKGRFTISRDNSKNTLYLQMSSLRAEDTAVYYCVKRG
LLLDYWGQGTLVTVSS
Light Chain variable region (variants 2, 3
and 4)
SEQ ID NO: 53
DIQMTQSPSSLSASLGDRATITCRASKSVSTSSYSYMHWYQQKPGQPPKL
LIKYASYLESGVPSRFSGSGSGTDFTLTISSLQPEDAATYYCQHSREFPW
TFGGGTKVEIK
Light Chain variable region (variants 6, 7
and 8)
SEQ ID NO: 54
DIQMTQSPSSLSASLGDRATITCRASKSVSTSSYSYMHWYQQKPGQAPKL
LIKYASYLESGVPSRFSGSGSGTDFTLTISSLQPEDFATYYCQHSREFPW
TFGQGTKVEIK
Light Chain variable region (variants 10, 11
and 12)
SEQ ID NO: 55
DIQMTQSPSSLSASVGDRVTITCRASKSVSTSSYSYMHWYQQKPGKAPKL
LIKYASYLESGVPSRFSGSGSGTDFTLTISSLQPEDFATYYCQHSREFPW
TFGQGTKVEIK
Light Chain variable region (variants 13, 14
and 15)
SEQ ID NO: 56
DIQMTQSPSSLSASLGDRATITCRASKTVSTSSYSYMHWYQQKPGQPPKL
LIKYASYLESGVPSRFSGSGSGTDFTLTISSLQPEDAATYYCQHSREFPW
TFGGGTKVEIK
Light Chain variable region (variants 16, 17
and 18)
SEQ ID NO: 57
DIQMTQSPSSLSASVGDRVTITCRASKTVSTSSYSYMHWYQQKPGKAPKL
LIKYASYLESGVPSRFSGSGSGTDFTLTISSLQPEDFATYYCQHSREFPW
TFGQGTKVEIK
Light Chain variable region (variant 19)
SEQ ID NO: 58
DIQMTQSPSSLSASLGDRATITCRASKTVSTSSYSYMHWYQQKPGQAPKL
LIKYASYLESGVPSRFSGSGSGTDFTLTISSLQPEDFATYYCQHSREFPW
TFGQGTKVEIK
Light Chain variable region (hVL1,
WO2016/033324)
SEQ ID NO: 3
DIQMTQSPSSLSASVGDRVTITCRASKSVSTSSYSYLAWYQQKPEKAPKL
LIKYASYLQSGVPSRFSGSGSGTDFTLTISSLQPEDFATYYCQHSREFPW
TFGAGTKLELK
Light Chain variable region (hVL2,
WO2016/033324)
SEQ ID NO: 4
DIQMTQSPSSLSASVGDRVTISCRASKSVSTSSYSYMHWYQQKPEKAPKL
LIKYASYLQSGVPSRFSGSGSGTDFTLTISSLQPEDVATYYCQHSREFPW
TFGAGTKLELK
Light Chain variable region (hVL3,
WO2016/033324)
SEQ ID NO: 5
DIVLTQSPASLAVSPGQRATITCRASKSVSTSSYSYMHWYQQKPGQPPKL
LIYYASYLESGVPARFSGSGSGTDFTLTINPVEANDTANYYCQHSREFPW
TFGQGTKVEIK
Linker sequence 1
SEQ ID NO: 59
RADAAPGGGGSGGGGSGGGGS
Linker sequence 2
SEQ ID NO: 60
ASTKGPSVFPLAPLESSGS
Variant 2
SEQ ID NO: 61
DIQMTQSPSSLSASLGDRATITCRASKSVSTSSYSYMHWYQQKPGQPPKL
LIKYASYLESGVPSRFSGSGSGTDFTLTISSLQPEDAATYYCQHSREFPW
TFGGGTKVEIKRADAAPGGGGSGGGGSGGGGSEVQLVESGGGLVQPGGSL
RLSCAASGFTFSNYGMHWVRQAPGKGLEWVSYISSSSSTIYYADSVKGRF
TISRDNAKNSLYLQMNSLRAEDTAVYYCARRGLLLDYWGQGTTVTVSSAS
TKGPSVFPLAPLESSGSDIQMTQSPSSLSASLGDRATITCRASKSVSTSS
YSYMHWYQQKPGQPPKLLIKYASYLESGVPSRFSGSGSGTDFTLTISSLQ
PEDAATYYCQHSREFPWTFGGGTKVEIKRADAAPGGGGSGGGGSGGGGSE
VQLVESGGGLVQPGGSLRLSCAASGFTFSNYGMHWVRQAPGKGLEWVSYI
SSSSSTIYYADSVKGRFTISRDNAKNSLYLQMNSLRAEDTAVYYCARRGL
LLDYWGQGTTVTVSS
Variant 3
SEQ ID NO: 62
DIQMTQSPSSLSASLGDRATITCRASKSVSTSSYSYMHWYQQKPGQPPKL
LIKYASYLESGVPSRFSGSGSGTDFTLTISSLQPEDAATYYCQHSREFPW
TFGGGTKVEIKRADAAPGGGGSGGGGSGGGGSEVQLVESGGGVVQPGGSL
RLSCAASGFTFSNYGMHWVRQAPEKGLEWVSYISSSSSTIYYADSVKGRF
TISRDNSKNTLYLQMNSLRAEDTAVYYCARRGLLLDYWGQGTTVTVSSAS
TKGPSVFPLAPLESSGSDIQMTQSPSSLSASLGDRATITCRASKSVSTSS
YSYMHWYQQKPGQPPKLLIKYASYLESGVPSRFSGSGSGTDFTLTISSLQ
PEDAATYYCQHSREFPWTFGGGTKVEIKRADAAPGGGGSGGGGSGGGGSE
VQLVESGGGVVQPGGSLRLSCAASGFTFSNYGMHWVRQAPEKGLEWVSYI
SSSSSTIYYADSVKGRFTISRDNSKNTLYLQMNSLRAEDTAVYYCARRGL
LLDYWGQGTTVTVSS
Variant 4
SEQ ID NO: 63
DIQMTQSPSSLSASLGDRATITCRASKSVSTSSYSYMHWYQQKPGQPPKL
LIKYASYLESGVPSRFSGSGSGTDFTLTISSLQPEDAATYYCQHSREFPW
TFGGGTKVEIKRADAAPGGGGSGGGGSGGGGSEVQLVESGGGDVKPGGSL
RLSCAASGFTFSNYGMHWVRQAPEKGLEWVSYISSSSSTIYYADSVKGRF
TISRDNSKNTLYLQMNSLRAEDTAVYYCARRGLLLDYWGQGTTVTVSSAS
TKGPSVFPLAPLESSGSDIQMTQSPSSLSASLGDRATITCRASKSVSTSS
YSYMHWYQQKPGQPPKLLIKYASYLESGVPSRFSGSGSGTDFTLTISSLQ
PEDAATYYCQHSREFPWTFGGGTKVEIKRADAAPGGGGSGGGGSGGGGSE
VQLVESGGGDVKPGGSLRLSCAASGFTFSNYGMHWVRQAPEKGLEWVSYI
SSSSSTIYYADSVKGRFTISRDNSKNTLYLQMNSLRAEDTAVYYCARRGL
LLDYWGQGTTVTVSS
Variant 6
SEQ ID NO: 64
DIQMTQSPSSLSASLGDRATITCRASKSVSTSSYSYMHWYQQKPGQAPKL
LIKYASYLESGVPSRFSGSGSGTDFTLTISSLQPEDFATYYCQHSREFPW
TFGQGTKVEIKRADAAPGGGGSGGGGSGGGGSEVQLVESGGGLVQPGGSL
RLSCAASGFTFSNYGMHWVRQAPGKGLEWVSYISSSSSTIYYADSVKGRF
TISRDNAKNSLYLQMNSLRAEDTAVYYCARRGLLLDYWGQGTTVTVSSAS
TKGPSVFPLAPLESSGSDIQMTQSPSSLSASLGDRATITCRASKSVSTSS
YSYMHWYQQKPGQAPKLLIKYASYLESGVPSRFSGSGSGTDFTLTISSLQ
PEDFATYYCQHSREFPWTFGQGTKVEIKRADAAPGGGGSGGGGSGGGGSE
VQLVESGGGLVQPGGSLRLSCAASGFTFSNYGMHWVRQAPGKGLEWVSYI
SSSSSTIYYADSVKGRFTISRDNAKNSLYLQMNSLRAEDTAVYYCARRGL
LLDYWGQGTTVTVSS
Variant 7
SEQ ID NO: 65
DIQMTQSPSSLSASLGDRATITCRASKSVSTSSYSYMHWYQQKPGQAPKL
LIKYASYLESGVPSRFSGSGSGTDFTLTISSLQPEDFATYYCQHSREFPW
TFGQGTKVEIKRADAAPGGGGSGGGGSGGGGSEVQLVESGGGVVQPGGSL
RLSCAASGFTFSNYGMHWVRQAPEKGLEWVSYISSSSSTIYYADSVKGRF
TISRDNSKNTLYLQMNSLRAEDTAVYYCARRGLLLDYWGQGTTVTVSSAS
TKGPSVFPLAPLESSGSDIQMTQSPSSLSASLGDRATITCRASKSVSTSS
YSYMHWYQQKPGQAPKLLIKYASYLESGVPSRFSGSGSGTDFTLTISSLQ
PEDFATYYCQHSREFPWTFGQGTKVEIKRADAAPGGGGSGGGGSGGGGSE
VQLVESGGGVVQPGGSLRLSCAASGFTFSNYGMHWVRQAPEKGLEWVSYI
SSSSSTIYYADSVKGRFTISRDNSKNTLYLQMNSLRAEDTAVYYCARRGL
LLDYWGQGTTVTVSS
Variant 8
SEQ ID NO: 66
DIQMTQSPSSLSASLGDRATITCRASKSVSTSSYSYMHWYQQKPGQAPKL
LIKYASYLESGVPSRFSGSGSGTDFTLTISSLQPEDFATYYCQHSREFPW
TFGQGTKVEIKRADAAPGGGGSGGGGSGGGGSEVQLVESGGGDVKPGGSL
RLSCAASGFTFSNYGMHWVRQAPEKGLEWVSYISSSSSTIYYADSVKGRF
TISRDNSKNTLYLQMNSLRAEDTAVYYCARRGLLLDYWGQGTTVTVSSAS
TKGPSVFPLAPLESSGSDIQMTQSPSSLSASLGDRATITCRASKSVSTSS
YSYMHWYQQKPGQAPKLLIKYASYLESGVPSRFSGSGSGTDFTLTISSLQ
PEDFATYYCQHSREFPWTFGQGTKVEIKRADAAPGGGGSGGGGSGGGGSE
VQLVESGGGDVKPGGSLRLSCAASGFTFSNYGMHWVRQAPEKGLEWVSYI
SSSSSTIYYADSVKGRFTISRDNSKNTLYLQMNSLRAEDTAVYYCARRGL
LLDYWGQGTTVTVSS
Variant 10
SEQ ID NO: 67
DIQMTQSPSSLSASVGDRVTITCRASKSVSTSSYSYMHWYQQKPGKAPKL
LIKYASYLESGVPSRFSGSGSGTDFTLTISSLQPEDFATYYCQHSREFPW
TFGQGTKVEIKRADAAPGGGGSGGGGSGGGGSEVQLVESGGGLVQPGGSL
RLSCAASGFTFSNYGMHWVRQAPGKGLEWVSYISSSSSTIYYADSVKGRF
TISRDNAKNSLYLQMNSLRAEDTAVYYCARRGLLLDYWGQGTTVTVSSAS
TKGPSVFPLAPLESSGSDIQMTQSPSSLSASVGDRVTITCRASKSVSTSS
YSYMHWYQQKPGKAPKLLIKYASYLESGVPSRFSGSGSGTDFTLTISSLQ
PEDFATYYCQHSREFPWTFGQGTKVEIKRADAAPGGGGSGGGGSGGGGSE
VQLVESGGGLVQPGGSLRLSCAASGFTFSNYGMHWVRQAPGKGLEWVSYI
SSSSSTIYYADSVKGRFTISRDNAKNSLYLQMNSLRAEDTAVYYCARRGL
LLDYWGQGTTVTVSS
Variant 11
SEQ ID NO: 68
DIQMTQSPSSLSASVGDRVTITCRASKSVSTSSYSYMHWYQQKPGKAPKL
LIKYASYLESGVPSRFSGSGSGTDFTLTISSLQPEDFATYYCQHSREFPW
TFGQGTKVEIKRADAAPGGGGSGGGGSGGGGSEVQLVESGGGVVQPGGSL
RLSCAASGFTFSNYGMHWVRQAPEKGLEWVSYISSSSSTIYYADSVKGRF
TISRDNSKNTLYLQMNSLRAEDTAVYYCARRGLLLDYWGQGTTVTVSSAS
TKGPSVFPLAPLESSGSDIQMTQSPSSLSASVGDRVTITCRASKSVSTSS
YSYMHWYQQKPGKAPKLLIKYASYLESGVPSRFSGSGSGTDFTLTISSLQ
PEDFATYYCQHSREFPWTFGQGTKVEIKRADAAPGGGGSGGGGSGGGGSE
VQLVESGGGVVQPGGSLRLSCAASGFTFSNYGMHWVRQAPEKGLEWVSYI
SSSSSTIYYADSVKGRFTISRDNSKNTLYLQMNSLRAEDTAVYYCARRGL
LLDYWGQGTTVTVSS
Variant 12
SEQ ID NO: 69
DIQMTQSPSSLSASVGDRVTITCRASKSVSTSSYSYMHWYQQKPGKAPKL
LIKYASYLESGVPSRFSGSGSGTDFTLTISSLQPEDFATYYCQHSREFPW
TFGQGTKVEIKRADAAPGGGGSGGGGSGGGGSEVQLVESGGGDVKPGGSL
RLSCAASGFTFSNYGMHWVRQAPEKGLEWVSYISSSSSTIYYADSVKGRF
TISRDNSKNTLYLQMNSLRAEDTAVYYCARRGLLLDYWGQGTTVTVSSAS
TKGPSVFPLAPLESSGSDIQMTQSPSSLSASVGDRVTITCRASKSVSTSS
YSYMHWYQQKPGKAPKLLIKYASYLESGVPSRFSGSGSGTDFTLTISSLQ
PEDFATYYCQHSREFPWTFGQGTKVEIKRADAAPGGGGSGGGGSGGGGSE
VQLVESGGGDVKPGGSLRLSCAASGFTFSNYGMHWVRQAPEKGLEWVSYI
SSSSSTIYYADSVKGRFTISRDNSKNTLYLQMNSLRAEDTAVYYCARRGL
LLDYWGQGTTVTVSS
Variant 13
SEQ ID NO: 70
DIQMTQSPSSLSASLGDRATITCRASKTVSTSSYSYMHWYQQKPGQPPKL
LIKYASYLESGVPSRFSGSGSGTDFTLTISSLQPEDAATYYCQHSREFPW
TFGGGTKVEIKRADAAPGGGGSGGGGSGGGGSEVQLVESGGGLVQPGGSL
RLSCAASGFTFSNYGMHWVRQAPGKGLEWVSYISSGSSTIYYADSVKGRF
TISRDNAKNSLYLQMNSLRAEDTAVYYCARRGLLLDYWGQGTTVTVSSAS
TKGPSVFPLAPLESSGSDIQMTQSPSSLSASLGDRATITCRASKTVSTSS
YSYMHWYQQKPGQPPKLLIKYASYLESGVPSRFSGSGSGTDFTLTISSLQ
PEDAATYYCQHSREFPWTFGGGTKVEIKRADAAPGGGGSGGGGSGGGGSE
VQLVESGGGLVQPGGSLRLSCAASGFTFSNYGMHWVRQAPGKGLEWVSYI
SSGSSTIYYADSVKGRFTISRDNAKNSLYLQMNSLRAEDTAVYYCARRGL
LLDYWGQGTTVTVSS
Variant 14
SEQ ID NO: 71
DIQMTQSPSSLSASLGDRATITCRASKTVSTSSYSYMHWYQQKPGQPPKL
LIKYASYLESGVPSRFSGSGSGTDFTLTISSLQPEDAATYYCQHSREFPW
TFGGGTKVEIKRADAAPGGGGSGGGGSGGGGSEVQLVESGGGVVQPGGSL
RLSCAASGFTFSNYGMHWVRQAPEKGLEWVSYISSGSSTIYYADSVKGRF
TISRDNSKNTLYLQMNSLRAEDTAVYYCARRGLLLDYWGQGTTVTVSSAS
TKGPSVFPLAPLESSGSDIQMTQSPSSLSASLGDRATITCRASKTVSTSS
YSYMHWYQQKPGQPPKLLIKYASYLESGVPSRFSGSGSGTDFTLTISSLQ
PEDAATYYCQHSREFPWTFGGGTKVEIKRADAAPGGGGSGGGGSGGGGSE
VQLVESGGGVVQPGGSLRLSCAASGFTFSNYGMHWVRQAPEKGLEWVSYI
SSGSSTIYYADSVKGRFTISRDNSKNTLYLQMNSLRAEDTAVYYCARRGL
LLDYWGQGTTVTVSS
Variant 15
SEQ ID NO: 72
DIQMTQSPSSLSASLGDRATITCRASKTVSTSSYSYMHWYQQKPGQPPKL
LIKYASYLESGVPSRFSGSGSGTDFTLTISSLQPEDAATYYCQHSREFPW
TFGGGTKVEIKRADAAPGGGGSGGGGSGGGGSEVQLVESGGGDVKPGGSL
RLSCAASGFTFSNYGMHWVRQAPEKGLEWVSYISSGSSTIYYADSVKGRF
TISRDNSKNTLYLQMNSLRAEDTAVYYCARRGLLLDYWGQGTTVTVSSAS
TKGPSVFPLAPLESSGSDIQMTQSPSSLSASLGDRATITCRASKTVSTSS
YSYMHWYQQKPGQPPKLLIKYASYLESGVPSRFSGSGSGTDFTLTISSLQ
PEDAATYYCQHSREFPWTFGGGTKVEIKRADAAPGGGGSGGGGSGGGGSE
VQLVESGGGDVKPGGSLRLSCAASGFTFSNYGMHWVRQAPEKGLEWVSYI
SSGSSTIYYADSVKGRFTISRDNSKNTLYLQMNSLRAEDTAVYYCARRGL
LLDYWGQGTTVTVSS
Variant 16
SEQ ID NO: 73
DIQMTQSPSSLSASVGDRVTITCRASKTVSTSSYSYMHWYQQKPGKAPKL
LIKYASYLESGVPSRFSGSGSGTDFTLTISSLQPEDFATYYCQHSREFPW
TFGQGTKVEIKRADAAPGGGGSGGGGSGGGGSEVQLVESGGGLVQPGGSL
RLSCAASGFTFSNYGMHWVRQAPGKGLEWVSYISSGSSTIYYADSVKGRF
TISRDNAKNSLYLQMNSLRAEDTAVYYCARRGLLLDYWGQGTTVTVSSAS
TKGPSVFPLAPLESSGSDIQMTQSPSSLSASVGDRVTITCRASKTVSTSS
YSYMHWYQQKPGKAPKLLIKYASYLESGVPSRFSGSGSGTDFTLTISSLQ
PEDFATYYCQHSREFPWTFGQGTKVEIKRADAAPGGGGSGGGGSGGGGSE
VQLVESGGGLVQPGGSLRLSCAASGFTFSNYGMHWVRQAPGKGLEWVSYI
SSGSSTIYYADSVKGRFTISRDNAKNSLYLQMNSLRAEDTAVYYCARRGL
LLDYWGQGTTVTVSS
Variant 17
SEQ ID NO: 74
DIQMTQSPSSLSASVGDRVTITCRASKTVSTSSYSYMHWYQQKPGKAPKL
LIKYASYLESGVPSRFSGSGSGTDFTLTISSLQPEDFATYYCQHSREFPW
TFGQGTKVEIKRADAAPGGGGSGGGGSGGGGSEVQLVESGGGVVQPGGSL
RLSCAASGFTFSNYGMHWVRQAPEKGLEWVSYISSGSSTIYYADSVKGRF
TISRDNSKNTLYLQMNSLRAEDTAVYYCARRGLLLDYWGQGTTVTVSSAS
TKGPSVFPLAPLESSGSDIQMTQSPSSLSASVGDRVTITCRASKTVSTSS
YSYMHWYQQKPGKAPKLLIKYASYLESGVPSRFSGSGSGTDFTLTISSLQ
PEDFATYYCQHSREFPWTFGQGTKVEIKRADAAPGGGGSGGGGSGGGGSE
VQLVESGGGVVQPGGSLRLSCAASGFTFSNYGMHWVRQAPEKGLEWVSYI
SSGSSTIYYADSVKGRFTISRDNSKNTLYLQMNSLRAEDTAVYYCARRGL
LLDYWGQGTTVTVSS
Variant 18
SEQ ID NO: 75
DIQMTQSPSSLSASVGDRVTITCRASKTVSTSSYSYMHWYQQKPGKAPKL
LIKYASYLESGVPSRFSGSGSGTDFTLTISSLQPEDFATYYCQHSREFPW
TFGQGTKVEIKRADAAPGGGGSGGGGSGGGGSEVQLVESGGGDVKPGGSL
RLSCAASGFTFSNYGMHWVRQAPEKGLEWVSYISSGSSTIYYADSVKGRF
TISRDNSKNTLYLQMNSLRAEDTAVYYCARRGLLLDYWGQGTTVTVSSAS
TKGPSVFPLAPLESSGSDIQMTQSPSSLSASVGDRVTITCRASKTVSTSS
YSYMHWYQQKPGKAPKLLIKYASYLESGVPSRFSGSGSGTDFTLTISSLQ
PEDFATYYCQHSREFPWTFGQGTKVEIKRADAAPGGGGSGGGGSGGGGSE
VQLVESGGGDVKPGGSLRLSCAASGFTFSNYGMHWVRQAPEKGLEWVSYI
SSGSSTIYYADSVKGRFTISRDNSKNTLYLQMNSLRAEDTAVYYCARRGL
LLDYWGQGTTVTVSS
Variant 19
SEQ ID NO: 76
DIQMTQSPSSLSASLGDRATITCRASKTVSTSSYSYMHWYQQKPGQAPKL
LIKYASYLESGVPSRFSGSGSGTDFTLTISSLQPEDFATYYCQHSREFPW
TFGQGTKVEIKRADAAPGGGGSGGGGSGGGGSEVQLVESGGGLVQPGGSL
RLSCAASGFTFSNYGMHWVRQAPGKGLEWVSYISSGSSTIYYADSVKGRF
TISRDNAKNSLYLQMNSLRAEDTAVYYCARRGLLLDYWGQGTTVTVSSAS
TKGPSVFPLAPLESSGSDIQMTQSPSSLSASLGDRATITCRASKTVSTSS
YSYMHWYQQKPGQAPKLLIKYASYLESGVPSRFSGSGSGTDFTLTISSLQ
PEDFATYYCQHSREFPWTFGQGTKVEIKRADAAPGGGGSGGGGSGGGGSE
VQLVESGGGLVQPGGSLRLSCAASGFTFSNYGMHWVRQAPGKGLEWVSYI
SSGSSTIYYADSVKGRFTISRDNAKNSLYLQMNSLRAEDTAVYYCARRGL
LLDYWGQGTTVTVSS
In another embodiment, a humanized Fv3E10
includes
(Fv3E10, WO2016/033324))
SEQ ID NO: 88
DIVLTQSPASLAVSPGQRATITCRASKSVSTSSYSYMHWYQQKPGQPPKL
LIYYASYLESGVPARFSGSGSGTDFTLTINPVEANDTANYYCQHSREFPW
TFGQGTKVEIKGGGGSGGGGSGGGGSEVQLVESGGGLVQPGGSLRLSCSA
SGFTFSNYGMHWVRQAPGKGLEYVSYISSGSSTIYYADTVKGRFTISRDN
SKNTLYLQMSSLRAEDTAVYYCVKRGLLLDYWGQGTLVTVSS
IgG1 L2345A/L235A heavy chain full length
sequence
SEQ ID NO: 77
EVQLVESGGGLVQPGGSLRLSCAASGFTFSNYGMHWVRQAPGKGLEWVSY
ISSGSSTIYYADSVKGRFTISRDNAKNSLYLQMNSLRAEDTAVYYCARRG
LLLDYWGQGTTVTVSSASTKGPSVFPLAPSSKSTSGGTAALGCLVKDYFP
EPVTVSWNSGALTSGVHTFPAVLQSSGLYSLSSVVTVPSSSLGTQTYICN
VNHKPSNTKVDKKVEPKSCDKTHTCPPCPAPEAAGGPSVFLFPPKPKDTL
MISRTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVYTL
PPSRDELTKNQVSLTCLVKGFYPSDIAVEWESNGQPENNYKTTPPVLDSD
GSFFLYSKLTVDKSRWQQGNVFSCSVMHEALHNHYTQKSLSLSPGK
IgG1 constant heavy region 1
SEQ ID NO: 78
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKV
IgG1 hinge region
SEQ ID NO: 79
EPKSCDKTHTCP
IgG1 L2345A/L235A constant heavy region 2
SEQ ID NO: 80
PCPAPEAAGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKA
LPAPIEKTISKAK
IgG1 constant heavy region 3
SEQ ID NO: 81
GQPREPQVYTLPPSRDELTKNQVSLTCLVKGFYPSDIAVEWESNGQPENN
YKTTPPVLDSDGSFFLYSKLTVDKSRWQQGNVFSCSVMHEALHNHYTQKS
LSLSPGK
IgG1 N297D heavy chain full length sequence
SEQ ID NO: 82
EVQLVESGGGLVQPGGSLRLSCAASGFTFSNYGMHWVRQAPGKGLEWVSY
ISSGSSTIYYADSVKGRFTISRDNAKNSLYLQMNSLRAEDTAVYYCARRG
LLLDYWGQGTTVTVSSASTKGPSVFPLAPSSKSTSGGTAALGCLVKDYFP
EPVTVSWNSGALTSGVHTFPAVLQSSGLYSLSSVVTVPSSSLGTQTYICN
VNHKPSNTKVDKKVEPKSCDKTHTCPPCPAPELLGGPSVFLFPPKPKDTL
MISRTPEVTCVVVDVSHEDPEVKFNWYVDGVEVHNAKTKPREEQYDSTYR
VVSVLTVLHQDWLNGKEYKCKVSNKALPAPIEKTISKAKGQPREPQVYTL
PPSRDELTKNQVSLTCLVKGFYPSDIAVEWESNGQPENNYKTTPPVLDSD
GSFFLYSKLTVDKSRWQQGNVFSCSVMHEALHNHYTQKSLSLSPGK
IgG1 N297D constant heavy region 2
SEQ ID NO: 83
PCPAPELLGGPSVFLFPPKPKDTLMISRTPEVTCVVVDVSHEDPEVKFNW
YVDGVEVHNAKTKPREEQYDSTYRVVSVLTVLHQDWLNGKEYKCKVSNKA
LPAPIEKTISKAK
IgG1 L2345A/L235A/N297D heavy chain full
length sequence
SEQ ID NO: 84
EVQLVESGGGLVQPGGSLRLSCAASGFTFSNYGMHWVRQAPGKGLEWVSY
ISSGSSTIYYADSVKGRFTISRDNAKNSLYLQMNSLRAEDTAVYYCARRG
LLLDYWGQGTTVTVSSASTKGPSVFPLAPSSKSTSGGTAALGCLVKDYFP
EPVTVSWNSGALTSGVHTFPAVLQSSGLYSLSSVVTVPSSSLGTQTYICN
VNHKPSNTKVDKKVEPKSCDKTHTCPPCPAPEAAGGPSVFLFPPKPKDTL
MISRTPEVTCVVVDVSHEDPEVKFNWYVDGVEVHNAKTKPREEQYDSTYR
VVSVLTVLHQDWLNGKEYKCKVSNKALPAPIEKTISKAKGQPREPQVYTL
PPSRDELTKNQVSLTCLVKGFYPSDIAVEWESNGQPENNYKTTPPVLDSD
GSFFLYSKLTVDKSRWQQGNVFSCSVMHEALHNHYTQKSLSLSPGK
IgG1 L2345A/L235A/N297D constant heavy
region 2
SEQ ID NO: 85
PCPAPEAAGGPSVFLFPPKPKDTLMISRTPEVTCVVVDVSHEDPEVKFNW
YVDGVEVHNAKTKPREEQYDSTYRVVSVLTVLHQDWLNGKEYKCKVSNKA
LPAPIEKTISKAK
Unmodified constant heavy region 2
SEQ ID NO: 86
PCPAPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKA
LPAPIEKTISKAK
Light chain full length sequence
SEQ ID NO: 87
DIQMTQSPSSLSASLGDRATITCRASKTVSTSSYSYMHWYQQKPGQPPKL
LIKYASYLESGVPSRFSGSGSGTDFTLTISSLQPEDAATYYCQHSREFPW
TFGGGTKLEIKRTVAAPSVFIFPPSDEQLKSGTASVVCLLNNFYPREAKV
QWKVDNALQSGNSQESVTEQDSKDSTYSLSSTLTLSKADYEKHKVYACEV
THQGLSSPVTKSFNRGEC

### f. Additional Exemplary anti-DNA scFv Sequences

Exemplary murine 3E10 scFv sequences, including mono-, di-, and tri-scFv are disclosed in WO 2016/033321 and WO 2017/218825 and provided below. Cell-penetrating antibodies for use in the disclosed combination therapies include exemplary scFv, and fragments and variants thereof.

The amino acid sequence for scFv 3E10 (D31N) is:

(SEQ ID NO: 27)
AGIHDIVLTQSPASLAVSLGQRATISCRASKSVSTSSYSYMHWYQQKPGQ
PPKLLIKYASYLESGVPARFSGSGSGTDFTLNIHPVEEEDAATYYCQHSR
EFPWTFGGGTKLEIKRADAAPGGGGSGGGGSGGGGSEVQLVESGGGLVKP
GGSRKLSCAASGFTFSNYGMHWVRQAPEKGLEWVAYISSGSSTIYYADTV
KGRFTISRDNAKNTLFLQMTSLRSEDTAMYYCARRGLLLDYWGQGTTLTV
SSLEQKLISEEDLNSAVDHHHHHH.

Annotation of scFv Protein Domains with Reference to SEQ ID NO:27
AGIH sequence increases solubility (amino acids 1-4 of SEQ ID NO:27)
Vk variable region (amino acids 5-115 of SEQ ID NO:27)
Initial (6 aa) of light chain CH1 (amino acids 116-121 of SEQ ID NO:27)
(GGGGS) 3 (SEQ ID NO:26) linker (amino acids 122-136 of SEQ ID NO:27)
VH variable region (amino acids 137-252 of SEQ ID NO:27)
Myc tag (amino acids 253-268 SEQ ID NO:27)
His 6 tag (amino acids 269-274 of SEQ ID NO:27).
   Amino acid sequence of 3E10 di-scFv (D3IN)

Di-scFv 3E10 (D31N) is a di-single chain variable fragment including 2× the heavy chain and light chain variable regions of 3E10 and wherein the aspartic acid at position 31 of the heavy chain is mutated to an asparagine. The amino acid sequence for di-scFv 3E10 (D31N) is:

(SEQ ID NO: 28)
AGIHDIVLTQSPASLAVSLGQRATISCRASKSVSTSSYSYMHWYQQKPGQ
PPKLLIKYASYLESGVPARFSGSGSGTDFTLNIHPVEEEDAATYYCQHSR
EFPWTFGGGTKLEIKRADAAPGGGGSGGGGSGGGGSEVQLVESGGGLVKP
GGSRKLSCAASGFTFSNYGMHWVRQAPEKGLEWVAYISSGSSTIYYADTV
KGRFTISRDNAKNTLFLQMTSLRSEDTAMYYCARRGLLLDYWGQGTTLTV
SSASTKGPSVFPLAPLESSGSDIVLTQSPASLAVSLGQRATISCRASKSV
STSSYSYMHWYQQKPGQPPKLLIKYASYLESGVPARFSGSGSGTDFTLNI
HPVEEEDAATYYCQHSREFPWTFGGGTKLEIKRADAAPGGGGSGGGGSGG
GGSEVQLVESGGGLVKPGGSRKLSCAASGFTFSNYGMHWVRQAPEKGLEW
VAYISSGSSTIYYADTVKGRFTISRDNAKNTLFLQMTSLRSEDTAMYYCA
RRGLLLDYWGQGTTLTVSSLEQKLISEEDLNSAVDHHHHHH.

Annotation of di-scFv Protein Domains with Reference to SEQ ID NO:28
AGIH sequence increases solubility (amino acids 1-4 of SEQ ID NO:28)
Vk variable region (amino acids 5-115 of SEQ ID NO:28)
Initial (6 aa) of light chain CH1 (amino acids 116-121 of SEQ ID NO:28)
(GGGGS) 3 (SEQ ID NO:26) linker (amino acids 122-136 of SEQ ID NO:28)
VH variable region (amino acids 137-252 of SEQ ID NO:28)
Linker between Fv fragments consisting of human IgG CH1 initial 13 amino acids (amino acids 253-265 of SEQ ID NO:28)
Swivel sequence (amino acids 266-271 of SEQ ID NO:28)
Vk variable region (amino acids 272-382 of SEQ ID NO:28)
Initial (6 aa) of light chain CH1 (amino acids 383-388 of SEQ ID NO:28)
(GGGGS) 3 (SEQ ID NO:26) linker (amino acids 389-403 of SEQ ID NO:28)
VH variable region (amino acids 404-519 of SEQ ID NO:28)
Myc tag (amino acids 520-535 of SEQ ID NO:28)
His 6 tag (amino acids 536-541 of SEQ ID NO:28)

### Amino Acid Sequence for Tri-scFv

Tri-scFv 3E10 (D31N) is a tri-single chain variable fragment including 3× the heavy chain and light chain variable regions of 310E and wherein the aspartic acid at position 31 of the heavy chain is mutated to an asparagine. The amino acid sequence for tri-scFv 3E10 (D31N) is:

(SEQ ID NO: 29)
AGIHDIVLTQSPASLAVSLGQRATISCRASKSVSTSSYSYMHWYQQKPGQ
PPKLLIKYASYLESGVPARFSGSGSGTDFTLNIHPVEEEDAATYYCQHSR
EFPWTFGGGTKLEIKRADAAPGGGGSGGGGSGGGGSEVQLVESGGGLVKP
GGSRKLSCAASGFTFSNYGMHWVRQAPEKGLEWVAYISSGSSTIYYADTV
KGRFTISRDNAKNTLFLQMTSLRSEDTAMYYCARRGLLLDYWGQGTTLTV
SSASTKGPSVFPLAPLESSGSDIVLTQSPASLAVSLGQRATISCRASKSV
STSSYSYMHWYQQKPGQPPKLLIKYASYLESGVPARFSGSGSGTDFTLNI
HPVEEEDAATYYCQHSREFPWTFGGGTKLEIKRADAAPGGGGSGGGGSGG
GGSEVQLVESGGGLVKPGGSRKLSCAASGFTFSNYGMHWVRQAPEKGLEW
VAYISSGSSTIYYADTVKGRFTISRDNAKNTLFLQMTSLRSEDTAMYYCA
RRGLLLDYWGQGTTLTVSSASTKGPSVFPLAPLESSGSDIVLTQSPASLA
VSLGQRATISCRASKSVSTSSYSYMHWYQQKPGQPPKLLIKYASYLESGV
PARFSGSGSGTDFTLNIHPVEEEDAATYYCQHSREFPWTFGGGTKLEIKR
ADAAPGGGGSGGGGSGGGGSEVQLVESGGGLVKPGGSRKLSCAASGFTFS
NYGMHWVRQAPEKGLEWVAYISSGSSTIYYADTVKGRFTISRDNAKNTLF
LQMTSLRSEDTAMYYCARRGLLLDYWGQGTTLTVSSLEQKLISEEDLNSA
VDHHHHHH.

Annotation of tri-scFv Protein Domains with Reference to SEQ ID NO:29
AGIH sequence increases solubility (amino acids 1-4 of SEQ ID NO:29)
Vk variable region (amino acids 5-115 of SEQ ID NO:29)
Initial (6 aa) of light chain CH1 (amino acids 116-121 of SEQ ID NO:29)
(GGGGS) 3 (SEQ ID NO:26) linker (amino acids 122-136 of SEQ ID NO:29)
V_{H} variable region (amino acids 137-252 of SEQ ID NO:29)
Linker between Fv fragments consisting of human IgG CH1 initial 13 amino acids (amino acids 253-265 of SEQ ID NO:29)
Swivel sequence (amino acids 266-271 of SEQ ID NO:29)
Vk variable region (amino acids 272-382 of SEQ ID NO:29)
Initial (6 aa) of light chain CH1 (amino acids 383-388 of SEQ ID NO:29)
(GGGGS) 3 (SEQ ID NO:26) linker (amino acids 389-403 of SEQ ID NO:29
V_{H} variable region (amino acids 404-519 of SEQ ID NO:29)
Linker between Fv fragments consisting of human IgG CH1 initial 13 amino acids (amino acids 520-532 of SEQ ID NO:29)
Swivel sequence (amino acids 533-538 of SEQ ID NO:29)
Vk variable region (amino acids 539-649 of SEQ ID NO:29)
Initial (6 aa) of light chain CH1 (amino acids 650-655 of SEQ ID NO:29)
(GGGGS) 3 (SEQ ID NO:26) linker (amino acids 656-670 of SEQ ID NO:29)
V_{H} variable region (amino acids 671-786 of SEQ ID NO:29)
Myc tag (amino acids 787-802 of SEQ ID NO:29)
His 6 tag (amino acids 803-808 of SEQ ID NO:29)

WO 2016/033321 and Noble, et al., Cancer Research, 75 (11): 2285-2291 (2015), show that di-scFv and tri-scFv have some improved and additional activities compared to their monovalent counterpart. The subsequences corresponding to the different domains of each of the exemplary fusion proteins are also provided above. One of skill in the art will appreciate that the exemplary fusion proteins, or domains thereof, can be utilized to construct fusion proteins discussed in more detail above. For example, in some embodiments, the di-scFv includes a first scFv including a Vk variable region (e.g., amino acids 5-115 of SEQ ID NO: 28, or a functional variant or fragment thereof), linked to a VH variable domain (e.g., amino acids 137-252 of SEQ ID NO:28, or a functional variant or fragment thereof), linked to a second scFv including a Vk variable region (e.g., amino acids 272-382 of SEQ ID NO: 28, or a functional variant or fragment thereof), linked to a VH variable domain (e.g., amino acids 404-519 of SEQ ID NO:28, or a functional variant or fragment thereof). In some embodiments, a tri-scFv includes a di-scFv linked to a third scFv domain including a Vk variable region (e.g., amino acids 539-649 of SEQ ID NO:29, or a functional variant or fragment thereof), linked to a VH variable domain (e.g., amino acids 671-786 of SEQ ID NO: 29, or a functional variant or fragment thereof).

The Vk variable regions can be linked to VH variable domains by, for example, a linker (e.g., (GGGGS) 3 (SEQ ID NO:26), alone or in combination with a (6 aa) of light chain CH1 (amino acids 116-121 of SEQ ID NO:28). Other suitable linkers are discussed above and known in the art. scFv can be linked by a linker (e.g., human IgG CH1 initial 13 amino acids (253-265) of SEQ ID NO:28), alone or in combination with a swivel sequence (e.g., amino acids 266-271 of SEQ ID NO:28). Other suitable linkers are discussed above and known in the art.

Therefore, a di-scFv can include amino acids 5-519 of SEQ ID NO:28. A tri-scFv can include amino acids 5-786 of SEQ ID NO:29. In some embodiments, the fusion proteins include additional domains. For example, in some embodiments, the fusion proteins include sequences that enhance solubility (e.g., amino acids 1~4 of SEQ ID NO:28). Therefore, in some embodiments, a di-scFv can include amino acids 1-519 of SEQ ID NO:28. A tri-scFv can include amino acids 1-786 of SEQ ID NO:29. In some embodiments that fusion proteins include one or more domains that enhance purification, isolation, capture, identification, separation, etc., of the fusion protein. Exemplary domains include, for example, Myc tag (e.g., amino acids 520-535 of SEQ ID NO:28) and/or a His tag (e.g., amino acids 536-541 of SEQ ID NO:28). Therefore, in some embodiments, a di-scFv can include the amino acid sequence of SEQ ID NO:28. A tri-scFv can include the amino acid sequence of SEQ ID NO: 29. Other substitutable domains and additional domains are discussed in more detail above.

### B. Immune Checkpoint Modulators

The methods typically include administering an immune checkpoint modulator. Immune checkpoints can be stimulatory or inhibitory, and tumors can use these checkpoints to protect themselves from immune system attacks. Currently approved checkpoint therapies block inhibitory checkpoint receptors, but investigations into therapies that activate stimulatory checkpoints are also underway. Thus, the immune checkpoint modulator can be one that blocks an inhibitory checkpoint, or activates a stimulatory checkpoint. Typically, the immune checkpoint modulator is one that induces or otherwise activates or increases an immune response against target cells for example cancer cells or infected cells. Accordingly, in some embodiments, the immune checkpoint modulator can be a chimeric antigen receptor (CAR) directed cell such as a CAR-T cell. In another embodiment, the immune checkpoint modulator can be an oncolytic virus.

In preferred embodiments, the immune checkpoint modulator blocks an inhibitory checkpoint. Blockade of negative feedback signaling to immune cells thus results in an enhanced immune response against tumors. Thus, in some embodiments the immune checkpoint modulator is administered to the subject in an effective amount to block an inhibitory checkpoint. Exemplary compounds are those that block or otherwise inhibit, for example, PD-1, PD-L1, or CTLA4.

1. PD-1 antagonists

In some embodiments, the active agents are PD-1 antagonists. Activation of T cells normally depends on an antigen-specific signal following contact of the T cell receptor (TCR) with an antigenic peptide presented via the major histocompatibility complex (MHC) while the extent of this reaction is controlled by positive and negative antigen-independent signals emanating from a variety of co-stimulatory molecules. The latter are commonly members of the CD28/B7 family. Conversely, Programmed Death-1 (PD-1) is a member of the CD28 family of receptors that delivers a negative immune response when induced on T cells. Contact between PD-1 and one of its ligands (B7-H1 or B7-DC) induces an inhibitory response that decreases T cell multiplication and/or the strength and/or duration of a T cell response. Suitable PD-1 antagonists are described in U.S. Pat. Nos. 8,114,845, 8,609,089, and 8,709,416, and include compounds or agents that either bind to and block a ligand of PD-1 to interfere with or inhibit the binding of the ligand to the PD-1 receptor, or bind directly to and block the PD-1 receptor without inducing inhibitory signal transduction through the PD-1 receptor.

In some embodiments, the PD-1 receptor antagonist binds directly to the PD-1 receptor without triggering inhibitory signal transduction and also binds to a ligand of the PD-1 receptor to reduce or inhibit the ligand from triggering signal transduction through the PD-1 receptor. By reducing the number and/or amount of ligands that bind to PD-1 receptor and trigger the transduction of an inhibitory signal, fewer cells are attenuated by the negative signal delivered by PD-1 signal transduction and a more robust immune response can be achieved.

It is believed that PD-1 signaling is driven by binding to a PD-1 ligand (such as B7-H1 or B7-DC) in close proximity to a peptide antigen presented by major histocompatibility complex (MHC) (see, for example, Freeman, Proc. Natl. Acad. Sci. U. S. A, 105:10275-10276 (2008)). Therefore, proteins, antibodies or small molecules that prevent co-ligation of PD-1 and TCR on the T cell membrane are also useful PD-1 antagonists.

In preferred embodiments, the PD-1 receptor antagonists are small molecule antagonists or antibodies that reduce or interfere with PD-1 receptor signal transduction by binding to ligands of PD-1 or to PD-1 itself, especially where co-ligation of PD-1 with TCR does not follow such binding, thereby not triggering inhibitory signal transduction through the PD-1 receptor.

Other PD-1 antagonists include antibodies that bind to PD-1 or ligands of PD-1 such as PD-L1 (also known as B7-H1) and PD-L2 (also known as B7-DC), and other antibodies.

Suitable anti-PD-1 antibodies include, but are not limited to, those described in the following publications:
PCT/IL03/00425 (Hardy et al., WO/2003/099196)
PCT/JP2006/309606 (Korman et al., WO/2006/121168)
PCT/US2008/008925 (Li et al., WO/2009/014708)
PCT/JP03/08420 (Honjo et al., WO/2004/004771)
PCT/JP04/00549 (Honjo et al., WO/2004/072286)
PCT/IB2003/006304 (Collins et al., WO/2004/056875)
PCT/US2007/088851 (Ahmed et al., WO/2008/083174)
PCT/US2006/026046 (Korman et al., WO/2007/005874)
PCT/US2008/084923 (Terrett et al., WO/2009/073533)

Berger et al., Clin. Cancer Res., 14:30443051 (2008).

A specific example of an anti-PD-1 antibody is MDX-1106 (see Kosak, U.S. Pat. No. 20,070,166281 (pub. 19 Jul. 2007) at par. 42), a human anti-PD-1 antibody, preferably administered at a dose of 3 mg/kg.

Exemplary anti-B7-H1 antibodies include, but are not limited to, those described in the following publications:
PCT/US06/022423 (WO/2006/133396, pub. 14 Dec. 2006)
PCT/US07/088851 (WO/2008/083174, pub. 10 Jul. 2008)
US 2006/0110383 (pub. 25 May 2006)

A specific example of an anti-B7-H1 antibody is MDX-1105 (WO/2007/005874, published 11 Jan. 2007)), a human anti-B7-H1 antibody.

For anti-B7-DC antibodies see 7,411,051, 7,052,694, 7,390,888, and U.S. Published Application No. 2006/0099203.

The antibody can be a bi-specific antibody that includes an antibody that binds to the PD-1 receptor bridged to an antibody that binds to a ligand of PD-1, such as B7-H1. In some embodiments, the PD-1 binding portion reduces or inhibits signal transduction through the PD-1 receptor.

Other exemplary PD-1 receptor antagonists include, but are not limited to B7-DC polypeptides, including homologs and variants of these, as well as active fragments of any of the foregoing, and fusion proteins that incorporate any of these. In a preferred embodiment, the fusion protein includes the soluble portion of B7-DC coupled to the Fc portion of an antibody, such as human IgG, and does not incorporate all or part of the transmembrane portion of human B7-DC.

The PD-1 antagonist can also be a fragment of a mammalian B7-H1, preferably from mouse or primate, preferably human, wherein the fragment binds to and blocks PD-1 but does not result in inhibitory signal transduction through PD-1. The fragments can also be part of a fusion protein, for example an Ig fusion protein.

Other useful polypeptides PD-1 antagonists include those that bind to the ligands of the PD-1 receptor. These include the PD-1 receptor protein, or soluble fragments thereof, which can bind to the PD-1 ligands, such as B7-H1 or B7-DC, and prevent binding to the endogenous PD-1 receptor, thereby preventing inhibitory signal transduction. B7-H1 has also been shown to bind the protein B7.1 (Butte et al., Immunity, Vol. 27, pp. 111-122, (2007)). Such fragments also include the soluble ECD portion of the PD-1 protein that includes mutations, such as the A99L mutation, that increases binding to the natural ligands (Molnar et al., PNAS, 105:10483-10488 (2008)). B7-1 or soluble fragments thereof, which can bind to the B7-H1 ligand and prevent binding to the endogenous PD-1 receptor, thereby preventing inhibitory signal transduction, are also useful.

PD-1 and B7-H1 anti-sense nucleic acids, both DNA and RNA, as well as siRNA molecules can also be PD-1 antagonists. Such anti-sense molecules prevent expression of PD-1 on T cells as well as production of T cell ligands, such as B7-H1, PD-L1 and/or PD-L2. For example, siRNA (for example, of about 21 nucleotides in length, which is specific for the gene encoding PD-1, or encoding a PD-1 ligand, and which oligonucleotides can be readily purchased commercially) complexed with carriers, such as polyethyleneimine (see Cubillos-Ruiz et al., J. Clin. Invest. 119 (8): 2231-2244 (2009), are readily taken up by cells that express PD-1 as well as ligands of PD-1 and reduce expression of these receptors and ligands to achieve a decrease in inhibitory signal transduction in T cells, thereby activating T cells.

Exemplary PD-1 inhibitors include, but are not limited to,
Pembrolizumab (formerly MK-3475 or lambrolizumab, Keytruda) was developed by Merck and first approved by the Food and Drug Administration in 2014 for the treatment of melanoma.
Nivolumab (Opdivo) was developed by Bristol-Myers Squibb and first approved by the FDA in 2014 for the treatment of melanoma.
pidilizumab, by CureTech
AMP-224, by GlaxoSmithKline and MedImmune
AMP-514, by GlaxoSmithKline and MedImmune
PDR001, by Novartis
cemiplimab, by Regeneron and Sanofi

Exemplary PD-L1 inhibitors include, but are not limited to,
Atezolizumab (Tecentriq) is a fully humanised IgG1 (immunoglobulin 1 antibody developed by Roche Genentech. In 2016, the FDA approved atezolizumab for urothelial carcinoma and non-small cell lung cancer.
Avelumab (Bavencio) is a fully human IgG1 antibody developed by Merck Serono and Pfizer. Avelumab is FDA approved for the treatment of metastatic merkel-cell carcinoma. It failed phase III clinical trials for gastric cancer.
Durvalumab (Imfinzi) is a fully human IgG1 antibody developed by AstraZeneca. Durvalumab is FDA approved for the treatment of urothelial carcinoma and unresectable non-small cell lung cancer after chemoradiation.
BMS-936559, by Bristol-Myers Squibb
CK-301, by Checkpoint Therapeutics

See, e.g., Iwai, et al., Journal of Biomedical Science, (2017) 24:26, DOI 10.1186/s12929-017-0329-9.

2. CTLA4 antagonists

Other molecules useful in mediating the effects of T cells in an immune response are also contemplated as active agents. For example, in some embodiments, the molecule is an agent binds to an immune response mediating molecule that is not PD-1. In a preferred embodiment, the molecule is an antagonist of CTLA4, for example an antagonistic anti-CTLA4 antibody. An example of an anti-CTLA4 antibody is described in PCT/US2006/043690 (Fischkoff et al., WO/2007/056539).

Dosages for anti-PD-1, anti-B7-H1, and anti-CTLA4 antibody, are known in the art and can be in the range of 0.1 to 100 mg/kg, with shorter ranges of 1 to 50 mg/kg preferred and ranges of 10 to 20 mg/kg being more preferred. An appropriate dose for a human subject is between 5 and 15 mg/kg, with 10 mg/kg of antibody (for example, human anti-PD-1 antibody, like MDX-1106) most preferred.

Specific examples of CTLA antagonists include Ipilimumab, also known as MDX-010 or MDX-101, a human anti-CTLA4 antibody, preferably administered at a dose of about 10 mg/kg, and Tremelimumab a human anti-CTLA4 antibody, preferably administered at a dose of about 15 mg/kg. See also Sammartino, et al., Clinical Kidney Journal, 3 (2): 135-137 (2010), published online December 2009.

In other embodiments, the antagonist is a small molecule. A series of small organic compounds have been shown to bind to the B7-1 ligand to prevent binding to CTLA4 (see Erbe et al., J. Biol. Chem., 277:7363-7368 (2002). Such small organics could be administered alone or together with an anti-CTLA4 antibody to reduce inhibitory signal transduction of T cells.

### 3. Chimeric Antigen Receptor Directed Cells

The modulator can be a chimeric antigen receptor directed cell. The term “Chimeric Antigen Receptor” or alternatively a “CAR” refers to a set of polypeptides, typically two in the simplest embodiments, which when in an immune effector cell, provides the cell with specificity for a cancer cell, and with intracellular signal generation. In some embodiments, a CARincludes at least an antigen binding domain such as an extracellular binding domain, a transmembrane domain and a cytoplasmic signaling domain (also referred to as “an intracellular signaling domain”) including a functional signaling domain derived from a stimulatory molecule and/or costimulatory molecule as defined below. In one embodiment, the stimulatory molecule is a zeta chain (“zeta stimulatory domain”) associated with a T cell receptor complex. In one embodiment, the cytoplasmic signaling domain further includes one or more functional signaling domains derived from at least one costimulatory molecule (e.g., 4-1BB (i.e., CD137), CD27 and/or CD28). In some embodiments, the CAR includes a chimeric fusion protein including an extracellular antigen binding domain, a transmembrane domain and an intracellular signaling domain including a functional signaling domain derived from a stimulatory molecule. In various embodiments, CARs are fusion proteins of single-chain variable fragments (scFv) fused to a CD3-zeta transmembrane domain. However, other intracellular signaling domains such as CD28, 41-BB and Ox40 may be used in various combinations to give the desired intracellular signal. In some embodiments, CARs disclosed herein include an extracellular binding domain.

The term “antigen binding domain” is used in the context of the present disclosure to refer to the portion of the CAR that specifically recognizes and binds to the antigen of interest. The “antigen binding domain” may be derived from a binding protein disclosed herein such as an antibody or fragment thereof. In some embodiments, the “binding domain” is a single-chain variable fragment (scFv). In certain embodiments, the “binding domain” includes the complementarity determining regions of a binding protein disclosed herein. In this embodiment, the CAR directed cell can represent the combination of a cell-penetrating antibody (assuming it penetrates a cancer cell) that induces or increase DNA damage or reduces or impairs DNA damage repair, or a combination thereof and an immune checkpoint modulator that induces, increases, or enhances an immune response. For example, the binding domain can represent the cell-penetrating antibody and the modified T-cell can represent the immune cell modulator. In another example, a CAR-directed cell disclosed herein is administered with a cell-penetrating antibody disclosed herein.

The terms “zeta” or “CD3-zeta” are used herein to define the protein provided as GenBan Acc. No. BAG36664.1, or the equivalent residues from a non-human species and a “zeta stimulatory domain” or alternatively a “CD3-zeta stimulatory domain” is defined as the amino acid residues from the cytoplasmic domain of the zeta chain, or functional derivatives thereof, that are sufficient to functionally transmit an initial signal necessary for T cell activation.

The term “immune effector cell,” is used herein to refer to a cell that is involved in an immune response (e.g. promotion of an immune effector response). Examples of immune effector cells include T cells, e.g., alpha/beta T cells and gamma/delta T cells, B cells, natural killer (NK) cells, natural killer T (NKT) cells, mast cells, and myeloic-derived phagocytes. In some embodiments, the immune effector cell(s) is allogenic. In some embodiments, the immune effector cell(s) is autologous. In some embodiments, the immune checkpoint modulator is a CAR directed T cell (CAR-T cell). Exemplary CAR-T cells include Axicabtagene ciloleucel (KTE-C19, Axi-cel), Tisagenlecleucel, Lisocabtagene Maraleucel (liso-cel; JCAR017).

Immune effector cells such as T cells may be activated and expanded generally using methods previously described, such as for example, as described in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041. As a general example, a population of immune effector cells e.g., T regulatory cell depleted cells, may be expanded by contact with a surface having attached thereto an agent that stimulates a CD3 complex associated signal and a ligand that stimulates a costimulatory molecule on the surface of the T cells.

### 4. Oncolytic Virus

The modulator can be an oncolytic virus. The term “oncolytic virus” is used in the context of the present disclosure to refer to viruses that are able to infect and reduce growth of cancer cells. For example, oncolytic viruses can inhibit cell proliferation. In some embodiments, oncolytic viruses can kill cancer cells. In some embodiments, the oncolytic virus preferentially infects and inhibits growth of cancer cells compared with corresponding normal cells. In another embodiment, the oncolytic virus preferentially replicates in and inhibits growth of cancer cells compared with corresponding normal cells.

In some embodiments, the oncolytic virus is able to naturally infect and reduce growth of cancer cells. Examples of such viruses include Newcastle disease virus, vesicular stomatitis, myxoma, reovirus, sindbis, measles and coxsackievirus. Oncolytic viruses able to naturally infect and reduce growth of cancer cells generally target cancer cells by exploiting the cellular aberrations that occur in these cells. For example, oncolytic viruses may exploit surface attachment receptors, activated oncogenes such as Ras, Akt, p53 and/or interferon (IFN) pathway defects.

In another embodiment, oncolytic viruses encompassed by the present disclosure are engineered to infect and reduce growth of cancer cells. Exemplary viruses suitable for such engineering include oncolytic DNA viruses, such as adenovirus, herpes simplex virus (HSV) and Vaccinia virus; and oncolytic RNA viruses such as Lentivirus, Reovirus, Coxsackievirus, Seneca Valley Virus, Poliovirus, Measles virus, Newcastle disease virus, Vesicular stomatitis virus (VSV) and parvovirus such as rodent protoparvoviruses H-1PV. In some embodiments, the oncolytic virus includes a backbone of an above referenced virus.

In some embodiments, tumor specificity of an oncolytic virus can be engineered to mutate or delete gene(s) required for survival of the virus in normal cells but expendable in cancer cells. For example, the oncolytic virus can be engineered by mutating or deleting a gene that encodes thymidine kinase, an enzyme needed for nucleic acid metabolism. In this example, viruses are dependent on cellular thymidine kinase expression, which is high in proliferating cancer cells but repressed in normal cells. In another example, the oncolytic virus is engineered to include a capsid protein that binds a tumor specific cell surface molecule. In some embodiments, the capsid protein is a fibre, a penton or hexon protein. In another example, the oncolytic virus is engineered to include a tumor specific cell surface molecule for transductionally targeting a cancer cell. Exemplary tumor specific cell surface molecules can include an integrin, an EGF receptor family member, a proteoglycan, a disialoganglioside, B7-H3, CA-125, EpCAM, ICAM-1, DAF, A21, integrin-a2ß1, vascular endothelial growth factor receptor 1, vascular endothelial growth factor receptor 2, CEA, a tumour associated glycoprotein, CD19, CD20, CD22, CD30, CD33, CD40, CD44, CD52, CD74, CD152, CD155, MUC1, a tumour necrosis factor receptor, an insulin-like growth factor receptor, folate receptor a, transmembrane glycoprotein NMB, a C-C chemokine receptor, PSMA, RON-receptor, and cytotoxic T-lymphocyte antigen 4.

The oncolytic virus can be replication-competent. In some embodiments, the oncolytic viruses selectively replicate in cancer cells when compared with corresponding normal cells.

Conditional replication can be achieved by, for example, the insertion of a tumor-specific promoter driving the expression of a critical gene(s). Such promoters can be identified based on differences in gene expression between tumor and corresponding surrounding tissue. Exemplary native promoters include AFP, CCKAR, CEA, erbB2, Cerb2, COX2, CXCR4, E2F1, HE4, LP, MUC1, PSA, Survivin, TRP1, STAT3, hTERT and Tyr. Exemplary composite promoters include AFP/hAFP, SV40/AFP, CEA/CEA, PSA/PSA, SV40/Tyr and Tyr/Tyr.

Various viruses may be engineered as outlined in the above referenced examples. The oncolytic virus can be, for example, a modified HSV, Lentivirus, Baculovirus, Retrovirus, Adenovirus (AdV), Adeno-associated virus (AAV) or a recombinant form such as recombinant adeno-associated virus (rAAV) or a derivative thereof such as a self-complementary AAV (scAAV) or non-integrating AV. The oncolytic virus can be a modified HSVThe oncolytic virus can be a modified lentivirus. Other exemplary viruses include vaccina virus, vesicular stomatitis virus (VSV), measles virus and maraba virus.

In other examples, the oncolytic virus may be one of various AV or AAV serotypes. In some embodiments, the oncolytic virus is serotype 1. In another example, the oncolytic virus is serotype 2. In other examples, the oncolytic virus is serotype 3, 4, 7, 8, 9, 10, 11, 12 or 13. In another example, the oncolytic virus is serotype 5. In another example, the oncolytic virus is serotype 6.

Exemplary oncolytic viruses include T-Vec (HSV-1; Amgen), JX-594 (Vaccina; Sillajen), JX-594 (AdV; Cold Genesys), Reolysin (Reovirus; Oncolytics Biotech). Other examples of oncolytic viruses are disclosed in WO 2003/080083, WO 2005/086922, WO 2007/088229, WO 2008/110579, WO 2010/108931, WO 2010/128182, WO 2013/112942, WO 2013/116778, WO 2014/204814, WO 2015/077624 and WO 2015/166082, WO 2015/089280.

### 5. Other Immune Checkpoint Modulators

Other immune checkpoint targets include, but are not limited to, ICOS, OX40, GITR, 4-1BB, CD40, CD27-CD70, LAG3, TIM-3, TIGIT, VISTA, B7-H3, KIR, PARP, and others, and are being targeting for cancer treatment alone and in combination with anti-PD-1, anti-PD-L1, and anti-CTLA compounds. See, for example, Iwai, et al., Journal of Biomedical Science. 24 (1): 26. doi: 10.1186/s12929-017-0329-9; Donini, et al., J Thorac Dis. 2018 May; 10 (Suppl 13): S1581-S1601. doi: 10.21037/jtd.2018.02.79. Thus, in some embodiments, a cell-penetrating antibody is administered in combination with a compound that targets ICOS, OX40, GITR, 4-1BB, CD40, CD27-CD70, LAG3, TIM-3, TIGIT, VISTA, B7-H3, KIR, or PARP, or a combination thereof, alone or in combination with a compound that target PD-1, PD-L1, and/or CTLA. In another embodiment, the immune checkpoint modulator is an antibody disclosed in WO 2016/013870.

### C. Formulations

### 1. Pharmaceutical Compositions

Any of the disclosed compositions can be formulated in a pharmaceutical composition with, for example, a pharmaceutically acceptable carrier. The compositions can be in solution, emulsions, or suspension (for example, incorporated into microparticles, liposomes, or cells). Typically, an appropriate amount of a pharmaceutically-acceptable salt is used in the formulation to render the formulation isotonic. As used herein, the term “pharmaceutically acceptable carrier” encompasses any of the standard pharmaceutical carriers, such as a phosphate buffered saline solution, water and emulsions such as an oil/water or water/oil emulsion, various types of wetting agents, and others disclosed herein and/or known in the art. Examples of pharmaceutically-acceptable carriers include, but are not limited to, saline, Ringer's solution and dextrose solution. The pH of the solution is preferably from about 5 to about 8, and more preferably from about 7 to about 7.5. Pharmaceutical compositions may include carriers, thickeners, diluents, buffers, preservatives, and surface active agents. Further carriers include sustained release preparations such as semi-permeable matrices of solid hydrophobic polymers containing the antibody, which matrices are in the form of shaped particles, e.g., films, liposomes or microparticles. It will be apparent to those persons skilled in the art that certain carriers may be more preferable depending upon, for instance, the route of administration and concentration of composition being administered. Pharmaceutical compositions may also include one or more active ingredients such as antimicrobial agents, anti-inflammatory agents, and anesthetics.

In exemplary preferred embodiments, the compositions can be formulated in a pharmaceutical composition that is suitable for administration by parenteral route, especially injectable or infusable preparations, those forms allowing the immediate release or delayed and controlled release of the active ingredient.

The compositions can be administered systemically. Preferably, the composition is delivered in manner such that the active agent contacts target tissues, and does not or only minimally contacts tissue that could cause a toxic or adverse event. In some embodiments, the composition is delivered locally to a tumor to the tumor's microenvironment. For example, in a particular embodiment, one or more of the compositions are delivered by intratumoral injection.

Drugs can be formulated for immediate release, extended release, or modified release. A delayed release dosage form is one that releases a drug (or drugs) at a time other than promptly after administration. An extended release dosage form is one that allows at least a twofold reduction in dosing frequency as compared to that drug presented as a conventional dosage form (e.g. as a solution or prompt drug-releasing, conventional solid dosage form). A modified release dosage form is one for which the drug release characteristics of time course and/or location are chosen to accomplish therapeutic or convenience objectives not offered by conventional dosage forms such as solutions, ointments, or promptly dissolving dosage forms. Delayed release and extended release dosage forms and their combinations are types of modified release dosage forms.

Formulations are prepared using a pharmaceutically acceptable “carrier” composed of materials that are considered safe and effective and may be administered to an individual without causing undesirable biological side effects or unwanted interactions. The “carrier” is all components present in the pharmaceutical formulation other than the active ingredient or ingredients. The term “carrier” includes, but is not limited to, diluents, binders, lubricants, desintegrators, fillers, and coating compositions.

The cell-penetrating binding protein, such as an antibody, and/or the immune checkpoint modulator can be administered to a subject with or without the aid of a delivery vehicle. Appropriate delivery vehicles for the compounds are known in the art and can be selected to suit the particular active agent. For example, in some embodiments, the active agent(s) is incorporated into or encapsulated by, or bound to, a nanoparticle, microparticle, micelle, synthetic lipoprotein particle, or carbon nanotube. For example, the compositions can be incorporated into a vehicle such as polymeric particles which provide controlled release of the active agent(s). In some embodiments, release of the drug(s) is controlled by diffusion of the active agent(s) out of the particles and/or degradation of the polymeric particles by hydrolysis and/or enzymatic degradation.

Suitable polymers include ethylcellulose and other natural or synthetic cellulose derivatives. Polymers which are slowly soluble and form a gel in an aqueous environment, such as hydroxypropyl methylcellulose or polyethylene oxide, may also be suitable as materials for drug containing microparticles or particles. Other polymers include, but are not limited to, polyanhydrides, poly(ester anhydrides), polyhydroxy acids, such as polylactide (PLA), polyglycolide (PGA), poly(lactide-co-glycolide) (PLGA), poly-3-hydroxybut rate (PHB) and copolymers thereof, poly-4-hydroxybutyrate (P4HB) and copolymers thereof, polycaprolactone and copolymers thereof, and combinations thereof. In some embodiments, both agents are incorporated into the same particles and are formulated for release at different times and/or over different time periods. For example, in some embodiments, one of the agents is released entirely from the particles before release of the second agent begins. In other embodiments, release of the first agent begins followed by release of the second agent before the all of the first agent is released. In still other embodiments, both agents are released at the same time over the same period of time or over different periods of time. Two or more active agents can also be packaged in separate particles of the same or different polymeric composition.

Agents and pharmaceutical compositions thereof can be administered in an aqueous solution, by parenteral injection. The formulation may also be in the form of a suspension or emulsion. In general, pharmaceutical compositions are provided including effective amounts of the active agent(s) and optionally include pharmaceutically acceptable diluents, preservatives, solubilizers, emulsifiers, adjuvants and/or carriers. Such compositions include diluents sterile water, buffered saline of various buffer content (e.g., Tris-HCl, acetate, phosphate), pH and ionic strength; and optionally, additives such as detergents and solubilizing agents (e.g., TWEEN® 20, TWEEN® 80 also referred to as polysorbate 20 or 80), anti-oxidants (e.g., ascorbic acid, sodium metabisulfite), and preservatives (e.g., Thimersol, benzyl alcohol) and bulking substances (e.g., lactose, mannitol). Examples of non-aqueous solvents or vehicles are propylene glycol, polyethylene glycol, vegetable oils, such as olive oil and corn oil, gelatin, and injectable organic esters such as ethyl oleate. The formulations may be lyophilized and redissolved/resuspended immediately before use. The formulation may be sterilized by, for example, filtration through a bacteria retaining filter, by incorporating sterilizing agents into the compositions, by irradiating the compositions, or by heating the compositions.

To aid dissolution of antibody fragments or fusion proteins into the aqueous environment a surfactant might be added as a wetting agent. Surfactants may include anionic detergents such as sodium lauryl sulfate, dioctyl sodium sulfosuccinate and dioctyl sodium sulfonate. Cationic detergents might be used and could include benzalkonium chloride or benzethomium chloride. The list of potential nonionic detergents that could be included in the formulation as surfactants are lauromacrogol 400, polyoxyl 40 stearate, polyoxyethylene hydrogenated castor oil 10, 50 and 60, glycerol monostearate, polysorbate 20, 40, 60, 65 and 80, sucrose fatty acid ester, methyl cellulose and carboxymethyl cellulose. These surfactants could be present in the formulation of the protein or derivative either alone or as a mixture in different ratios. Additives which potentially enhance uptake of peptides are for instance the fatty acids oleic acid, linoleic acid and linolenic acid.

In some embodiments, particularly enteral, transdermal, and transmucosal formulations, the compositions include excipients that protect the antibody and/or other active agents from degradation.

### a. Exemplary Formulations for Parenteral Administration

The compositions can be administered in an aqueous solution, by parenteral injection. The formulation may also be in the form of a suspension or emulsion. In general, pharmaceutical compositions are provided including effective amounts of the active agent and optionally include pharmaceutically acceptable diluents, preservatives, solubilizers, emulsifiers, adjuvants and/or carriers. Such compositions include diluents sterile water, buffered saline of various buffer content (e.g., Tris-HCl, acetate, phosphate), pH and ionic strength; and optionally, additives such as detergents and solubilizing agents (e.g., TWEEN® 20, TWEEN® 80 also referred to as polysorbate 20 or 80), anti-oxidants (e.g., ascorbic acid, sodium metabisulfite), and preservatives (e.g., Thimersol, benzyl alcohol) and bulking substances (e.g., lactose, mannitol). Examples of non-aqueous solvents or vehicles are propylene glycol, polyethylene glycol, vegetable oils, such as olive oil and corn oil, gelatin, and injectable organic esters such as ethyl oleate. The formulations may be lyophilized and redissolved/resuspended immediately before use. The formulation may be sterilized by, for example, filtration through a bacteria retaining filter, by incorporating sterilizing agents into the compositions, by irradiating the compositions, or by heating the compositions.

### b. Exemplary Oral Formulations

Oral formulations may be in the form of chewing gum, gel strips, tablets or lozenges. Encapsulating substances for the preparation of enteric-coated oral formulations include cellulose acetate phthalate, polyvinyl acetate phthalate, hydroxypropyl methylcellulose phthalate and methacrylic acid ester copolymers. Solid oral formulations such as capsules or tablets are preferred. Elixirs and syrups also are well known oral formulations. The components of aerosol formulations include solubilized active ingredients, antioxidants, solvent blends and propellants for solution formulations, and micronized and suspended active ingredients, dispersing agents and propellants for suspension formulations. The oral, aerosol and nasal formulations of the invention can be distinguished from injectable preparations of the prior art because such formulations may be nonaseptic, whereas injectable preparations must be aseptic.

### c. Exemplary Formulations for Topical Administration

The active agent can be applied topically. Topical administration can include application to the lungs, nasal, oral (sublingual, buccal), vaginal, or rectal mucosa.

Compositions can be delivered to the lungs while inhaling and traverse across the lung epithelial lining to the blood stream when delivered either as an aerosol or spray dried particles having an aerodynamic diameter of less than about 5 microns.

A wide range of mechanical devices designed for pulmonary delivery of therapeutic products can be used, including but not limited to nebulizers, metered dose inhalers, and powder inhalers, all of which are familiar to those skilled in the art. Some specific examples of commercially available devices are the Ultravent® nebulizer (Mallinckrodt Inc., St. Louis, Mo.); the Acorn® II nebulizer (Marquest Medical Products, Englewood, Colo.); the Ventolin® metered dose inhaler (Glaxo Inc., Research Triangle Park, N.C.); and the Spinhaler® powder inhaler (Fisons Corp., Bedford, Mass.). Nektar, Alkermes and Mannkind all have inhalable insulin powder preparations approved or in clinical trials where the technology could be applied to the formulations described herein.

Formulations for administration to the mucosa will typically be spray dried drug particles, which may be incorporated into a tablet, gel, capsule, suspension or emulsion. Standard pharmaceutical excipients are available from any formulator.

Transdermal formulations may also be prepared. These will typically be ointments, lotions, sprays, or patches, all of which can be prepared using standard technology. Transdermal formulations can include penetration enhancers.

### 2. Effective Amounts, Dosage, and Methods of Administration

In some embodiments, the pharmaceutical composition is a unit dosage containing the cell-penetrating binding protein, such as an antibody, the immune checkpoint modulator, or a combination thereof in a pharmaceutically acceptable excipient, wherein the cell-penetrating binding protein, such as an antibody, is present in an amount effective to induce DNA damage and/or impair DNA repair in a cancer or infected cell, the immune checkpoint modulator is in an active amount to induce or increase an immune response against the cancer or infected cell, or a combination thereof. In some embodiments, the pharmaceutical compositions can include one or more additional active agents. Therefore, in some embodiments, the pharmaceutical composition includes two, three, or more active agents. The precise dosage will vary according to a variety of factors such as subject-dependent variables (e.g., age, immune system health, clinical symptoms etc.). Exemplary dosages, symptoms, pharmacologic, and physiologic effects are discussed in more detail below. For example, effective dosages and schedules for administering the compositions may be determined empirically, and making such determinations is within the skill in the art. The dosage ranges for the administration of the compositions are those large enough to impair DNA repair in target cells and/or sensitize the target cells to radiotherapy and/or chemotherapy. The dosage should not be so large as to cause adverse side effects, such as unwanted cross-reactions, anaphylactic reactions, and the like. Generally, the dosage will vary with the age, condition, and sex of the patient, route of administration, whether other drugs are included in the regimen, and the type, stage, and location of the cancer or infection to be treated. The dosage can be adjusted by the individual physician in the event of any counter-indications. Dosage can vary, and can be administered in one or more dose administrations daily, for one or several days. Guidance can be found in the literature for appropriate dosages for given classes of pharmaceutical products. A typical daily dosage of binding protein, such as an antibody, might range from about 1 μg/kg to up to 200 mg/kg of body weight or more per day, depending on the factors mentioned above.

The timing of the administration of the compositions will depend on the formulation and/or route of administration used. In some embodiments, administration of the composition is given as a long-term treatment regimen whereby pharmacokinetic steady state conditions will be reached.

In general, by way of example only, dosage forms useful in the disclosed methods can include doses in the range of about 1 mg/kg to about 200 mg/kg, 10 mg/kg to 100 mg/kg, 20 mg/kg to 75 mg/kg, or 30 mg/kg to 60 mg/kg of body weight. In other embodiments, the dosage is about 200 mg/m2 to about 1000 mg/m2, more preferably about 200, 250, 300, 350, 400, 450, 500, 600, 700, 800, 900, or 1000 mg/m2. In some embodiments, the unit dosage is in a unit dosage form for intravenous injection. In some embodiments, the unit dosage is in a unit dosage form for intratumoral injection, intraperitoneal injection, or intravenous injection or infusion.

It will be appreciated that in some embodiments the effective amount of cell-penetrating binding protein, such as an antibody, and/or immune checkpoint modulator in a combination therapy may be different from that amount that would be effective for the cell-penetrating binding protein, such as an antibody, and immune checkpoint modulator to achieve the same result individually. For example, in some embodiments the effective amount of cell-penetrating binding protein, such as an antibody, and/or immune checkpoint modulator is a lower dosage of the cell-penetrating binding protein, such as an antibody, and/or immune checkpoint modulator in a combination therapy than the dosage of the cell-penetrating binding protein, such as an antibody, and/or immune checkpoint modulator that is effective when one agent is administered without the other. Alternatively, in some embodiments the effective amount of cell-penetrating binding protein, such as an antibody, and/or immune checkpoint modulator is a higher dosage of the cell-penetrating binding protein, such as an antibody, and/or immune checkpoint modulator in a combination therapy than the dosage of the cell-penetrating binding protein, such as an antibody, and/or immune checkpoint modulator that is effective when one agent is administered without the other. In other embodiments, the dosage of one agent is higher and the dosage of the other agent is lower than when one agent is administered without the other. In some cases, the agents are less effective, or not effective, when administered alone.

The frequency of administration can be, for example, one, two, three, four or more times daily, weekly, every two weeks, every three weeks, or monthly. In some embodiments, the inhibitor is administered to a subject once every 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days. In some embodiments, the frequency of administration is once weekly, or is once every two weeks, or is once every four weeks, or is twice every week. In some embodiments, a single administration is effective. In some embodiments two or more administrations are needed.

The compositions can be administered in a number of ways depending on whether local or systemic treatment is desired, and on the area to be treated. For example, the compositions may be administered enteral, including oral, parenteral (intramuscular, intraperitoneal, intravenous (IV), intrathecal, or subcutaneous injection), transdermal (either passively or using iontophoresis or electroporation), or transmucosal (nasal, vaginal, rectal, or sublingual) routes of administration or using bioerodible inserts and can be formulated in dosage forms appropriate for each route of administration.

The compositions may be administered directly into a tumor or tissue, e.g., stereotactically. In some embodiments, the compositions are administered into the brain or liver by injection or by a surgically implanted shunt.

In preferred embodiments, the composition is administered to the subject by injection or infusion. In a particular embodiment, the injection is a bolus injection. In another preferred embodiment, the pharmaceutical composition is administered to the subject by intravenous infusion. The infusion can be carried out over, seconds, minutes, or hours, for example, at least 1, 2, 3, 4, 5, 10, 30 or more seconds, at least 5, 10, 15, 30, 45, or 60 minutes, or about 1, 1.5, 2, 3, 4, 5 or more hours.

In some embodiments, the effect of the composition on a subject is compared to a control. For example, the effect of the composition on a particular symptom, pharmacologic, or physiologic indicator can be compared to an untreated subject, or the condition of the subject prior to treatment. In some embodiments, the symptom, pharmacologic, or physiologic indicator is measured in a subject prior to treatment, and again one or more times after treatment is initiated. In some embodiments, the control is a reference level, or average determined based on measuring the symptom, pharmacologic, or physiologic indicator in one or more subjects that do not have the disease or condition to be treated (e.g., healthy subjects). In some embodiments, the effect of the treatment is compared to a conventional treatment that is known the art, such as one of those discussed herein.

### III. Methods of Use

### A. Methods of Treatment

Methods of treating cancer and infections in a subject are provided. In certain embodiments, the methods include administering to a subject with cancer or an infection an effective amount of cell-penetrating binding protein, such as an antibody, in combination with one or more immune checkpoint modulators to reduce or inhibit one or more symptoms of the cancer or infection. In preferred embodiments, the cell-penetrating binding protein, such as an antibody, and immune checkpoint modulator can be used in combination to provide enhanced antitumor activity as compared to the use of either agent alone. The methods can include contacting one or more cancer cells or infected cells with an effective amount of a cell-penetrating binding protein, such as an antibody, in combination with one or more immune checkpoint modulators to decrease or inhibit the proliferation and/or viability of the cells compared to untreated control cells.

In some embodiments, methods of treating cancer defined herein encompass administering an above referenced cell-penetrating anti-DNA binding protein and an immune checkpoint modulator in combination. For example, the method of treating cancer can include administering an anti-DNA binding protein which includes a V_{H} including an amino acid sequence as shown in any one of SEQ ID NOs: 9, 11, or 45 to 52 and a V_{L} including an amino acid sequence as shown in any one of SEQ ID NOs: 3 to 5 or 53 to 58 and an immune checkpoint modulator in combination. In some embodiments, the method includes administering an anti-DNA binding protein which includes a V_{H} including an amino acid sequence as shown in SEQ ID NO:50 and a V_{L} including an amino acid sequence as shown in SEQ ID NO:56 and an immune checkpoint modulator in combination.

In other embodiments, methods of treating cancer defined herein encompass administering an anti-DNA binding protein which includes an amino acid sequence as shown in any one of SEQ ID NOs: 61-76 and an immune checkpoint modulator in combination. For example, the method can include administering an anti-DNA binding protein which includes an amino acid sequence as shown in SEQ ID NO:70 and an immune checkpoint modulator in combination.

Various examples of immune checkpoint modulators are disclosed herein. Referring to the above referenced embodiments, examples of immune checkpoint modulators include antibodies. For example, immune checkpoint modulators administered in combination with an above referenced anti-DNA binding protein to treat cancer can include Atezolizumab, Avelumab, Durvalumab, Ipilimumab, Nivolumab and Pembrolizumab. In an example, the immune checkpoint modulator is Pembrolizumab. In an example, the immune checkpoint modulator is a CAR-T cell. In another example, the immune checkpoint modulator is an oncolytic virus.

In some embodiments, the method of treating cancer can include administering an anti-DNA binding protein which includes a V_{H} including an amino acid sequence as shown in any one of SEQ ID NOs: 9, 11, or 45 to 52 and a V_{L} including an amino acid sequence as shown in any one of SEQ ID NOs: 3 to 5 or 53 to 58 and an immune checkpoint modulator which is an anti-PD1 an anti-PDL1, or an anti-CTLA4 antibody in combination. In some embodiments, the method includes administering an anti-DNA binding protein which includes a V_{H} including an amino acid sequence as shown in SEQ ID NO:50 and a V_{L} including an amino acid sequence as shown in SEQ ID NO:56 and an immune checkpoint modulator which is an anti-PD1 an anti-PDL1, or an anti-CTLA4 antibody in combination. In other embodiments, methods of treating cancer defined herein encompass administering an anti-DNA binding protein which includes an amino acid sequence as shown in any one of SEQ ID NOs: 61-76 and an immune checkpoint modulator which is an anti-PD1 an anti-PDL1, or an anti-CTLA4 antibody in combination. For example, the method can include administering an anti-DNA binding protein which includes an amino acid sequence as shown in SEQ ID NO:70 and an immune checkpoint modulator which is an anti-PD1 an anti-PDL1, or an anti-CTLA4 antibody in combination. In these embodiments the anti-PD1 antibody can be Pembrolizumab.

The cell-penetrating binding protein, such as an antibody, and/or immune checkpoint modulator can be administered locally or systemically to the subject, or coated or incorporated onto, or into a device.

The disclosed combination therapies and treatment regimens typically include treatment of a disease or symptom thereof, or a method for achieving a desired physiological change, including administering to an animal, such as a mammal, especially a human being, an effective amount of cell-penetrating binding protein, such as an antibody, and immune checkpoint modulator to treat a disease such as cancer or infection or symptom thereof, or to produce the physiological change, wherein the chemical agents or components are administered together, such as part of the same composition, or administered separately and independently at the same time or at different times (i.e., administration of the cell-penetrating binding protein, such as an antibody, and immune checkpoint modulator is separated by a finite period of time from each other). Therefore, the term “combination” or “combined” is used to refer to either concomitant, simultaneous, or sequential administration of the cell-penetrating binding protein, such as an antibody, and immune checkpoint modulator. The combinations can be administered either concomitantly (e.g., as an admixture), separately but simultaneously (e.g., via separate intravenous lines into the same subject; one agent is given orally while the other agent is given by infusion or injection, etc.,), or sequentially (e.g., one agent is given first followed by the second).

When used for treating cancer, the amount of cell-penetrating binding protein, such as an antibody, present in a pharmaceutical dosage unit, or otherwise administered to a subject can be an amount effective to induce or increase DNA damage in cells such as cancer or infected cells or reduce or otherwise impair DNA damage repair in cells such as cancer or infected cells alone or when administered in combination with an immune checkpoint modulator. Likewise, the amount of immune checkpoint modulator present in a pharmaceutical dosage unit, or otherwise administered to a subject can be an amount effective to induce or increase an immune response including by reducing suppression of immune response against the cancer cell or infected cells when administered alone in combination with a cell-penetrating binding protein, such as an antibody.

Therefore, in some embodiments the amount of the active agents is effective to reduce, slow or halt tumor progression or infection, to reduce tumor burden, or a combination thereof. In some embodiments, the amount of the active agents is effective to alter a measureable biochemical or physiological marker. For example, in some embodiments, the active agents increase the presences of cytoplasmic DNA (e.g., fragmented DNA), increase the appearance of DNA damage-repair foci (e.g., γH2AX foci), increase p21 protein level, increase p27 protein level, increase phosphorylation of STAT1, or any combination thereof.

In preferred embodiments, administration of a combination of a cell-penetrating binding protein, such as an antibody, and an immune checkpoint modulator such as those provided herein achieves a result greater than when the cell-penetrating binding protein, such as an antibody, and an immune checkpoint modulator are administered alone or in isolation. For example, in some embodiments, the result achieved by the combination is partially or completely additive of the results achieved by the individual components alone. In some embodiments, the result achieved by the combination is more than additive of the results achieved by the individual components alone. In some embodiments, the effective amount of one or both agents used in combination is lower than the effective amount of each agent when administered separately. In some embodiments, the amount of one or both agents when used in the combination therapy is sub-therapeutic when used alone.

The effect of the combination therapy, or individual agents thereof can depend on the disease or condition to be treated or progression thereof. For example, in some embodiments, the combination expands the subjects (e.g., the types of cancer or infection) that can be treated relative the each of the agents alone. Accordingly, in some embodiments, the effect of the combination on a cancer can compared to the effect of the individual agents alone on the cancer.

A treatment regimen of the combination therapy can include one or multiple administrations of a cell-penetrating binding protein, such as an antibody. A treatment regimen of the combination therapy can include one or multiple administrations of an immune checkpoint modulator. In certain embodiments, cell-penetrating binding protein, such as an antibody, can be administered simultaneously with an immune checkpoint modulator. Where a cell-penetrating binding protein, such as an antibody, and an immune checkpoint modulator are administered at the same time, the cell-penetrating binding protein, such as an antibody, and an immune checkpoint modulator can be, but need not be, in the same pharmaceutical composition.

In some embodiments cell-penetrating binding protein, such as an antibody, and an immune checkpoint modulator are administered sequentially, for example, in two or more different pharmaceutical compositions. In certain embodiments, the cell-penetrating binding protein, such as an antibody, is administered prior to the first administration of the immune checkpoint modulator. In other embodiments, the immune checkpoint modulator is administered prior to the first administration of the cell-penetrating binding protein, such as an antibody. For example, the cell-penetrating binding protein, such as an antibody, and the immune checkpoint modulator can be administered to a subject on the same day.

Alternatively, the cell-penetrating binding protein, such as an antibody, and the immune checkpoint modulator can be administered to the subject on different days.

The cell-penetrating binding protein, such as an antibody, can be administered at least 1, 2, 3, 5, 10, 15, 20, 24 or 30 hours or days prior to or after administering of the immune checkpoint modulator. Alternatively, the immune checkpoint modulator can be administered at least 1, 2, 3, 5, 10, 15, 20, 24 or 30 hours or days prior to or after administering of the cell-penetrating binding protein, such as an antibody. In certain embodiments, additive or more than additive effects of the administration of cell-penetrating binding protein, such as an antibody, in combination with immune checkpoint modulator is evident after one day, two days, three days, four days, five days, six days, one week, or more than one week following administration.

Dosage regimens or cycles of the agents can be completely or partially overlapping, or can be sequential. For example, in some embodiments, all such administration(s) of the cell-penetrating binding protein, such as an antibody, occur before or after administration of the immune checkpoint modulator. Alternatively, administration of one or more doses of the cell-penetrating binding protein, such as an antibody, can be temporally staggered with the administration of immune checkpoint inhibitor to form a uniform or non-uniform course of treatment whereby one or more doses of cell-penetrating binding protein, such as an antibody, are administered, followed by one or more doses of immune checkpoint modulator, followed by one or more doses of cell-penetrating binding protein, such as an antibody,; or one or more doses of immune checkpoint modulator are administered, followed by one or more doses of cell-penetrating binding protein, such as an antibody,, followed by one or more doses of immune checkpoint modulator; etc., all according to whatever schedule is selected or desired by the researcher or clinician administering the therapy.

An effective amount of each of the agents can be administered as a single unit dosage (e.g., as dosage unit), or sub-therapeutic doses that are administered over a finite time interval. Such unit doses may be administered on a daily basis for a finite time period, such as up to 3 days, or up to 5 days, or up to 7 days, or up to 10 days, or up to 15 days or up to 20 days or up to 25 days, are all specifically contemplated.

1. Cancer

The combination therapies disclosed herein can be used to treat, reduce, and/or prevent cancer in a subject. Therefore, the combination can be administered in an effective amount to treat, reduce, and/or prevent cancer in a subject. The effective amount or therapeutically effective amount of the combination to treat cancer or a tumor thereof is typically a dosage sufficient to reduce or prevent a least one symptom of the cancer, or to otherwise provide a desired pharmacologic and/or physiologic effect. The symptom may be physical, such as tumor burden, or biological such as reducing proliferation or increasing death of cancer cells. In some embodiments, the amount is effective to kill tumor cells or reduce or inhibit proliferation or metastasis of the tumor cells. In some embodiments, the amount is effective to reduce tumor burden. In some embodiments, the amount is effective to reduce or prevent at least one comorbidity of the cancer.

In a mature animal, a balance usually is maintained between cell renewal and cell death in most organs and tissues. The various types of mature cells in the body have a given life span; as these cells die, new cells are generated by the proliferation and differentiation of various types of stem cells. Under normal circumstances, the production of new cells is so regulated that the numbers of any particular type of cell remain constant. Occasionally, though, cells arise that are no longer responsive to normal growth-control mechanisms. These cells give rise to clones of cells that can expand to a considerable size, producing a tumor or neoplasm. A tumor that is not capable of indefinite growth and does not invade the healthy surrounding tissue extensively is benign. A tumor that continues to grow and becomes progressively invasive is malignant. The term cancer typically refers to a malignant tumor. In addition to uncontrolled growth, malignant tumors exhibit metastasis. In this process, small clusters of cancerous cells dislodge from a tumor, invade the blood or lymphatic vessels, and are carried to other tissues, where they continue to proliferate. In this way a primary tumor at one site can give rise to a secondary tumor at another site.

The compositions and methods described herein are useful for treating subjects having benign or malignant tumors by delaying or inhibiting the growth of a tumor in a subject, reducing the growth or size of the tumor, inhibiting or reducing metastasis of the tumor, and/or inhibiting or reducing symptoms associated with tumor development or growth.

Malignant tumors which may be treated can be classified according to the embryonic origin of the tissue from which the tumor is derived. Carcinomas are tumors arising from endodermal or ectodermal tissues such as skin or the epithelial lining of internal organs and glands. The disclosed compositions are particularly effective in treating carcinomas. Sarcomas, which arise less frequently, are derived from mesodermal connective tissues such as bone, fat, and cartilage. The leukemias and lymphomas are malignant tumors of hematopoietic cells of the bone marrow. Leukemias proliferate as single cells, whereas lymphomas tend to grow as tumor masses. Malignant tumors may show up at numerous organs or tissues of the body to establish a cancer.

The disclosed antigen binding molecules can be used to treat cells undergoing unregulated growth, invasion, or metastasis.

Tumor cell hypoxia is now recognized as a problem in cancer therapy because it makes cancer cells resistant to treatment with radiation and some chemotherapeutics. Hypoxia is also known to cause impaired DNA repair in cancer cells. Accordingly, in some embodiments, the disclosed active agents are used as targeted agents for hypoxic tumor cells.

Cancer cells that have impaired DNA repair are particularly good targets for the disclosed compositions. In some embodiments, the compositions are lethal to cells with impaired DNA repair. In preferred embodiments, the cells are defective in the expression of a gene or in the function of a protein involved in DNA repair, DNA synthesis, or homologous recombination. Exemplary genes and associated products include XRCC1, ADPRT (PARP-1), ADPRTL2, (PARP-2), POLYMERASE BETA, CTPS, MLH1, MSH2, FANCD2, PMS2, p53, p21, PTEN, RPA, RPAI, RPA2, RPA3, XPD, ERCC1, XPF, MMS19, RAD51, RAD51B, RAD51C, RAD51D, DMC1, XRCCR, XRCC3, BRCA1, BRCA2, PALB2, RAD52, RAD54, RAD50, MRE11, NB51, WRN, BLM, KU70, KU80, ATM, ATR CHK1, CHK2, FANC family of genes, FANCA, FANCB, FANCC, FANCDI, FANCD2, FANCE, FANCF, FANCG, FANCL, FANCM, RAD1, and RAD9.

In some embodiments, the defective gene is a tumor suppressor gene. In some embodiments, the gene is associated with maintenance of chromosomal integrity and/or protection from genotoxic stress. In a most preferred embodiment, the cells are deficient in single and/or double strand break repair.

In preferred embodiments, the cells have one or more mutations in BRCA1, BRCA2, and/or PTEN. Gene mutations, such as BRCA1, BRCA2, PTEN mutations, can be identified using standard PCR, hybridization, or sequencing techniques.

In particular embodiments, the cancer cell is deficient in DNA damage repair due to hypoxia.

Therefore, in some embodiments, the antigen binding molecules can be used to treat cancers arising from DNA repair deficient familial syndromes, such as breast, ovarian, and pancreatic cancers. In these embodiments, the anti-DNA antibodies can be effective without radiotherapy or chemotherapy. For example, the antigen binding molecules can be used to treat cancers that are linked to mutations in BRCA1, BRCA2, PALB2, or RAD51B, RAD51C, RAD51D, or related genes. The antigen binding molecules can also be used to treat colon cancers, endometrial tumors, or brain tumors linked to mutations in genes associated with DNA mismatch repair, such as MSH2, MLH1, PMS2, and related genes. The antigen binding molecules can also be used to treat cancers with silenced DNA repair genes, such as BRCA1, MLH1, OR RAD51B, RAD51C, or RAD51D. The antigen binding molecules can also be used to treat cancers associated with chromosomal maintenance or genotoxic stress, for example, cancers in which PTEN is mutated or silenced. PTEN is frequently inactivated in many cancers including breast, prostate, glioma, melanoma, and lung cancers. In these preferred embodiments, the ability of the antigen binding molecules to impair DNA repair combined with the inherent repair deficiencies or other susceptibilities of these cancers can be sufficient to induce cell death.

A representative but non-limiting list of cancers that the compositions can be used to treat include cancers of the blood and lymphatic system (including leukemias, Hodgkin's lymphomas, non-Hodgkin's lymphomas, solitary plasmacytoma, multiple myeloma), cancers of the genitourinary system (including prostate cancer, bladder cancer, renal cancer, urethral cancer, penile cancer, testicular cancer,), cancers of the nervous system (including mengiomas, gliomas, glioblastomas, ependymomas) cancers of the head and neck (including squamous cell carcinomas of the oral cavity, nasal cavity, nasopharyngeal cavity, oropharyngeal cavity, larynx, and paranasal sinuses), lung cancers (including small cell and non-small cell lung cancer), gynecologic cancers (including cervical cancer, endometrial cancer, vaginal cancer, vulvar cancer ovarian and fallopian tube cancer), gastrointestinal cancers (including gastric, small bowel, colorectal, liver, hepatobiliary, and pancreatic cancers), skin cancers (including melanoma, squamous cell carcinomas, and basal cell carcinomas), breast cancer (including ductal and lobular cancer and triple negative breast cancers), and pediatric cancers (including neuroblastoma, Ewing's sarcoma, Wilms tumor, medulloblastoma). Accordingly, in some embodiments, the present disclosure relates to a method of treating breast, ovarian, colon, prostate, lung, brain, skin, liver, stomach, pancreatic or blood based cancer. In some embodiments, the present disclosure relates to treating glioblastoma. In this example, glioblastoma may be treated by administering a binding protein disclosed herein such as a di-scFv having SEQ ID NO:70 or an antibody having the heavy and light chain variable regions defined in SEQ ID NO:70 in combination with an immune checkpoint modulator.

In some embodiments, the cancer is a neoplasm or tumor that demonstrates some resistance to radiotherapy or chemotherapy. In particular embodiments, the cancer cell is resistant to radiation or chemotherapy due to hypoxia.

Cancers that are resistant to radiotherapy using standard methods include, but are not limited to, sarcomas, renal cell cancer, melanoma, lymphomas, leukemias, carcinomas, blastomas, and germ cell tumors.

### 2. Virally Transformed Cells

In some embodiments, the combination can be used to treat virally transformed cells, such as cells infected with an oncovirus. The effective amount or therapeutically effective amount to treat virally transfected cells is typically a dosage sufficient to kill the cells and/or sensitive them to another cytotoxic agent, or to otherwise provide a desired pharmacologic and/or physiologic effect. For example, viral transformation can impose phenotypic changes on cell, such as high saturation density, anchorage-independent growth, loss of contact inhibition, loss of orientated growth, immortalization, and disruption of the cell's cytoskeleton. The persistence of at least part of the viral genome within the cell is required for cell transformation. This may be accompanied by the continual expression from a number of viral genes, such as oncogenes. These genes may interfere with a cell's signaling pathway causing the observed phenotypic changes of the cell. In some cases, the viral genome is inserted near a proto-oncogene in the host genome. The end result is a transformed cell showing increased cell division, which is favorable to the virus. In some embodiments, viral transformation, viral infection, and/or metabolism is dependent upon DNA repair mechanisms. In these embodiments, inhibition of DNA repair using the disclosed antigen binding molecules also inhibits viral transformation, viral infection and/or metabolism in the cell.

In some embodiments, viral transformation, viral infection, and/or metabolism is dependent upon metabolism of the virally encoded RNA or DNA as a part of the virus life cycle, producing intermediates subject to binding and/or inhibition by the disclosed antibody fragments or fusion proteins. In these embodiments, treatment with the disclosed antigen binding molecules also inhibits viral transformation, viral infection and/or metabolism in the cell.

Lentiviruses (such as HIV) have been previously found to be dependent on host BER activity for infection and integration (Yoder et al., PLOS One, 2011 Mar. 6 (3) e17862). In addition, the ataxia-telangiectesia-mutated (ATM) DNA-damage response appears to be critical to HIV replication (Lau et al., Nat Cell Biol, 2005 7 (5): 493-500). In some embodiments, retroviral (including lentiviruses, HIV) infection and integration is dependent on host DNA repair mechanisms. In these embodiments treatment with the disclosed compositions can also suppresses viral infection/integration and suppresses re-infection in the viral life cycle.

In some embodiments, lentiviral (HIV) replication is dependent on DNA repair. In these embodiments treatment with the compositions also suppresses viral replication and suppresses re-infection in the viral life cycle. Therefore, the disclosed compositions can be used to treat cells infected with a virus, such as an oncovirus. In some embodiments, the composition inhibits viral transformation, replication, metabolism, or a combination thereof. Exemplary viruses that can be affected by disclosed compositions include Human papillomaviruses (HPV), Hepatitis B (HBV), Hepatitis C (HCV), Human T-lymphotropic virus (HTLV), Kaposi's sarcoma-associated herpesvirus (HHV-8), Merkel cell polyomavirus, Epstein-Barr virus (EBV), Human immunodeficiency virus (HIV), and Human cytomegalovirus (CMV). The antigen binding molecules may also be used to treat a latent virus. In some embodiments, the failure of infected cells to mount a DNA damage response to viruses, such as HSV-1, contribute to the establishment of latency. These virally infected cells therefore have impaired DNA repair and are susceptible to treatment with the disclosed compositions. Exemplary latent viruses include CMV, EBV, Herpes simplex virus (type 1 and 2), and Varicella zoster virus.

The disclosed compositions may also be used to treat active viral infections due to viruses that give rise to cancer, immunodeficiency, hepatitis, encephalitis, pneumonitis, respiratory illness, or other disease condition, by virtue of the cell-penetrating antibody's ability to bind to DNA and to interfere with DNA repair or RNA metabolisms that is part of the virus life cycle.

Representative viruses whose life cycle or symptoms of the resulting infection, that may be affected by administration of the antibodies include Human papillomaviruses (HPV), Hepatitis B (HBV), Hepatitis C (HCV), Human T-lymphotropic virus (HTLV), Kaposi's sarcoma-associated herpesvirus (HHV-8), Merkel cell polyomavirus, Epstein-Barr virus (EBV), Human immunodeficiency virus (HIV), and Human cytomegalovirus (CMV).

TABLE 1
Additional viruses that may be affected by administration of the
compositions include parvovirus, poxvirus, herpes virus, and other DNA viruses:

| | | Virion | | | |
|---|---|---|---|---|---|
| Virus | Examples | naked/ | Capsid | Nucleic | |
| Family | (common names) | enveloped | Symmetry | acid type | Group |
| 1.Adenoviridae | Adenovirus, | Naked | Icosahedral | ds | I |
| | Infectious canine | | | | |
| | hepatitis virus | | | | |
| 2.Papillomaviridae | Papillomavirus | Naked | Icosahedral | ds | I |
| | | | | circular | |
| 3.Parvoviridae | Parvovirus B19, | Naked | Icosahedral | ss | II |
| | Canine parvovirus | | | | |
| 4.Herpesviridae | Herpes simplex | Enveloped | Icosahedral | ds | I |
| | virus, varicella- | | | | |
| | zoster virus, | | | | |
| | cytomegalovirus, | | | | |
| | Epstein-Barr virus | | | | |
| 5.Poxviridae | Smallpox virus, | Complex | Complex | ds | I |
| | cow pox virus, | coats | | | |
| | sheep pox virus, | | | | |
| | monkey orf virus, | | | | |
| | pox virus, vaccinia virus | | | | |
| 6.Hepadnaviridae | Hepatitis B virus | Enveloped | Icosahedral | circular, | VII |
| | | | | partially | |
| | | | | ds | |
| 7.Polyomaviridae | Polyoma virus; JC | Naked | Icosahedral | ds | I |
| | virus (progressive | | | circular | |
| | multifocal | | | | |
| | leukoencephalopathy) | | | | |
| 8.Anelloviridae | Torque teno virus | | | | |

TABLE 2
RNA viruses that may be affected by administration of the compositions include:

| | | Capsid | | Nucleic | |
|---|---|---|---|---|---|
| Virus | Examples | naked/ | Capsid | acid | |
| Family | (common names) | enveloped | Symmetry | type | Group |
| 1.Reoviridae | Reovirus, Rotavirus | Naked | Icosahedral | ds | III |
| 2.Picornaviridae | Enterovirus, Rhinovirus, | Naked | Icosahedral | ss | IV |
| | Hepatovirus, | | | | |
| | Cardiovirus, | | | | |
| | Aphthovirus, Poliovirus, | | | | |
| | Parechovirus, Arbovirus, | | | | |
| | Kobuvirus, Teschovirus, | | | | |
| | Coxsackie | | | | |
| 3.Caliciviridae | Norwalk virus | Naked | Icosahedral | ss | IV |
| 4.Togaviridae | Rubella virus | Enveloped | Icosahedral | ss | IV |
| 5.Arenaviridae | Lymphocytic | Enveloped | Complex | ss(−) | V |
| | choriomeningitis virus | | | | |
| 6.Flaviviridae | Dengue virus, Hepatitis | Enveloped | Icosahedral | ss | IV |
| | C virus, Yellow fever virus | | | | |
| 7.0rthomyxoviridae | Influenzavirus A, | Enveloped | Helical | ss(−) | V |
| | Influenzavirus B, | | | | |
| | Influenzavirus C, | | | | |
| | Isavirus, Thogotovirus | | | | |
| 8.Paramyxoviridae | Measles virus, Mumps | Enveloped | Helical | s(−) | V |
| | virus, Respiratory | | | | |
| | syncytial virus, | | | | |
| | Rinderpest virus, Canine | | | | |
| | distemper virus | | | | |
| 9.Bunyaviridae | California encephalitis | Enveloped | Helical | ss(−) | V |
| | virus, Hantavirus | | | | |
| 10.Rhabdoviridae | Rabies virus | Enveloped | Helical | ss(−) | V |
| 11.Filoviridae | Ebola virus, Marburg | Enveloped | Helical | ss(−) | V |
| | virus | | | | |
| 12.Coronaviridae | Corona virus | Enveloped | Helical | ss | IV |
| 13.Astroviridae | Astrovirus | Naked | Icosahedral | ss | IV |
| 14.Bornaviridae | Borna disease virus | Enveloped | Helical | ss(−) | V |
| 15.Arteriviridae | Arterivirus, Equine | Enveloped | Icosahedral | ss | IV |
| | Arteritis Virus | | | | |
| 16.Hepeviridae | Hepatitis E virus | Naked | Icosahedral | ss | IV |

### Retroviruses May Also be Affected:

Genus Alpharetrovirus; type species: Avian leukosis virus; others include Rous sarcoma virus
Genus Betaretrovirus; type species: Mouse mammary tumor virus
Genus Gammaretrovirus; type species: Murine leukemia virus; others include Feline leukemia virus
Genus Deltaretrovirus; type species: Bovine leukemia virus; others include the cancer-causing Human T-lymphotropic virus
Genus Epsilonretrovirus; type species: Walleye dermal sarcoma virus
Genus Lentivirus; type species: Human immunodeficiency virus 1 and human immunodeficiency virus 2; others include Simian, Feline immunodeficiency viruses Genus Spumavirus; type species: Simian foamy virus Family Hepadnaviridae—e.g. Hepatitis B virus

Other viral diseases that may be affected by administration of the compositions include Colorado Tick Fever (caused by Coltivirus, RNA virus), West Nile Fever (encephalitis, caused by a flavivirus that primarily occurs in the Middle East and Africa), Yellow Fever, Rabies (caused by a number of different strains of neurotropic viruses of the family Rhabdoviridae), viral hepatitis, gastroenteritis (viral)-acute viral gastroenteritis caused by Norwalk and Norwalk-like viruses, rotaviruses, caliciviruses, and astroviruses, poliomyelitis, influenza (flu), caused by orthomyxoviruses that can undergo frequent antigenic variation, measles (rubella), paramyxoviridae, mumps, respiratory syndromes including viral pneumonia and acute respiratory syndromes including croup caused by a variety of viruses collectively referred to as acute respiratory viruses, and respiratory illness caused by the respiratory syncytial virus (RSV, the most dangerous cause of respiratory infection in young children).

In some embodiments, the disclosed compositions are used to treat or prevent a viral infection or the spread or worsening of a viral infection. For example, in some embodiments, the compositions are used to treat or prevent a viral infection or the spread or worsening of a viral infection in a subject that has been exposed to or is at risk of being exposed to a virus, such as those discussed herein.

### B. Combination Therapies

The combination of a cell-penetrating binding protein and an immune checkpoint modulator may also potentiate other active agents and therapies, resulting further improved, additive or more than additive treatment results. In some embodiments, the disclosed combination therapies are used in further combination with radiotherapy, chemotherapy, or a combination thereof, to treat any cancer, including carcinomas, gliomas, sarcomas, or lymphomas. In these embodiments, the disclosed compositions can sensitize the cells to the DNA-damaging effects of radiotherapy or chemotherapy.

The disclosed compositions can increase a cancer's radiosensitivity or chemosensitivity. Effective doses of chemotherapy and/or radiation therapy may be toxic for certain cancers. In some embodiments, the compositions decrease the required effective dose of an anti-neoplastic drug or radiation levels needed to treat a cancer, thereby reducing toxicity of the effective dose. For example, the most commonly used dosage of doxorubicin is 40 to 60 mg/m² IV every 21 to 28 days, or 60 to 75 mg/m² IV once every 21 days. If the patient has a bilirubin level between 1.2 and 3 mg/dL, the dose should be reduced by 50%. If the patient has a bilirubin level between 3.1 and 5.0 mg/dL, the dose should be reduced by 75%. Serious irreversible myocardial toxicity leading to congestive heart failure often unresponsive to cardiac support therapy may be encountered as the total dosage of doxorubicin approaches 450 mg/m². When used in combination with the disclosed compositions, doxorubicin dosage may be reduced to decrease myocardial toxicity without a loss in efficacy.

In other embodiments, the disclosed compositions may be used with normal doses of drug or radiation to increase efficacy. For example, a cell-penetrating binding protein, such as an antibody, and/or immune checkpoint modulator may be used to potentiate a drug or radiation therapy for a cancer that is drug or radiation resistant. Cancers that are resistant to radiotherapy using standard methods include sarcomas, melanomas, carcinomas, and hypoxic tumors.

### 1. Radiotherapy

The disclosed combination therapies can be used in further combination with radiation therapy. Radiation therapy (a.k.a. radiotherapy) is the medical use of ionizing radiation as part of cancer treatment to control malignant cells. Radiotherapy also has several applications in non-malignant conditions, such as the treatment of trigeminal neuralgia, severe thyroid eye disease, pterygium, pigmented villonodular synovitis, prevention of keloid scar growth, and prevention of heterotopic ossification. In some embodiments, the disclosed compositions are used to increase radiosensitivity for a non-malignant condition.

Radiation therapy works by damaging the DNA of dividing cells, e.g., cancer cells. This DNA damage is caused by one of two types of energy, photon or charged particle. This damage is either direct or indirect. Indirect ionization happens as a result of the ionization of water, forming free radicals, notably hydroxyl radicals, which then damage the DNA. For example, most of the radiation effect caused by photon therapy is through free radicals. One of the major limitations of photon radiotherapy is that the cells of solid tumors become deficient in oxygen, and tumor cells in a hypoxic environment may be as much as 2 to 3 times more resistant to radiation damage than those in a normal oxygen environment.

Direct damage to cancer cell DNA occurs through high-LET (linear energy transfer) charged particles such as proton, boron, carbon or neon ions. This damage is independent of tumor oxygen supply because these particles act mostly via direct energy transfer usually causing double-stranded DNA breaks. Due to their relatively large mass, protons and other charged particles have little lateral side scatter in the tissue; the beam does not broaden much, stays focused on the tumor shape and delivers small dose side-effects to surrounding tissue. The amount of radiation used in photon radiation therapy is measured in Gray (Gy), and varies depending on the type and stage of cancer being treated. For curative cases, the typical dose for a solid epithelial tumor ranges from 60 to 70 Gy, while lymphomas are treated with lower doses.

In some cases, solid tumors are treated with stereotactic body radiation therapy (SBRT) in which several large single doses are given with high precision, for example 20 Gy x3 doses, 18 Gy x 3 doses, and 10 Gy x 5 doses. This treatment method is sometimes referred to as hypofractionation. Hypofractionated SBRT treatments are can be combined with immune checkpoint therapy. It is believed that this combination enhances tumor immunogenicity and enhances the immune response to the tumor (Popp, et al., Radiotherapy and Oncology, 120 (2016) 185-194).

Post-operative (adjuvant) doses are typically around 45-60 Gy in 1.8-2 Gy fractions (for breast, head, and neck cancers). Many other factors are considered by radiation oncologists when selecting a dose, including whether the patient is receiving chemotherapy, patient co-morbidities, whether radiation therapy is being administered before or after surgery, and the degree of success of surgery.

The response of a cancer to radiation is described by its radiosensitivity. Highly radiosensitive cancer cells are rapidly killed by modest doses of radiation. These include leukemias, most lymphomas and germ cell tumors. The majority of epithelial cancers are only moderately radiosensitive, and require a significantly higher dose of radiation (60-70 Gy) to achieve a radical cure. Some types of cancer are notably radioresistant, that is, much higher doses are required to produce a radical cure than may be safe in clinical practice. Renal cell cancer and melanoma are generally considered to be radioresistant.

The response of a tumor to radiotherapy is also related to its size. For complex reasons, very large tumors respond less well to radiation than smaller tumors or microscopic disease. Various strategies are used to overcome this effect. The most common technique is surgical resection prior to radiotherapy. This is most commonly seen in the treatment of breast cancer with wide local excision or mastectomy followed by adjuvant radiotherapy. Another method is to shrink the tumor with neoadjuvant chemotherapy prior to radical radiotherapy. A third technique is to enhance the radiosensitivity of the cancer by giving certain drugs during a course of radiotherapy. The disclosed antigen binding molecules can serve this third function. In these embodiments, the antigen binding molecule can increase the cell's sensitivity to the radiotherapy, for example, by at least 10%, 15%, 20%, 25%, 30%, 35%, 40%, 45%, 50%. Moreover, the antigen binding molecule can be combined with one or more additional radiosensitizers. Examples of known radiosensitizers include cisplatin, gemcitabine, 5-fluorouracil, pentoxifylline, vinorelbine, PARP inhibitors, histone deacetylase inhibitors, and proteasome inhibitors.

In other embodiments, the dose of radiation can be reduced byl0%, 15%, 20%, 25%, 30%, 35%, 40%, 45%, 50%, or more when administered in combination with the disclosed antigen binding molecules.

### 2. Chemotherapeutics

Numerous chemotherapeutics, especially antineoplastic drugs, are available for further combination with the disclosed combination therapies. The majority of chemotherapeutic drugs can be divided into alkylating agents, antimetabolites, anthracyclines, plant alkaloids, topoisomerase inhibitors, monoclonal antibodies, and other antitumor agents.

In preferred embodiments, the antineoplastic drug damages DNA or interferes with DNA repair since these activities may enhance the disclosed combination therapies. In these embodiments, the combination may increase the cell's sensitivity to the chemotherapy, for example, by at least 10%, 15%, 20%, 25%, 30%, 35%, 40%, 45%, 50%. Non-limiting examples of antineoplastic drugs that damage DNA or impair DNA repair include carboplatin, carmustine, chlorambucil, cisplatin, cyclophosphamide, dacarbazine, daunorubicin, doxorubicin, epirubicin, idarubicin, ifosfamide, lomustine, mechlorethamine, mitoxantrone, oxaliplatin, procarbazine, temozolomide, and valrubicin. In some embodiments, the antineoplastic drug is temozolomide, which is a DNA damaging alkylating agent commonly used against glioblastomas. In some embodiments, the antineoplastic drug is a PARP inhibitor, which inhibits a step in base excision repair of DNA damage. In some embodiments, the antineoplastic drug is a histone deacetylase inhibitor, which suppresses DNA repair at the transcriptional level and disrupt chromatin structure. In some embodiments, the antineoplastic drug is a proteasome inhibitor, which suppresses DNA repair by disruption of ubiquitin metabolism in the cell. Ubiquitin is a signaling molecule that regulates DNA repair. In some embodiments, the antineoplastic drug is a kinase inhibitor, which suppresses DNA repair by altering DNA damage response signaling pathways.

In other embodiments, the antineoplastic drug complements the cell-penetrating binding protein, such as an antibody, and/or the immune checkpoint modulator by targeting a different activity in the cancer cell. In these embodiments, the antineoplastic drug does not impair DNA repair or damage DNA.

Examples of antineoplastic drugs that can be combined with the disclosed antigen binding molecules include, but are not limited to, alkylating agents (such as temozolomide, cisplatin, carboplatin, oxaliplatin, mechlorethamine, cyclophosphamide, chlorambucil, dacarbazine, lomustine, carmustine, procarbazine, chlorambucil and ifosfamide), antimetabolites (such as fluorouracil, gemcitabine, methotrexate, cytosine arabinoside, fludarabine, and floxuridine), some antimitotics, and vinca alkaloids such as vincristine, vinblastine, vinorelbine, and vindesine), anthracyclines (including doxorubicin, daunorubicin, valrubicin, idarubicin, and epirubicin, as well as actinomycins such as actinomycin D), cytotoxic antibiotics (including mitomycin, plicamycin, and bleomycin), and topoisomerase inhibitors (including camptothecins such as irinotecan and topotecan and derivatives of epipodophyllotoxins such as amsacrine, etoposide, etoposide phosphate, and teniposide).

In other embodiments, the dose of chemotherapy can be reduced by 10%, 15%, 20%, 25%, 30%, 35%, 40%, 45%, 50%, or more when administered in combination with the disclosed compositions.

### IV. Methods of Making and Isolating Binding Proteins

### A. Binding Protein Production

### 1. Recombinant Expression

In one example, a binding protein as described herein is a peptide or polypeptide (e.g., is an antibody or antigen binding fragment thereof). In one example, the binding protein is recombinant.

In the case of a recombinant peptide or polypeptide, nucleic acid encoding same can be cloned into expression vectors, which are then transfected into host cells, such as E. coli cells, yeast cells, insect cells, or mammalian cells, such as simian COS cells, Chinese Hamster Ovary (CHO) cells, human embryonic kidney (HEK) cells, or myeloma cells that do not otherwise produce immunoglobulin or antibody protein.

Suitable molecular cloning techniques are known in the art and described, for example in Ausubel et al., (editors), Current Protocols in Molecular Biology, Greene Pub. Associates and Wiley-Interscience (1988, including all updates until present) or Sambrook et al., Molecular Cloning: A Laboratory Manual, Cold Spring Harbor Laboratory Press (1989). A wide variety of cloning and in vitro amplification methods are suitable for the construction of recombinant nucleic acids. Methods of producing recombinant antibodies are also known in the art. See U.S. Pat. No. 4,816,567 or U.S. Pat. No. 5,530,101.

Following isolation, the nucleic acid is inserted operably linked to a promoter in an expression construct or expression vector for further cloning (amplification of the DNA) or for expression in a cell-free system or in cells. Thus, another example of the disclosure provides an expression construct that includes an isolated nucleic acid of the disclosure and one or more additional nucleotide sequences. Suitably, the expression construct is in the form of, or includes genetic components of, a plasmid, bacteriophage, a cosmid, a yeast or bacterial artificial chromosome as are understood in the art. Expression constructs may be suitable for maintenance and propagation of the isolated nucleic acid in bacteria or other host cells, for manipulation by recombinant DNA technology and/or for expression of the nucleic acid or a binding protein of the disclosure.

Many vectors for expression in cells are available. The vector components generally include, but are not limited to, one or more of the following: a signal sequence, a sequence encoding the binding protein (e.g., derived from the information provided herein), an enhancer element, a promoter, and a transcription termination sequence. Exemplary signal sequences include prokaryotic secretion signals (e.g., pelB, alkaline phosphatase, penicillinase, Ipp, or heat-stable enterotoxin II), yeast secretion signals (e.g., invertase leader, a factor leader, or acid phosphatase leader) or mammalian secretion signals (e.g., herpes simplex gD signal).

Exemplary promoters active in mammalian cells include cytomegalovirus immediate early promoter (CMV-IE), human elongation factor 1-α promoter (EF1), small nuclear RNA promoters (U1a and U1b), α-myosin heavy chain promoter, Simian virus 40 promoter (SV40), Rous sarcoma virus promoter (RSV), Adenovirus major late promoter, β-actin promoter; hybrid regulatory element including a CMV enhancer/β-actin promoter or an immunoglobulin or antibody promoter or active fragment thereof. Examples of useful mammalian host cell lines are monkey kidney CV1 line transformed by SV40 (COS-7, ATCC CRL 1651); human embryonic kidney line (293 or 293 cells subcloned for growth in suspension culture; baby hamster kidney cells (BHK, ATCC CCL 10); or Chinese hamster ovary cells (CHO).

Typical promoters suitable for expression in yeast cells such as for example a yeast cell selected from the group including Pichia pastoris, Saccharomyces cerevisiae and S. pombe, include, but are not limited to, the ADHI promoter, the GALI promoter, the GAL4 promoter, the CUP1 promoter, the PHO5 promoter, the nmt promoter, the RPR1 promoter, or the TEF1 promoter.

Means for introducing the isolated nucleic acid or expression construct including same into a cell for expression are known to those skilled in the art. The technique used for a given cell depends on the known successful techniques. Means for introducing recombinant DNA into cells include microinjection, transfection mediated by DEAE-dextran, transfection mediated by liposomes such as by using lipofectamine (Gibco, MD, USA) and/or cellfectin (Gibco, MD, USA), PEG-mediated DNA uptake, electroporation and microparticle bombardment such as by using DNA-coated tungsten or gold particles (Agracetus Inc., WI, USA) amongst others.

The host cells used to produce the binding protein (e.g., antibody or antigen binding fragment) may be cultured in a variety of media, depending on the cell type used. Commercially available media such as Ham's F10 (Sigma), Minimal Essential Medium ((MEM), (Sigma), RPM1-1640 (Sigma), and Dulbecco's Modified Eagle's Medium ((DMEM), Sigma) are suitable for culturing mammalian cells. Media for culturing other cell types discussed herein are known in the art.

The skilled artisan will understand from the foregoing description that the present disclosure also provides an isolated nucleic acid encoding a binding protein (e.g., a peptide or polypeptide binding protein or an antibody or antigen binding fragment thereof) of the present disclosure.

The present disclosure also provides an expression construct including an isolated nucleic acid of the disclosure operably linked to a promoter. In one example, the expression construct is an expression vector.

In one example, the expression construct of the disclosure includes a nucleic acid encoding a polypeptide (e.g., including a V_{H}) operably linked to a promoter and a nucleic acid encoding another polypeptide (e.g., including a V_{L}) operably linked to a promoter.

The disclosure also provides a host cell including an expression construct according to the present disclosure.

The present disclosure also provides an isolated cell expressing a binding protein of the disclosure or a recombinant cell genetically-modified to express the binding protein.

### 2. Isolation of Proteins

Methods for purifying binding proteins according to the present disclosure are known in the art and/or described herein.

Where a peptide or polypeptide is secreted into the medium, supernatants from such expression systems can be first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The binding protein prepared from cells can be purified using, for example, ion exchange, hydroxyapatite chromatography, hydrophobic interaction chromatography, gel electrophoresis, dialysis, affinity chromatography (e.g., protein A affinity chromatography or protein G chromatography), or any combination of the foregoing. These methods are known in the art and described, for example in WO99/57134 or Ed Harlow and David Lane (editors) Antibodies: A Laboratory Manual, Cold Spring Harbor Laboratory, (1988).

The present invention will be further understood by reference to the following non-limiting examples.

### EXAMPLES

### Example 1: 3E10 Increases STAT1 Phosphorylation in Cancer Cells

### Materials and Methods

Cell culture and treatment

Cancer cells were seeded at a density of 50,000 cells/well of a 6 well plate in DMEM media, containing 10% FBS and were then incubated at 37° C. in 5% CO2. 24 hours after seeding, cells were treated with 3E10 (either 3E10 WT, 3E10 D31N, or a truncated version of 3E10) by simple addition to the culture medium. The final concentration of antibody in each case was 1 μM.

Three days after treatment, whole cell lysates were prepared by harvesting the cells via trypsinization and pelleted via centrifugation. Cell pellets were lysed in AZ lysis buffer (50 mM Tris pH 8, 250 mM NaCl, 1% NP-40, 0.1% SDS, 5 mM EDTA, 10 mM Na4P207, 10 mM NaF, 1× complete EDTA-free Protease Inhibitor Cocktail (Roche), 1× PhosSTOP (Roche)). The protein concentration of each sample was determined using the DC™ (detergent compatible) protein assay (Bio-Rad Laboratories, Inc.). Protein concentrations were normalized and samples were prepared with 5× Laemmli sample buffer. Samples were run on a gradient gel and transferred for Western blot on 0.45 μm Nitrocellulose membrane. Expression of p21, p27 and the phosphorylated version of STAT1 (pSTAT1) were then evaluated via western blot.

### Results

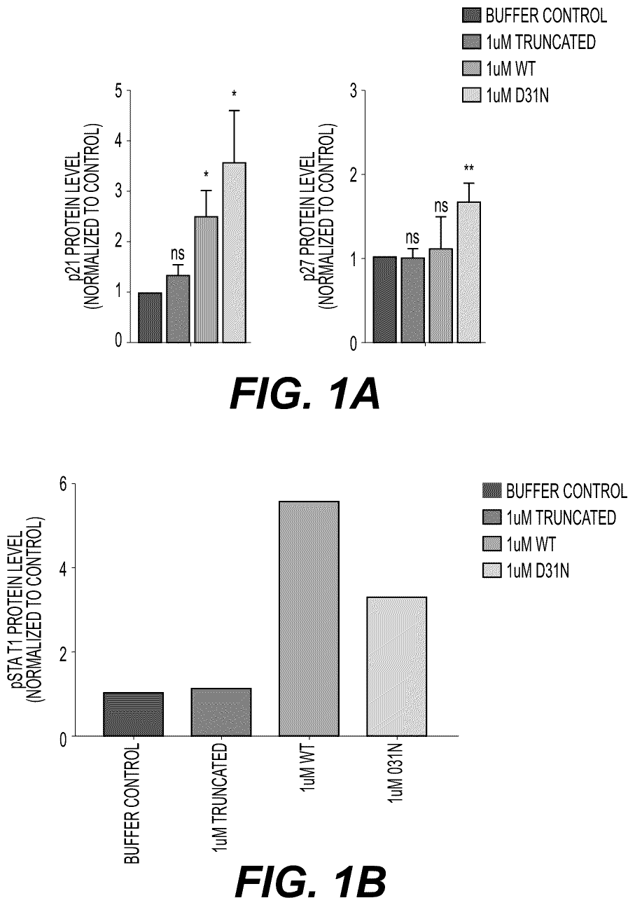

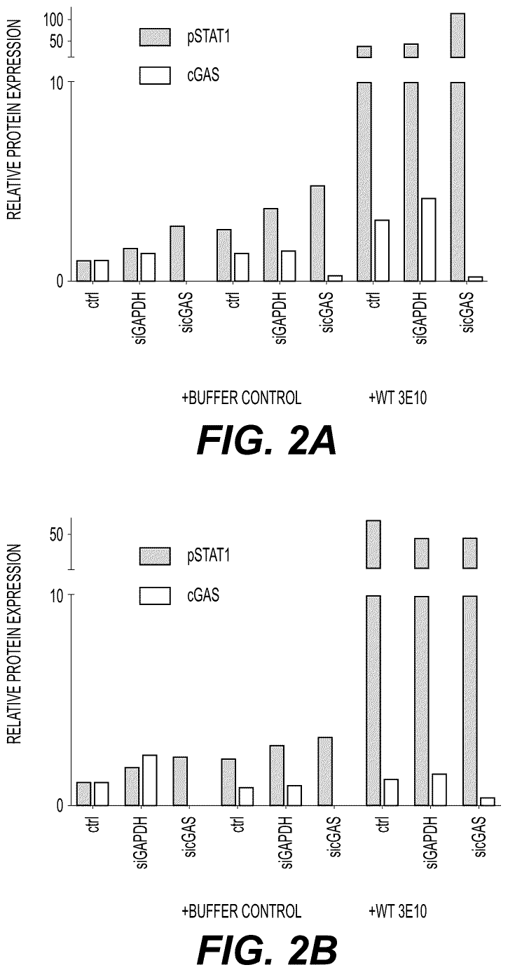

The cell-penetrating antibody 3E10 directly binds to and inactivates RAD51 (Turchick, et al., Nucleic Acids Research, 45 (20): 11782-11799 (2017)). The effect of the 3E10-RAD51 interaction on cellular or replicative senescence was investigated. Results demonstrated that treatment with 3E10 (either the WT or the D31N variant) significantly induced p21 and p27 protein expression in cancer cells (FIG. 1A). Truncated 3E10 did not induce p21 or p27 compared to the buffer treated control (FIG. 1A). These results indicate that functional 3E10 induces senescence in cells with DNA repair deficient backgrounds or cells with excess replication stress, such as cancerous cells.

Absence of normal RAD51 function (due to 3E10 mediated inhibition) is believed to increase accumulation of naked, single-stranded DNA (ssDNA) fragments in the cytoplasm due to replication stress and aberrant DNA repair. To test the hypothesis that the observed 3E10 induced senescence could be associated with activation of innate immunity due to increased cytosolic ssDNA fragments, the status of the cGAS/STING pathway in 3E10 treated cells was examined. The samples from 3E10 treated cells as described above were interrogated for phosphorylated STAT1 (pSTAT1), a well-established marker of cGAS/STING inflammatory pathway activation.

A clear and robust induction of pSTAT1 was observed in 3E10 WT and 3E10 D31N treated samples, but not truncated 3E10, as compared to the buffer control (FIG. 1B). These results indicate that 3E10 stimulates the innate immune response. 3E10's induction of the innate immunity pathway is thus believed to promote anti-tumor immune responses that can be combinable with immune checkpoint therapies, such as anti-PD1, anti-PDL1, and anti-CTLA4 antibodies.

### Example 2: STAT1 Phosphorylation Occurs in a cGAS-Independent Manner

### Materials and Methods

### Cell Culture

B16 murine melanoma and MC38 murine colon carcinoma cells were obtained from ATCC and cultured in DMEM with 10% FBS. MB231 breast cancer cells were cultured in RPMI with L-glutamine and 10% FBS. U251 cells were cultured in DMEM with 10% FBS. MCF10A cGAS knock-out cells were cultured in DMEM: F12 with L-glutamine media supplemented with 5% horse serum, 0.1 μg/ml cholera toxin, 20 ng/ml hEGF, 10 μg/ml insulin, and 0.5 μg/ml hydrocortisone.

RNA Interference siRNAs against gapdh (as a control) or cGAS (ON-TARGETplus SMARTpool reagents, Dharmacon) were transfected into B16 or MC38 or MB231 cells using DharmaFect1 reagent (Dharmacon) following the manufacturer's instructions.

### Immunoblotting

For siRNA experiments cells were transfected with siRNA targeting gapdh or cGAS and then 24 hours later treated with full-length 3E10 at indicated concentrations for 3 days and then pelleted. For experiments using cells lines deficient in cGAS (U251 and MCF10A cGAS-KO) cells were treated with full-length 3E10 for 3 days and then pelleted. Pellets were lysed using AZ lysis buffer (50 mM Tris pH8, 250 mM NaCl, 1% Igepal, 0.1% SDS, 5 mM EDTA, 10 mM Na4P207, 10 mM NaF) supplemented with protease and phosphatase inhibitor cocktails. Protein concentration was determined using the DC protein assay (Bio-Rad) and 50 μg was mixed with sample buffer and boiled for 5 minutes. Samples were loaded and separated using Bio-Rad mini-protean TGX stain-free 4-15% gels. Proteins were transferred by electroblotting onto nitrocellulose. The primary antibodies used were pSTAT1 (Tyr701) (9167, Cell Signaling Technology), cGAS (mouse specific, 31659, Cell Signaling Technology), cGAS (human, 15102, Cell Signaling Technology), actin (8457, Cell Signaling Technology), vinculin (ab130007, Abcam), and gapdh (MAB5718, R&D Systems). Proteins were visualized with horseradish peroxidase-conjugated anti-rabbit immunoglobulin G and a chemiluminescent substrate (Super Signal West Pico Plus, Thermo Scientific). Quantification was performed using ImageJ.

### Results

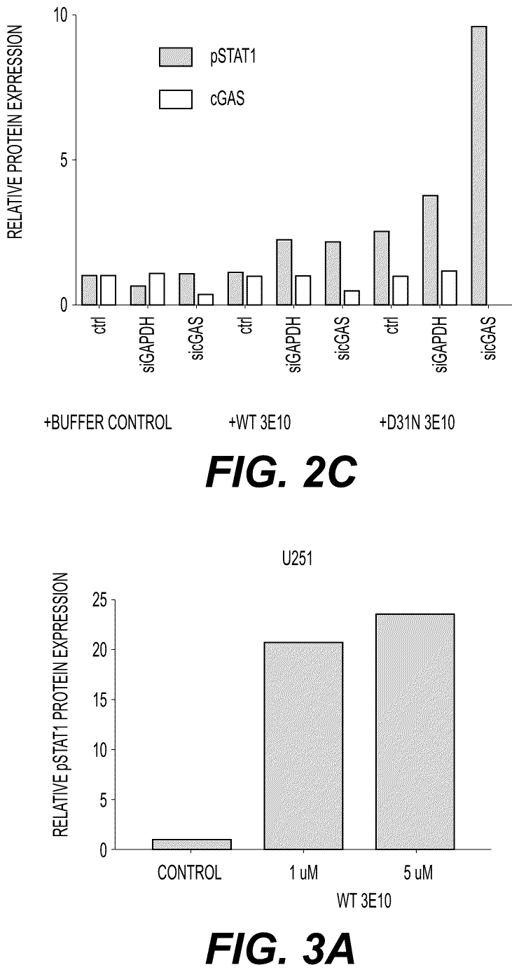

FIG. 2A-2C are quantification of the western blots interrogating STAT1 phosphorylation in cells treated with cGAS targeting siRNA. FIG. 1A shows that siRNA knock-down of cGAS in B16 murine melanoma cells does not prevent STAT1 phosphorylation following treatment with full-length 3E10 (FIG. 2A). Similarly, siRNA knock-down of cGAS in MC38 murine colon carcinoma cells (FIG. 2B), and MB231 breast cancer cells (FIG. 2C) does not prevent STAT1 phosphorylation following treatment with full-length 3E10.

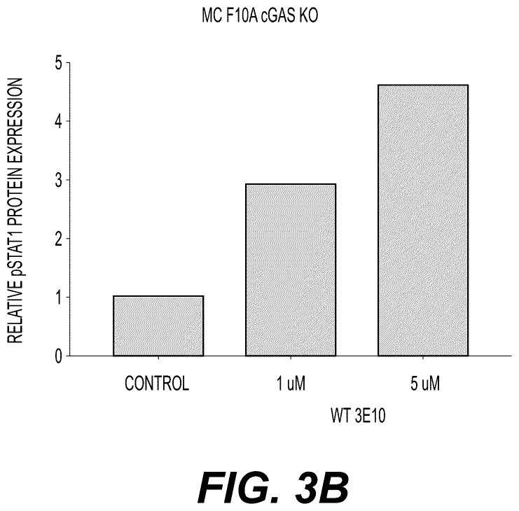

FIGS. 3A-3B are quantification of the western blots interrogating STAT1 phosphorylation in cGAS-deficient cells treated. FIG. 2A shows that cells that are inherently deficient for cGAS (FIG. 3A) or those in which cGAS has been constituently knocked-out (FIG. 3B) still exhibit STAT1 phosphorylation following treatment with full-length 3E10.

Example 1 shows that treatment with full-length 3E10 antibody activates the phosphorylation of STAT1. Example 2 shows that this phosphorylation occurs in a cGAS-independent manner.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A method of treating cancer or an infection comprising administering to a subject in need thereof an effective amount of the combination of
a cell-penetrating binding protein that induces or increase DNA damage or reduces or impairs DNA damage repair, or a combination thereof; and
an immune checkpoint modulator that induces, increases, or enhances an immune response.

2. The method of claim 1 wherein administration of the combination to a subject in need thereof results in a more than additive reduction in one or more symptoms of cancer or infection compared to the reduction achieved by administering the cell-penetrating antibody or the immune checkpoint modulator individually and in the absence of the other.

3. The method of claim 1 wherein the cells associated with the cancer or infection are DNA damage repair deficient.

4. The method of claim 1 wherein the cell-penetrating antibody is administered to the subject 1, 2, 3, 4, 5, 6, 8, 10, 12, 18, or 24 hours, 1, 2, 3, 4, 5, 6, or 7 days, 1, 2, 3, or 4 weeks, or any combination thereof prior to administration of the immune checkpoint modulator to the subject.

5. The method of claim 1 wherein the immune checkpoint modulator is administered to the subject 1, 2, 3, 4, 5, 6, 8, 10, 12, 18, or 24 hours, 1, 2, 3, 4, 5, 6, or 7 days, 1, 2, 3, or 4 weeks, or any combination thereof prior to administration of the cell-penetrating antibody to the subject.

6. The method claim 1 further comprising administering to the subject one or more additional active agents selected from the group consisting of a chemotherapeutic agent, an anti-infective agent, and combinations thereof.

7. The method of claim 1 further comprising surgery or radiation therapy.

8. The method of claim 1 wherein the cell-penetrating binding protein can penetrate the cell, penetrate the nucleus, or a combination thereof without the aid of a conjugate or carrier.

9. The method of claim 1, wherein the cell-penetrating binding protein is an anti-DNA antibody.

10.-11. (canceled)

12. The method of claim 1, wherein the cell-penetrating binding protein inhibits RAD51.

13. The method of claim 1, wherein the cell-penetrating binding protein comprises a 3E10 monoclonal antibody or a cell-penetrating fragment thereof; a monovalent, divalent, or multivalent single chain variable fragment (scFv); or a diabody; or humanized form or variant thereof.

14. The method of claim 13, wherein the cell-penetrating binding protein comprises (i) the CDRs of SEQ ID NO:6 or 7 and SEQ ID NO:1 or 2, or a humanized form thereof; (ii) a heavy chain comprising an amino acid sequence comprising at least 85% sequence identity to SEQ ID NO:6 or 7; and a light chain comprising an amino acid sequence comprising at least 85% sequence identity to SEQ ID NO:1 or 2; (iii) the CDRs of SEQ ID NO:6 or 7 and SEQ ID NO:1 or 2, or a humanized forms thereof; or (iv) a heavy chain comprising an amino acid sequence comprising at least 85% sequence identity to SEQ ID NO:6 or 7; and a light chain comprising an amino acid sequence comprising at least 85% sequence identity to SEQ ID NO:1 or 2.

15. The method of claim 14, wherein the cell-penetrating binding protein comprises the same or different epitope specificity as monoclonal antibody 3E10, produced by ATCC Accession No. PTA 2439 hybridoma.

16. The method of claim 1, wherein the cell-penetrating binding protein comprises one of the following combinations of CDRs:
SEQ ID NO:30, SEQ ID NO:31, SEQ ID NO:33 and SEQ ID NO:34, SEQ ID NO: 36, SEQ ID NO:37;
SEQ ID NO:30, SEQ ID NO:32, SEQ ID NO:33 and SEQ ID NO:34, SEQ ID NO: 36, SEQ ID NO:37;
SEQ ID NO:30, SEQ ID NO:32, SEQ ID NO:33 and SEQ ID NO:34, SEQ ID NO: 36, SEQ ID NO:37; or,
SEQ ID NO:30, SEQ ID NO:32, SEQ ID NO:33 and SEQ ID NO:35, SEQ ID NO: 36, SEQ ID NO:37.

17. The method of claim 15, comprising a recombinant antibody having the paratope of monoclonal antibody 3E10.

18-20. (canceled)

21. The method of claim 1, wherein the immune checkpoint modulator induces an immune response against the cancer or infection.

22-23. (canceled)

24. The method of claim 21, wherein the immune checkpoint modulator is selected from the group consisting of PD-1 antagonists, PD-1 ligand antagonists, and CTLA4 antagonists.

25. (canceled)

26. The method of claim 1, wherein the immune checkpoint modulator is an antibody.

27-29. (canceled)

30. A method of treating cancer comprising administering to a subject in need thereof an effective amount of the combination of
a cell-penetrating anti-DNA binding protein which comprises:
a V_{H} comprising an amino acid sequence as shown in any one of SEQ ID NOs: 9, 11, or 45 to 52 and a V_{L} comprising an amino acid sequence as shown in any one of SEQ ID NOs: 3 or 5 or 53 to 58; or,
an amino acid sequence as shown in any one of SEQ ID NOs: 61-76; and,
an immune checkpoint modulator which is an anti-PD1 an anti-PDL1, or an anti-CTLA4 antibody.

31-33. (canceled)

34. A pharmaceutical composition comprising an effective amount of the combination of a cell-penetrating antibody that induces or increase DNA damage or reduces or impairs DNA damage repair, or a combination thereof; and
an immune checkpoint modulator that induces, increases, or enhances an immune response,
wherein administration of the pharmaceutical composition to a subject in need thereof reduces one or more symptoms of cancer or an infection to a greater degree than administering to the subject the same amount of cell-penetrating antibody alone or the same amount of immune checkpoint modulator alone.

35-53. (canceled)
